(19) United States
(12) Patent Application Publication
(10) Pub. No.: US 2022/0119828 A1
(43) Pub. Date: Apr. 21, 2022

(54) NUCLEIC ACID FOR EDITING GENOME OF PLANT CELL AND USE THEREOF

(71) Applicant: Bio Palette Co., Ltd., Kobe-shi, Hyogo (JP)

(72) Inventors: Zenpei SHIMATANI, Kobe (JP); Rie TERADA, Nagoya (JP)

(73) Assignee: Bio Palette Co., Ltd., Kobe-shi, Hyogo (JP)

(21) Appl. No.: 17/432,817

(22) PCT Filed: Feb. 21, 2020
(86) PCT No.: PCT/JP2020/006977

§ 371 Date: Aug. 20, 2021

(30) Foreign Application Priority Data
Feb. 22, 2019 (JP) 2019-030971

(51) Int. Cl. C12N 15/82

(52) U.S. Cl. CPC C12N 15/8213

(57) ABSTRACT

The present invention provides a nucleic acid comprising: (1) a transposon; and (2) a sequence homologous to at least a part of a target region of a double-stranded DNA of a plant cell, wherein the transposon comprises a marker gene and a gene encoding a transposase operably linked to an inducible promoter.

### TECHNICAL FIELD

The present invention relates to a nucleic acid for editing the genome of a plant cell comprising a transposon and a method for producing a plant cell having a mutation in a target region of a double-stranded DNA by using the nucleic acid.

### BACKGROUND ART

There is a need for quick and highly versatile new plant breeding techniques in order to promote increased food production and improvement of the environment in a sudden change in the agriculture situation such as rapid growth of population or climate changes in recent years. A typical example of the techniques is molecular breeding utilizing random gene recombination. However, with the conventional plant gene recombination techniques, marker genes such as antibiotic-resistant genes need to be incorporated into a plant genome together with a gene of interest, wherein there is a problem of inability to control the region in the plant genome in which those foreign genes are incorporated and the number of copies of the foreign genes that are incorporated. In order to solve this problem, genome editing techniques utilizing artificial nucleases such as TALEN or CRISPR/Cas are attracting attention (e.g., Non Patent Literature 1). With these genome editing techniques, a double strand break (DSB) is mainly caused in a target DNA in a plant genome to induce a non homologous end joining (NHEJ) repairing mechanism in the plant cell. During the process, a mutation randomly occurs, which results in loss of the function of the target gene. In order to modify a target gene as designed, a template DNA is supplied to a region where a DSB has occurred due to an artificial nuclease to be incorporated into the plant genome side using homologous recombination (HR). However, there is still a high technical barrier because higher plants have an extremely low frequency of HR relative to NHEJ (Non Patent Literature 2), and both expression of an artificial nuclease and supply of a template DNA need to be achieved in the same cell. Further, when artificial nucleases are used, the off-target effect that causes a DSB in a place other than a target gene and the possibility of a large-scale structural mutation resulting therefrom cannot be avoided. Moreover, it is very difficult to introduce an artificial nuclease or the like to higher plant cells with an RNA and a protein. Thus, in order to utilize the genome editing techniques, it is necessary to incorporate a necessary gene group into a plant genome as a DNA in advance and express the gene group.

In order to develop a modified plant body obtained by the conventional gene recombination or genome editing techniques as a new variety, an artificial DNA sequence contained in the genome needs to be removed. One of the major measures is a technique utilizing gene segregation. However, this technique requires artificial crossing or the like and requires plant raising for a few years. Further, this technique cannot be applied to vegetative-propagation plants. Meanwhile, bacteriophage P1-derived Cre-loxP- or yeast-derived FLP-FRP-systems and the like are known as a method for deleting an artificial DNA sequence by utilizing a site-specific recombination technique (e.g., Non Patent Literature 3). However, these systems have a problem that a foreign DNA sequence having several tens of bp remains in the plant genome as a trace.

### CITATION LIST

### Non Patent Literature

[NPL 1] Jung C. et al., Plant Breeding, 137:1-9 (2018)
[NPL 2] Paszkowski J. et al., EMBO J., 7(13):4021-4026 (1988)
[NPL 3] Dang T. T. et al., Plant Cell Physiol, 54(12):2058-2070 (2013)

### SUMMARY OF INVENTION

### Technical Problem

Thus, the problem to be solved by the present invention is to provide a minute and quick plant breeding technique that is able to introduce a mutation into a plant genome without leaving a trace of an exogenous artificial DNA sequence.

### Solution to Problem

The present inventors conceived of an idea that it may be possible to improve the frequency of successful target gene modification and further reduce necessary man-hours at the same time by improving a gene targeting method based on a positive-negative selection approach using Cre-loxP, and incorporating an autonomous marker deleting mechanism using a modified transposon instead of Cre-loxP. The present inventors advanced a research based on the idea to find that it is possible to autonomously perform two-stage target gene modification with one gene recombinant manipulation by using an Ac/Ds system, wherein an exogenous gene can be removed from the modified genome without leaving a trace of the exogenous gene, and that this method can create a new plant line having a useful phenotype in only about 10 months. The present inventors conducted further researches based on these findings to complete the present invention.

Specifically, the present invention is as follows.

[1] A nucleic acid comprising:

(1) a transposon; and

(2) a sequence homologous to at least a part of a target region of a double-stranded DNA of a plant cell,

wherein the transposon comprises a marker gene and a gene encoding a transposase operably linked to an inducible promoter.

[2] The nucleic acid of [1], wherein the transposon is derived from a transposon selected from the group consisting of a Ds element, an Ac element, an Spm-s element, an En1 element, an Spm-I8 (dSpm) element, an Mu element, a Tam1 element, a Tam2 element, a Tam3 element, an nDart element, a Dart element, and a PiggyBac element.
[3] The nucleic acid of [1] or [2], wherein the transposon is derived from a Ds element or an Ac element, and the transposase is AcTPase or a variant thereof.
[4] The nucleic acid of any of [1] to [3], wherein the marker gene includes a gene encoding a fluorescent protein.
[5] The nucleic acid of any of [1] to [4], wherein the inducible promoter is a heat-shock inducible promoter.
[6] A method for producing plant cells that have a mutation in a target region of a double-stranded DNA and do not have any of an exogenous transposon and a gene encoding a transposase, the method comprising:

(1) the step of selecting plant cells introduced with the nucleic acid of any of [1] to [5];

(2) the step of culturing the plant cells selected in step (1) under a condition wherein the inducible promoter is activated; and

(3) the step of selecting a cell in which expression of the marker protein is eliminated from a population comprising the plant cells cultured in step (2).

[7] The method of [6], wherein the plant cells are cells of a monocotyledon.
[8] The method of [7], wherein the monocotyledon is a gramineous plant.
[9]

A plant cell obtained by the method of any of [6] to [8]or a plant body comprising the cell.

### Advantageous Effects of Invention

With the plant genome editing technique of the present invention, two-stage target gene modification can be autonomously materialized with one gene recombinant manipulation. Specifically, a wide range of a DNA sequence is modified as designed by gene targeting via homologous recombination in primary modification, and an artificial DNA sequence such as a transposon or a gene encoding a transposase is autonomously removed by a transposon system in secondary modification. A plant made in this manner in which the artificial DNA sequence is removed also exerts an advantageous effect that it can be excluded from the subject of the Cartagena Protocol. Since an exogenous transposon, a transposase and the like can be removed through the above-described secondary modification, it is not necessary to make a multiple transformant or perform backcross with a wild type to remove the above-described exogenous factors as in the conventional techniques. Thus, the present genome editing technique can minutely and quickly modify only a target gene, so that said technique is extremely useful as a practical plant breeding technique.

### BRIEF DESCRIPTION OF DRAWINGS

FIG. 1 shows the T-DNA structure of the binary vectors used for evaluation of a heat-shock inducible Ac/Ds-mediated marker excision system. (A) A schematic diagram of a pZEN30PC construct, wherein p35S shows a cauliflower mosaic virus (CaMV) 35S promoter (the sequence set forth in SEQ ID No: 37 or 38), GUSPlus shows a β-glucuronidase gene, iCAT shows intron 1 of a castor bean Catalase, tNOS shows a nopaline synthase terminator, pAct1 and iAct1 show a rice Actin1 promoter (the sequence set forth in SEQ ID NO: 43) having an intron of rice Actin1, hpt shows a hygromycin phosphotransferase gene, and t35S shows a CaMV 35S terminator (the sequence set forth in SEQ ID NO: 40 or 41). (B) A schematic diagram of a pZEN30NC construct, wherein ΔEn shows a functional transcription stop sequence derived from a maize En/Spm transposon, and Ds_5 and Ds_3 show a 300 bp terminal region of a maize Ac element. (C) A schematic diagram of a pZEN31E construct, wherein pHSP shows a promoter region of Oshsp16.9C, which is a heat-shock protein of rice, NLS shows a nuclear localization signal of an SV40 large T antigen, AcTPase4xOs-int shows a codon-optimized ORF of enhanced AcTPase having an intron, and RB and LB show a border sequence on the right side and a border sequence on the left side, respectively.

FIG. 2 shows a method for evaluating the heat-shock inducible Ac/Ds-mediated marker excision system in a callus of rice and the results thereof. (A) A schema of a GUS reporter assay using pZEN31E. ΔEn was inserted as a transcription terminator to functionally disrupt GUSPlus. Ds_5 and Ds_3 show a 300 bp terminal region of a maize Ac element. (B) The frequency and behaviors of heat-shock inducible Ac/Ds-mediated excision of a removal unit can be visualized by the GUS-reporter assay. Excision can be confirmed by PCR analysis using primers (Table 5) shown as horizontal arrows. (C) The PCR analysis results of a callus of transformed rice. Fragments amplified by PCR represent the removal unit being excised from the T-DNA region of pZEN30NC and 31E in a heat-shock inducible manner. (D) The results of the GUS-reporter assay of a genetically recombinant rice callus using histochemical staining with X-Gluc.

FIG. 3 shows the design and construct of a gene targeting vector having an autonomous marker excision system in a rice callus. (A) A model of a gene to be modified. (B) Cloning of a homology region. Ds_5 (the sequence set forth in SEQ ID NO: 34) and Ds_3 (the sequence set forth in SEQ ID NO: 35) are each a terminal region of an Ac element which is required for marker excision by an AcTpase4xOs transposase. (C) A homology region having a removal unit which comprises a positive marker gene (Hm^{r}). (D) Integration of heat-shock inducible AcTPase4xOs and constitutively active EGFP by a gateway LR reaction by a gateway LR reaction. (E) A figure of a complete gene targeting vector having an autonomous marker excision system. The constructed components are transferred between two diphtheria toxin A subunit genes (DT-A) (the sequence set forth in SEQ ID NO: 39) under the control of a constitutive promoter.

FIG. 4 shows primary modification of OsClpP5 by gene targeting mediated by homologous recombination. (A) An OsClpP5 gene in a rice genome. White boxes and black boxes show an untranslated region (UTR) and an exon, respectively. (B) The structure of pOsClpP5KO-AcDs, which is a gene targeting vector. The left and right bars represent a DNA sequence corresponding to a homology region carried by the vector. The star in the 5′-homology region shows the position of a new PvuII site and a desired mutation that results in disruption of a splice donor site of intron 1 of OsClpP5. (C) The structure of OsClpP5 modified by gene targeting. The horizontal lines marked with Clp 5J and Clp 3J show 5′ and 3′ junction fragments made by homologous recombination. The adjacent black arrows show primers that were used for PCR screening of callus lines of a target (Table 1). (D) The PCR screening results of gene-targeted (GT) callus lines. P shows a control plasmid comprising Clp 5J or Clp3J (FIG. 4C), N shows a callus of non-transformed rice variety “Nipponbare”, and lanes 1 to 8 show a GT callus transformed with pOsClpP5KO-AcDs. (E) EGFP expression in the GT callus. Scale bar: 2.5 mm.

FIG. 5 shows excision of a positive marker from OsClpP5 modified by a heat-shock inducible Ac/Ds system. (A) The state of heterozygosis of an OsClpP5 gene of a target in the GT callus. The black arrows show primers that were used for PCR analysis to detect the presence of a removal unit (Table 1). (B) The modified OsClpP5 gene after excision of the removal unit mediated by Ac/Ds. The black arrows show primers that were used for CAPS and DNA sequencing analysis (Table 1 and FIG. 6A). (C) Proliferation of GT callus lines over time after heat-shock treatment. The proliferative cell lines without an EGFP signal were surrounded with a dotted line. Scale bar: 2.5 mm. (D) The presence of a removal unit in the GT callus lines. The arrow shows a DNA fragment amplified by PCR using a primer pair (En No. 2-F/pHSP J-R) (Table 1 and FIG. 5A). Proliferative calli were used for analysis regardless of the presence or absence of an EGFP signal.

FIG. 6 shows transfer of the modified OsClpP5 gene to T₀ plants. (A) CAPS analysis of a target region in T₀ plants. A primer pair (Clp Ex F-6/Clp Ex-R) (Table 1 and FIG. 5B) amplifies a characteristic PCR fragment comprising a region (0.92 kb) of OsClpP5 intron 1. A PCR amplification product derived from the modified OsClpP5 KO gene was digested into two fragments (0.56 kb and 0.36 kb) by PvuII. WT shows a wild-type “Nipponbare” plant. (B) The spectra of Sanger sequencing of the OsClpP5 locus. The arrows show an overlapping heterogenic mutation. (C) EGFP expression in root tissue of T₀ plants. Regenerates derived from the wild type and the GT callus lines that were treated or not treated with heat shock are shown. Scale bar: 1.25 mm. (D) The presence of a removal unit in T₀ plants. P shows pOsClpP5KO-Con5, which is a control plasmid, while N shows a callus of non-transformed “Nipponbare”.

FIG. 7 shows the growth situation of T₀OsClpP5 KO plants. (A) T₀ plants in the ear emergence period. Left: Wild-type “Nipponbare”. Middle: OsClpP5 KO T₀ plants derived from the callus lines that were not treated with heat shock. Right: OsClpP5 KO T₀ plants derived from the callus lines that were treated with heat shock. Scale bar: 12.5 cm. (B) The relative effect of the heat-shock treatment for the plant height of T₀ plants. The height of the wild-type plants which germinated from seeds and T₀ plants regenerated from marker-free callus lines were measured in their respective ear emergence period. The numbers on the horizontal axis show the length of time of the heat-shock treatment. 0 minute shows no heat-shock treatment, while 40 minutes, 60 minutes, and 90 minutes show the time during which the callus lines were maintained at 42° C. for Ac/Ds-mediated marker excision. Each dot shows individual plants. (C) The relative effect of the heat-shock treatment for seed fertility of T₀ plants. The fertility of each plant was evaluated by averaging the fertility of two ears.

FIG. 8 shows heredity and segregation of the modified OsClpP5 gene in the T₁ generation. (A) Segregation of a phenotype in T₁ plants derived from T₀ plants having a heterozygosis-type OsClpP5 KO mutation. (B) A segregated albino plant having a homozygosis-type osclpp5 mutation. Scale bar: 5 mm. (C) The presence of a removal unit in the segregated albino plant. P shows pOsClpP5KO-Con5, which is a control plasmid, while WT shows a wild-type “Nipponbare” plant. (D) The results of CAPS analysis of a target region in the segregated plants. Lanes 1 to 3 show a segregated wild-type plant, lanes 4 to 6 show a heterozygosis-type plant, and lanes 7 to 9 show a homozygosis-type osclpp5 plant that exhibits an albino phonotype. The filled arrow indicates a PCR amplification product that does not have a PvuII site. The white arrows indicate a fragment amplified from an albino plant and digested with PvuII. (E) The spectra of Sanger sequencing of the OsClpP5 locus. The arrows show an introduced mutation.

FIG. 8 shows heredity and segregation of the modified OsClpP5 gene in the T₁ generation. (F) The footprint sequence of Ac/Ds-mediated marker excision that was detected among osclpp5 disruption strains that exhibit an albino phenotype.

FIG. 9 shows primary modification of OsRacGEF1 by gene targeting which is mediated by homologous recombination. (A) An OsRacGEF1 gene in a rice genome. (B) The structure of pGEF1S549D-AcDs, which is a gene targeting vector. White boxes and black boxes show an untranslated region (UTR) and an exon, respectively. The left and right bars represent a DNA sequence corresponding to a homology region carried by the vector. The star in the 5′-homology region shows the position of a desired mutation that results in an S549D amino acid substitution of an OsRacGEF1 protein. The arrow indicates a new BssHII site adjacent to the S549D mutation. (C) The structure of OsRacGEF1 modified by gene targeting. The horizontal lines marked with GEF 5J and GEF 3J show 5′ and 3′ junction fragments made by homologous recombination. The adjacent black arrows show primers that were used for PCR screening of callus lines of a target (Table 2). (D) The PCR screening results of GT callus lines. P shows a control plasmid comprising GEF 5J or GEF 3J (FIG. 9C), N shows a callus of non-transformed “Kinmaze”, and lanes 1 to 8 show a GT callus transformed with pGEF1S549D-AcDs.

FIG. 10 shows excision of a positive marker from modified OsRacGEF1 by a heat-shock inducible Ac/Ds system. The black arrows show primers that were used for PCR analysis to detect the presence of a removal unit (Table 2). (A) The modified OsRacGEF1 gene before Ac/Ds-mediated removal unit excision. (B) The modified OsRacGEF1 gene after Ac/Ds-mediated removal unit excision. The black arrows show primers that were used for CAPS and DNA sequencing analysis (Table 2). (C) Proliferation of GT callus lines over time after heat-shock treatment. The proliferative cell lines without an EGFP signal were surrounded with a dotted line. Scale bar: 2.5 mm. (D) PCR and CAPS analysis of OsRacGEF1 S549D GT T₀ plants. The presence of a removal unit was analyzed by PCR using a primer pair (En No. 2-F/pHSP J-R) (Table 2 and FIG. 10A). A DNA fragment comprising a desired mutation was amplified by PCR using a primer pair (GEF Ex F-5/GEF Ex-R) and digested with BssHII. The filled arrows indicate a PCR amplification product from unmodified OsRacGEF1. The white arrows indicate a fragment amplified from a modified target and digested with BssHII. P shows pGEF1S549D-Con, which is a control plasmid, while N shows a callus of non-transformed “Kinmaze”. (E) The spectra of Sanger sequencing of the OsRacGEF1 locus. The arrows show an introduced mutation.

FIG. 11 shows the results of cloning sequence analysis of a target region of an OsRacGEF1 S549D variant callus.

FIG. 12 shows a flow chart of making T₁ plants from calli introduced with the nucleic acid of the present invention.

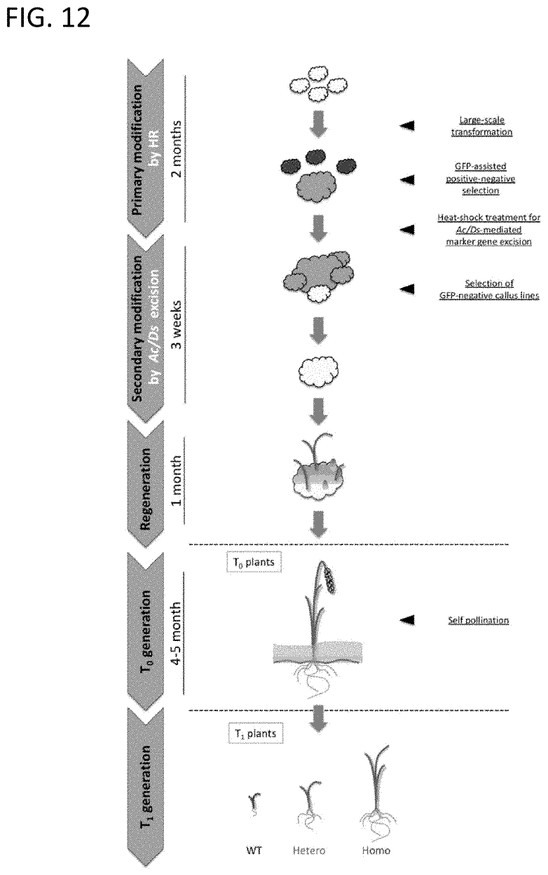

FIG. 13 shows a flow chart of gene modification that occurs while T₁ plants are made from calli introduced with the nucleic acid of the present invention.

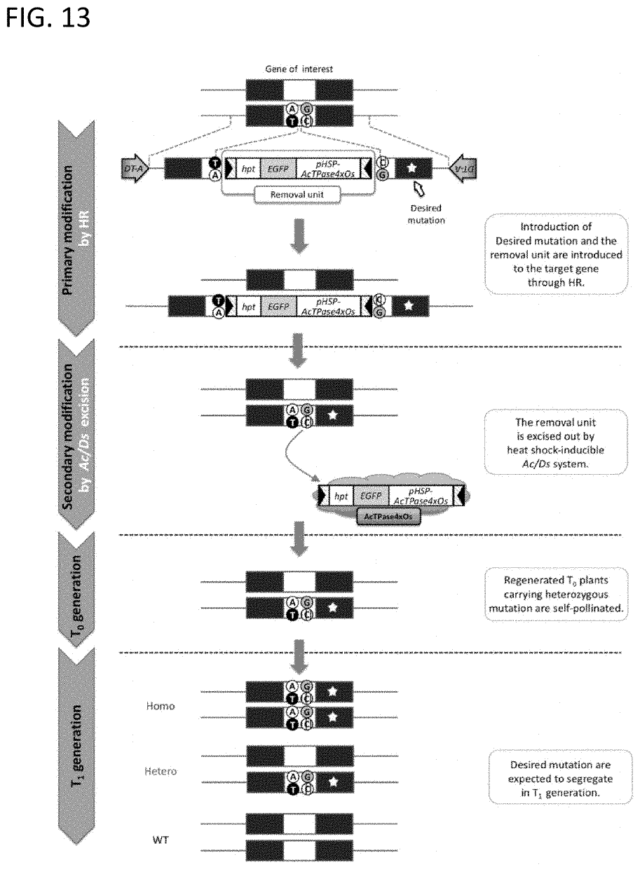

FIG. 14 shows the results of RNA expression analysis in a clpP5-Δspl secondary modification callus-derived T1 rice budding leaf. A: A schema of a genotype and an mRNA in the T1 generation of an OsClpP5 modification line. B: A part of the mRNA sequence of an OsClpP5 variant. TGA: shows a stop codon, and small letter: shows a first intron. C: An electrophoresis image of an OsClpP5 cDNA. WT: shows wild type, HT: shows hetero, and HM: shows homo.

FIG. 15 shows the cDNA sequence of an OsClpP5 variant T₁ plant body. A: A splicing schema of an OsClpP5 gene in a wild-type plant body segregated in the T1 generation. B: The base sequence of the OsClpP5 gene and a cDNA of a wild-type plant body segregated in the T1 generation. C: A splicing schema in the osclpP5 homozygosis type. D: The base sequence of a gene and a cDNA in the osclpP5 homozygosis type.

### DESCRIPTION OF EMBODIMENTS

### 1. Nucleic Acid for Genome Modification of a Plant Cell

The present invention provides a nucleic acid for genome modification of a plant cell, comprising a transposon (which may be hereinafter referred to as the “nucleic acid of the present invention”). The transposon comprises a marker gene and a gene (nucleotide sequence) encoding a transposase (a transposon comprising these sequences may be referred to as the “transposon of the present invention”). Further, the nucleic acid of the present invention comprises two types of regions that are homologous to at least a part of a target region in a plant cell for causing homologous recombination with the target region (specifically, the regions on the upstream side and the downstream side in the target region) (these regions may be hereinafter called “homology regions”, and when each homology region is distinguished, they may be referred to as the “5′ homology region” and/or “3′ homology region”). A region consisting of the transposon and the homology regions may be hereinafter referred to as the “recombinant sequence”. The definition of the plant cell, the type of the plant and the like are as described in 2. below.

As used herein, a “transposon” means a DNA sequence that is removed by a transposase. A terminal inverted repeat (TIR) which is recognized by a transposase is placed on both ends of the transposon. Examples of the transposon system used for the present invention include a DNA type transposon system although the transposon system used for the present invention is not particularly limited as long as it can remove the transposon introduced to a plant cell without leaving a trace in the genome of the plant cell. Examples of the DNA type transposon system include an Ac/Ds system derived from maize, Arabidopsis thaliana, tobacco, tomato or petunia, an EnSpm/CACTA system derived from maize, Arabidopsis thaliana or soy bean, an Mu system derived from maize, a Tam1/Tam2 system derived from snapdragon, a Tam3 system, a Dart/nDart system derived from rice, a PiggyBac system derived from Trichoplusia ni and the like. Thus, examples of the transposon used for the present invention include a sequence in which a marker gene and a gene encoding a transposase are inserted to a natural-type transposon such as a Ds (Dissociation) element (e.g., Ds1, Ds2, Ds6), an Ac (Activator) element of an Ac/Ds system; an Spm-s element, an En1 element, an Spm-I8 (dSpm) element or the like of an EnSpm/CACTA system; an Mu element (e.g., Mu1, Mu1.7, Mu7 or the like) of an Mu system; a Tam1 element, a Tam2 element or the like of a Tam-1/Tam-2 system; a Tam3 element of a Tam3 system or the like; a Dart element, nDart element of a Dart/nDart system; a PiggyBac element, or a transposon in which at least a part of the sequence of the natural-type transposon is modified (also referred to as an artificial-type transposon), and/or a sequence in which a part of the transposon is substituted with the sequence of these genes. Examples of the above-described artificial-type transposon include a combination of the above-described elements (e.g., a sequence in which a TIR and a subterminal region of a Ds element are substituted with a TIR and a subterminal region of an Ac element) and the like. As used herein, a transposon resulting from subjecting a natural-type or an artificial-type transposon to modification such as insertion or substitution of a sequence as described above, or any sequence comprising a TIR recognized by each transposase is referred to as transposon derived from each natural-type transposon. For example, a transposon resulting from subjecting a natural-type or an artificial-type Ds element to modification is referred to as transposon derived from a Ds element. Further, the transposase used for the present invention includes AcTPase (Genebank Accession No: X05424.1) that can remove a Ds element and Ac element, PBase (Genebank Accession No: EF587698.1) that can remove a PiggyBac element, a transposase encoded by a part of each natural-type transposon described above, and the like although the transposase used for the present invention is not particularly limited as long as it can remove the above-described transposon.

Further, a transposase may be a wild type, and may be a variant thereof having an activity to remove a transposon. The variant of a transposase includes a protein having a transposon removal activity and consisting of an amino acid sequence resulting from substitution, deletion, addition, and/or insertion of one or a few (e.g., two, three, four, five, six, seven, eight, nine, ten) amino acids in the amino acid sequence of a wild-type transposase, or a protein having a transposon removal activity and consisting of an amino acid sequence having 90% or greater (e.g., 91%, 92%, 93%, 94%, 95%, 96%, 97%, 98%, 99% or greater) identity to the above-described amino acid sequence. Examples of the variant of a transposase include AcTPase4x (Lazarow K. et al., Genetics, 191(3):747-756 (2012)) with an improved transposon removal activity due to substitution of four amino acid residues (specifically, E249A/E336A/D459A/D545A), which is a variant of AcTPase.

The amino acid sequence identity can be calculated using NCBI BLAST (National Center for Biotechnology Information Basic Local Alignment Search Tool) (https://blast.ncbi.nlm.nih.gov/Blast.cgi) of an identity calculation algorithm under the following condition (expectancy=10; gap allowed; matrix=BLOSUM62; filtering=OFF). A sequence of the full length of a transposase is compared to another sequence in order to determine the identity.

The present inventors found that when AcTPase is used as a transposase, it is possible to remove a transposon without leaving a trace by replacing a base of one chain in contact with both ends of the region of the transposon with a base complementary thereto (e.g., if a base pair in contact with one end is A:T, replace it with T:A, and if a base pair in contact with one end is G:C, replace it with C:G). Thus, when AcTPase is used as a transposase, it is generally necessary to replace a base of one chain in contact with both ends of the region of a transposon with a base complementary thereto in order to remove the transposon without leaving a trace in the genome of a plant cell. Further, a gene encoding the transposase is desirably added with a sequence encoding a nuclear localization signal (NLS) (e.g., SV40-derived NLS encoding sequence (the sequence set forth in SEQ ID NO: 33)) that can function in the plant cell.

As used herein, a transposon not leaving a trace in the genome of a plant cell shall encompass not only the case where no trace of the transposon (e.g., the transposon, a sequence encoding a transposase, a footprint caused by removal of the transposon, or the like) is found in the genome but also the case where deletion, substitution, and/or insertion of 10 or less (e.g., 9, 8, 7, 6, 5, 4, 3, 2, or 1) nucleotide residues is found.

A TIR positioned on both ends of the transposon of the present invention can be appropriately selected depending on the type of the transposase. Specifically, when AcTPase is used, examples of the sequence of a TIR on one terminal include CAGGGATGAAA (SEQ ID NO: 1), TAGGGATGAAA (SEQ ID NO: 2), GAGGGATGAAA (SEQ ID NO: 3), TAGAGATGAAA (SEQ ID NO: 4), GAGCTATGAAA (SEQ ID NO: 5) and the like while the sequence of a TIR on the other terminal includes TTTCATCCCTA (SEQ ID NO: 6), TTTCATCTGAG (SEQ ID NO: 7), TTTCATCCCTA (SEQ ID NO: 8), TTTCATCCCTG (SEQ ID NO: 9), TTTCATCTCTA (SEQ ID NO: 10) and the like, wherein these sequences can be appropriately combined to be used. Examples of the above-described combination include the sequence set forth in SEQ ID NO: 1/the sequence set forth in SEQ ID NO: 6, the sequence set forth in SEQ ID NO: 1/the sequence set forth in SEQ ID NO: 7, the sequence set forth in SEQ ID NO: 2/the sequence set forth in SEQ ID NO: 8, the sequence set forth in SEQ ID NO: 2/the sequence set forth in SEQ ID NO: 9, the sequence set forth in SEQ ID NO: 2/the sequence set forth in SEQ ID NO: 10, the sequence set forth in SEQ ID NO: 3/the sequence set forth in SEQ ID NO: 9, the sequence set forth in SEQ ID NO: 4/the sequence set forth in SEQ ID NO: 8, the sequence set forth in SEQ ID NO: 5/the sequence set forth in SEQ ID NO: 8 and the like. When an EnSpm/CACTA system is used, examples of the sequence of a TIR on one terminal include CACTACAAGAAAA (SEQ ID NO: 11), CACTACAACAAAA (SEQ ID NO: 12), CACTACAAAAAAA (SEQ ID NO: 13), CACTATAAGAAAA (SEQ ID NO: 14), CACTACGCCAAAA (SEQ ID NO: 15), CACTACCGGAATT (SEQ ID NO: 16) and the like while the sequence of a TIR on the other terminal includes the complementary sequences (SEQ ID NO: 17 to 22) of each of the above-mentioned sequences. When a PiggyBac system is used, examples of the sequence of a TIR on one terminal include CCCTAGAAAGATA (SEQ ID NO: 23), CCCTAGAAAGATAGTCTGCGTAAAATTGACGCATG (SEQ ID NO: 24), CATGCGTCAATTTTACGCAGACTATCTTTCTAGGG (SEQ ID NO: 25) and the like while the sequence of a TIR on the other terminal includes the complementary sequences (SEQ ID NO: 26 to 28) of each of the above-mentioned sequences. The above-described specific sequences are exemplification, and a sequence exhibiting a high identity (e.g., 90% or greater) to the above-described sequences also can be used as a TIR as long as it is recognized by a transposase. Further, those skilled in the art can appropriately select a sequence even when a transposon system other than those mentioned above is used.

Further, one end or both ends of the transposon of the present invention may comprise a subterminal region (STR) adjacent to the above-described TIR which is joined to a transposase apart from the TIR. A STR generally consists of 100 to 300 bp. When AcTPase is used as a transposase, the STR preferably comprises, for example, at least one sequence (preferably three or more sequences) consisting of AACGG necessary for joining of AcTPase. In a preferred embodiment, a transposon comprising an IVR and a STR includes a transposon having the sequence set forth in SEQ ID NO: 34 (as used herein, it is referred to as “Ds_5” for convenience sake) on one terminal and having the sequence set forth in SEQ ID NO: 35 (as used herein, it is referred to as “Ds_3” for convenience sake) on the other terminal, or a transposon having a sequence having a high identity (e.g., 90% or greater, 95% or greater, 96%, 97%, 98%, 99% or greater) to these sequences, and the like. Those skilled in the art can appropriately select the sequence of the STR depending on the type of the transposase.

The number of nucleotides of the transposon of the present invention may be any number of nucleotides that can be excised out by a transposase. Although the number of nucleotides of the transposon of the present invention depends on the number of nucleotides of a marker gene, a transposase or the like described below, the number is generally 1500 to 10000 nucleotides and is preferably 3000 to 8000 nucleotides.

As used herein, a “target region” means a region on the genome of a plant cell in which homologous recombination with a recombinant sequence occurs. A desired mutation is introduced to at least one of the 5′ homology region and the 3′ homology region of a recombinant sequence in advance, and homologous recombination is caused between a sequence having the mutation and a target region, whereby the desired mutation can be introduced to the target region of a plant cell. A target region can be optionally selected from a region on the genome of a plant cell, may be a region encoding a protein, or may be a DNA encoding a noncoding RNA such as a functional RNA. Alternatively, a target region may be not only a region that does not encode a protein or a noncoding RNA (such as an UTR) but also a region that adjusts the expression and the chromosome structure of a transcriptional product encoding a protein or a noncoding RNA. Further, a target region is generally endogenous, but may be a DNA inserted to a genome DNA of a plant cell in a foreign manner.

The sequence of a homology region may be a sequence that is completely identical to the region of at least a part of a target region, but may be a sequence preferably having 80% or greater (e.g., 85% or greater, 90% or greater, 95% or greater, 96% or greater, 97% or greater, 98a or greater, 99% or greater) identity to the completely identical sequence as long as homologous recombination can occur in a plant cell.

Further, the number of nucleotides of a homology region may be any number that can cause homologous recombination between the homology region and a target region. Each of the both sides of a transposon is generally added with a homology region consisting of 500 to 7000 nucleotides (preferably 1000 to 5000 nucleotides, more preferably 1500 to 4000 nucleotides, and further preferably about 3000 nucleotides (e.g., 2500 to 3500 nucleotides)).

A “mutation” which is introduced to a target region is not particularly limited, and may be a null mutation such as a nonsense mutation, a frameshift mutation, an insertion mutation or a splice site mutation, or may be a silent mutation. Further, examples of a mutation in a target region include deletion, substitution, addition, and/or insertion of one or more (e.g., 2, 3, 4, 5, 6, 7, 8, 9, 10 or more) nucleotides in the region. Further, a mutation may be introduced to only one site or may be introduced to multiple sites (e.g., 2, 3, 4, 5 or more sites). When a mutation is introduced to multiple sites, the mutation introduction sites may be close to each other or may be about hundreds or thousands of bases away from each other.

A “marker gene” comprised in a transposon may be any gene whose expression serves as an indicator for selecting a few transformed cells introduced with a recombinant sequence from non-transformed cells. Examples of the marker gene include a reporter gene (e.g., a gene encoding a fluorescent protein, a gene encoding a luminescent protein, a gene encoding a protein that assists fluorescence, luminescence, or color development), a drug-resistant gene and the like. Only one type of marker gene may be used, or two or more types of marker gene (e.g., combination of a gene encoding a fluorescent protein and a drug-resistant gene) may be used. Use of two types or more can decrease false positivity more. As used herein, a protein encoded by a marker gene may be referred to as “marker protein”.

Examples of a fluorescent protein include, but are not limited to, a blue fluorescent protein such as Sirius, TagBFP, or EBFP; a cyan fluorescent protein such as mTurquoise, TagCFP, AmCyan, mTFP1, MidoriishiCyan, or CFP; a green fluorescent protein such as TurboGFP, AcGFP, TagGFP, Azami-Green (e.g., hmAG1), ZsGreen, EmGFP, EGFP, GFP2, or HyPer; a yellow fluorescent protein such as TagYFP, EYFP, Venus, YFP, PhiYFP, PhiYFP-m, TurboYFP, ZsYellow, or mBanana; an orange fluorescent protein such as KusabiraOrange (e.g., hmKO2), or mOrange; a red fluorescent protein such as TurboRFP, DsRed-Express, DsRed2, TagRFP, DsRed-Monomer, AsRed2, or mStrawberry; a near infrared fluorescent protein such as TurboFP602, mRFP1, JRed, KillerRed, mCherry, HcRed, KeimaRed (e.g., hdKeimaRed), mRasberry, or mPlum.

Examples of a luminescent protein include, but are not limited to, Aequorin. Further, examples of a protein that assists fluorescence, luminescence, or color development include, but are not limited to, an enzyme that decomposes a fluorescent, luminescent, or color development precursor such as a luciferase, phosphatase, peroxidase, or β-lactamase.

Examples of a drug-resistant gene include a drug-resistant gene such as a hygromycin-resistant gene (hygromycin phosphotransferase gene, hpt), a kanamycin-resistant gene, or a neomycin-resistant gene, and an herbicide-resistant gene such as an ALS (AHAS) gene or PPO gene. Among them, a hygromycin-resistant gene is preferred from the viewpoint of high selection efficiency in transformation using a rice callus.

Further, the nucleic acid of the present invention preferably comprises a negative marker gene in a region other than a recombinant sequence in order to remove cells in which a part or the whole of a region of the nucleic acid is unintentionally inserted to a region other than a target region. Thus, in one embodiment of the present invention, a nucleic acid comprising (1) a transposon of the present invention, (2) a homology region, and (3) a negative marker gene is provided. Examples of the above-described negative marker gene include, but are not limited to, diphtheria toxin protein A chain gene (DT-A), Exotoxin A gene, Ricin toxin A gene, codA gene, Cytochrome P-450 gene, RNase T1 gene and barnase gene.

A control region for causing the above-described marker gene and/or negative marker gene to express a protein encoded by the gene in a plant cell is operably linked to the nucleic acid of the present invention. When the protein is constantly expressed, examples of the control region include a promoter such as a cauliflower mosaic virus (CaMV) 35S promoter, a promoter of a nopaline synthase gene, a polyubiquitin 1 promoter derived from maize, an actin promoter derived from rice, or an elongation factor 1α promoter derived from rice, a terminator sequence for terminating transcription of a gene induced by the promoter or the like (heat-shock protein 17.3 terminator derived from rice, heat-shock protein 16.9a terminator derived from rice, actin terminator derived from rice, terminator of a nopaline synthase gene, terminator of an octopine synthase (OCS) gene, CaMV 35S terminator and the like), and a ΔEn terminator derived from a maize En/Spm transposon (En element) (Terada R. et al., Nat Biotechnol, 20(10):1030-1034 (2002)). Furthermore, an enhancer such as intron 1 of a castor bean Catalase gene, a CaMV 35S enhancer, a transcription enhancer E12, or an omega sequence also may be comprised in the control region in order to enhance the gene expression efficiency.

Further, it is preferable that an inducible promoter is operably linked to the nucleic acid of the present invention in order to cause a gene encoding the above-described transposase to transiently express the protein in a plant cell. Examples of the inducible promoter include a drug inducible promoter wherein expression of a transposase is induced by a drug, a stimulus inducible promoter wherein expression of a transposase is induced by a stimulus (e.g., a light inducible promoter wherein expression of a transposase is induced by a light stimulus, a heat-shock inducible promoter wherein expression of a transposase is induced by heat shock and the like) and the like. The inducible promoter is preferably a heat-shock inducible promoter. Further, the above-described terminator or enhancer may be connected to the nucleic acid of the present invention in order to control expression of a transposase.

A drug inducible promoter includes a TRE promoter (wherein expression of a protein is induced by contact with Doxycycline (Dox), tetracyline, or a derivative thereof or cancellation of the contact), a metallothionein promoter (wherein expression of a protein is induced by contact with a heavy metal ion), a steroid responsive promoter (wherein expression of a protein is induced by contact with a steroid hormone or a derivative thereof) and the like. Examples of a light inducible promoter include, but are not limited to, a ribulose-bisphosphate carboxylase small subunit gene (Rubisco) promoter (R. Fluhr et al., Proc. Natl. Acad. Sci. USA 83:2358, 1986), a promoter of a fructose-1,6-bisphosphatase gene (Japanese National Phase PCT Laid-Open Publication No. 7-501921), a promoter of a light harvesting chlorophyll a/b binding protein gene (Japanese Laid-Open Publication No. 5-89), and a LP2 leucine-rich repeat receptor kinase gene promoter of rice (Plant Biotechnology Journal (2009)7, pp. 1-16). Examples of a heat-shock inducible promoter include, but are not limited to, a promoter of a heat-shock protein (hsp) 16.9A gene derived from rice, a promoter of a hsp 16.9B gene, a promoter of a hsp 16.9C gene, a promoter of a hsp 18.2 gene derived from Arabidopsis thaliana, and a promoter of a hsp 70 gene derived from drosophila.

The nucleic acid of the present invention can be made by a method known per se. For example, it is possible to perform cloning by synthesizing, based on known DNA sequence information of a transposon, a transposase, a target region or the like, an oligo DNA primer to cover a desired part of the sequence and using a total RNA or an mRNA fraction prepared from a cell having the sequence as a template to perform amplification by RT-PCR method. Alternatively, it is also possible to chemically synthesize a DNA chain or connect a synthesized partially overlapping oligo DNA short chain using PCR method (overlapping PCR method) or Gibson Assembly method to thereby construct a DNA encoding the full length thereof. Construction of a full length DNA with chemical synthesis or combination with PCR method or Gibson Assembly method has an advantage in that a codon to be used can be designed over the CDS full length depending on the host introduced with the DNA. In expression of a heterologous DNA, converting the DNA sequence to a codon which is highly frequently used in a host organism can be expected to increase the protein expression level. For the data of the frequency of use of codons in hosts to be used, for example, the genetic code use frequency data base (http://www.kazusa.or.jp/codon/index.html) disclosed on the website of Kazusa DNA Research Institute can be used. Alternatively, documents containing the frequency of use of codons in each host may be referred to. With reference to obtained data and a DNA sequence which is intended to be introduced, a codon which is less frequently used in a host among the codons used for the DNA sequence may be converted to a codon which encodes the same amino acid and is highly frequently used. For example, when a host cell is a rice cell, a gene encoding a transposase optimized for codon use of monocotyledons such as rice or general angiosperms such as Arabidopsis thaliana can be used. For example, AcTPase4x having codon use suitable for expression in rice includes a DNA having the nucleotide sequence set forth in SEQ ID NO: 29 (the amino acid sequence is set forth in SEQ ID NO: 30).

The nucleic acid of the present invention may be in the form of a single-stranded DNA or may be in the form of a double-stranded DNA, and may be a linear DNA or may be a circular DNA. The nucleic acid of the present invention is preferably provided in the form of an expression vector placed under the control of the control region which is functional in a host cell. The nucleic acid of the present invention can be prepared in a suitable form depending on the type of the plant cell that is the subject of introduction.

An expression vector that can be replicated in a plant cell is not particularly limited as long as it has a replication origin (e.g., on of Ti plasmid, Ri plasmid or the like) that functions in the plant cell, but the expression vector preferably has a replication origin of E. coli (e.g., ColE1 on or the like) as well. When Agrobacterium method is used as a method for introducing a gene, it is generally necessary to further contain a T-DNA fragment from which a pathogenic gene of a Ti plasmid or a Ri plasmid is removed (including border sequences RB (right border) (e.g., the sequence set forth in SEQ ID NO: 31) and LB (left border) (e.g., the sequence set forth in SEQ ID NO: 32)). Examples of specific vectors include pBI-based vectors, pPZP-based vectors, or pSMA-based vectors and the like. Further, more suitable forms include binary vector-based vectors (pZHG, pKOD4, pBI121, pBI101, pBI101.2, pBI101.3, pBIG2113 and the like).

2. A Method for Producing Plant Cells with a Modified Genome

In another embodiment, the present invention provides a method for producing plant cells having a mutation in a target region of a double-stranded DNA by a two-stage selection step for plant cells introduced with the nucleic acid of the present invention of the above 1 (which may be hereinafter referred to as the “production method of the present invention”). The method comprises, for example: (1) the step of selecting plant cells introduced with the nucleic acid of the present invention; (2) the step of culturing the plant cells selected in step (1) under a condition wherein an inducible promoter is activated; and (3) the step of selecting a cell in which expression of a marker protein is eliminated from a population comprising the plant cells cultured in step (2). With this method, it is possible to introduce only a necessary mutation without leaving an unnecessary artificial DNA sequence such as an exogenous transposon or a gene encoding a transposase in the genome of the plant cells or while suppressing residual of the sequence as shown in the Examples described below. Thus, with the production method of the present invention, plant cells that have a mutation in a target region of a double-stranded DNA and do not have any of an exogenous transposon and a gene encoding a transposase, and further, more preferably, do not have a footprint caused by removal of the transposon, are produced. In other words, with the present invention, plant cells wherein no trace of a transposon is left in the genome are produced. That is, it is also possible to quickly modify the genome of plant cells without the need for a step of making a multiple transformant or performing backcross with a wild type in order to remove the exogenous factor as in the conventional techniques.

A plant from which cells to be introduced with the nucleic acid of the present invention are derived is not particularly limited, but is preferably a monocotyledon or a dicotyledon. Examples of a monocotyledon include a gramineous plant. The gramineous plant includes plants that belong to Oryza, Triticum, Hordeum, Secale, Saccharum, Sorghum, or Zea, which specifically include, but are not limited to, maize, sorghum, wheat, rice, oat, barley, rye, and millet. A preferable gramineous plant is maize, wheat, and rice. Wheat also includes wheat variety Nourin No. 61, from which a transformant was difficult to obtain with the conventional methods.

Examples of a dicotyledon include, but are not limited to, Brassicaceae plants, Leguminosae plants, Solanaceae plants, Cucurbitaceae plants, and Convolvulaceae plants. Brassicaceae plants include plants that belong to Raphanus, Brassica, Arabidopsis, Wasabia, or Capsella, which specifically include, but are not limited to, Brassica rapa var. pekinensis, rapeseed, cabbage, cauliflower, Raphanus sativus var. hortensis, Brassica rapa subsp. oleifera, Arabidopsis thaliana, Eutrema japonicum, and Capsella bursa-pastoris. A preferable Brassicaceae plant is Brassica rapa var. pekinensis and Capsella bursa-pastoris. Examples of Leguminosae plants include, but are not limited to, soy bean, Vigna angularis, Phaseolus vulgaris, and Vigna unguiculata. A preferable Leguminosae plant is soy bean. Examples of Solanaceae plants include, but are not limited to, tomato, eggplant, and potato. A preferable Solanaceae plant is tomato. Examples of Cucurbitaceae plants include, but are not limited to, oriental melon, cucumber, melon, and watermelon. A preferable Cucurbitaceae plant is oriental melon. Examples of Convolvulaceae plants include, but are not limited to, Ipomoea nil, Ipomoea batatas, and Calystegia japonica. A preferable Convolvulaceae plant is Ipomoea batatas.

Apart from the above plants, the examples of the plants further include plants of Rosaceae, Lamiaceae, Liliaceae, Chenopodiaceae, Apiaceae, Asteraceae and the like. Furthermore, any tree species, any fruit tree species, Moraceae plants (e.g., rubber), and Malvaceae plants (e.g., cotton) are included.

As used herein, a “plant body” encompasses all of a plant individual, a plant organ, a plant tissue, a plant cell, and a seed. Examples of a plant organ include a root, a leaf, a stem, and a flower and the like. Further, a plant cell also includes a cell in a plant body in addition to a cultured cell. Furthermore, a plant cell in various forms (e.g., a suspension cultured cell, a protoplast, a section of a leaf, a section of a root, a callus, an immature embryo, pollen and the like) is included.

The production method of the present invention may comprise the step of introducing the nucleic acid of the present invention to plant cells prior to the above-described step (1). Introduction of the nucleic acid of the present invention (e.g., a nucleic acid in the form of an expression vector) can be implemented for appropriate tissue (e.g., a callus, a root, a leaf, a seed, a growing point or the like) depending on the type of the plant cells in accordance with a known method (e.g., Agrobacterium method, PEG method, electroporation method, particle gun method or the like). For example, in the case of rice, Agrobacterium method, whisker direct introduction method or the like is generally used, but the used method is not limited thereto. For example, in the case of Agrobacterium method, a callus is induced from a rice seed in accordance with a common method, the callus is infected with Agrobacterium introduced with a T-DNA fragment of a vector for Agrobacterium expression which is incorporated with a part or the whole of the region of the nucleic acid of the present invention, and the bacterium is removed after a few days (e.g., three days). Meanwhile, in the case of whisker direct introduction method, an expression vector is mixed with polyornithine to form a complex, the complex is then added to a rice callus together with potassium titanate whiskers and mixed, followed by ultrasonic treatment.

In the case of wheat or maize, for example, an expression vector can be introduced by using Agrobacterium method in the same manner, with an immature embryo collected from an immature seed as a plant material.

When PEG method or electroporation method is used, a protoplast is prepared from an appropriate cell/tissue in accordance with a common method, and an expression vector is introduced to the protoplast. In the case of particle gun method, an expression vector adsorbed to a gold microparticle can be introduced to a callus, an immature embryo, a growing point present in a shoot apex or an axillary bud or the like by using a particle gun.

With particle gun method or Agrobacterium method, gene introduction often results in a chimera. Thus, sample cells wherein the above-described nucleic acid is highly frequently introduced to germ line cells need to be used for transformation. For example, an embryo, a hypocotyl section, an embryogenic callus, an isolated growing point and the like are included.

Culture of plant cells introduced with the nucleic acid of the present invention can be implemented in accordance with a known method depending on the type. A preferable medium used for culture is a solid medium (e.g., an agar medium, an agarose medium, a gellan gum medium or the like). Further, a medium preferably contains a carbon source, a nitrogen source, an inorganic substance or the like which is necessary for growth of a transformant. For example, an N6 medium, an MS medium, an LS medium, a B5 medium or the like is used as a basal medium. A plant growth substance (e.g., auxins, cytokinins or the like) or the like may be appropriately added to a medium. PH of a medium is preferably about 5 to about 8. Culture temperature can be appropriately selected within the range of about 20° C. to about 35° C. in general depending on the type of the plant cells. For example, a rice callus can be generally cultured at 28 to 33° C., and preferably at 30 to 33° C.

Those skilled in the art can perform step (1) of the production method of the present invention through appropriate selection by a known method depending on the type of the marker gene that is used. For example, when a drug-resistant gene is used, plant cells in which a mutation or the like is introduced to a target region can be selected by culturing plant cells introduced with the nucleic acid of the present invention in the presence of a corresponding drug. For example, when hygromycin is used as a drug, the concentration thereof in a medium is preferably 10 mg/L to 200 mg/L (e.g., 50 mg/L).

When a reporter gene is used, plant cells in which a mutation or the like is introduced to a target region can be selected by detecting a signal from a reporter protein encoded by the gene using a predetermined detection apparatus. The detection apparatus includes, but is not limited to, a flow cytometer, an imaging cytometer, a fluorescence microscope, a luminescence microscope, a CCD camera and the like. Those skilled in the art can use a suitable apparatus as said detection apparatus depending on the type of the reporter protein. For example, when the reporter protein is a fluorescent protein or a luminescent protein, selection is possible using a flow cytometer. When the reporter protein is a protein that assists fluorescence, luminescence, or color development, selection is possible by using a microscope, using a culture dish coated with a photoresponsive cell culture substrate, irradiating plant cells subjected to color development or the like with light, and utilizing the cells that were not irradiated being peeled from the culture dish.

Further, after performing the step of selection using expression of the marker protein as an indicator, PCR method, sequencing method, Southern blotting method, CAPS (cleaved amplified polymorphic sequence) method or the like may be used to confirm that a transposon has been introduced to a target region of plant cells by homologous recombination.

The “condition wherein an inducible promoter is activated” in step (2) of the production method of the present invention means a condition wherein, when the inducible promoter is a drug inducible promoter, continuous culture of plant cells in the presence of a substance described in the above 1. corresponding to each drug inducible promoter or continuous culture of plant cells in the absence of a substance that inhibits activation of the promoter activates the promoter to thereby induce expression of a transposase. When the inducible promoter is a stimulus inducible promoter, the above condition means a condition wherein culture of plant cells under a stimulus (e.g., light or heat) corresponding to each promoter activates the promoter to thereby induce expression of a transposase.

The culture time of step (2) is not particularly limited as long as a transposon is removed within the time, but the culture time is preferably 5 minutes or more (e.g., 5 minutes, 10 minutes or more). Further, the culture time is preferably 120 minutes or less (e.g., 120 minutes, 90 minutes or less) in order to suppress damage to plant cells due to drugs or stimuli. In a preferred embodiment, the time of step (1) is 40 minutes to 60 minutes, particularly preferably 40 minutes. When a drug response inducible vector is used, the concentration of a drug in a medium in step (1) is not particularly limited as long as a transposase is expressed in cells. For example, when Dox is used, about 0.4 μg/mL to 1.5 μg/mL is preferred. When a drug other than Dox is used, those skilled in the art can appropriately set the concentration. The culture temperature of when a heat-shock response inducible vector is used is not particularly limited as long as expression of a transposase is induced, but is preferably 32° C. or greater (e.g., 35° C., 36° C., 37° C., 38° C., 39° C., 40° C. or greater). Further, 47° C. or below (e.g., 46° C., 45° C., 44° C., 43° C. or below) is preferred in order to suppress damage to plant cells due to heat. In a preferred embodiment, the culture temperature is 42° C. The present inventors found that when a heat-shock responsive vector is used, a removal unit can be removed without applying a heat-shock stimulus in some cases (data not shown). Thus, even when a heat-shock responsive vector is used, cells may be cultured at normal culture temperature (e.g., about 20° C. to about 35° C.).

In step (3) of the production method of the present invention, when expression of a marker protein cannot be detected or when the expression level of a marker protein lowers to a background level, it can be evaluated that expression of the marker protein is eliminated. Detection of expression of a marker protein or measurement of the expression level can be appropriately performed by using, for example, the above-described detection apparatus.

Further, after performing the step of selection using expression of the marker protein as an indicator, PCR method, sequencing method, Southern blotting method, CAPS (cleaved amplified polymorphic sequence) method or the like may be used to confirm that a transposon has been removed from a target region.

3. A Plant Cell with a Modified Genome or a Plant Body Comprising the Cell

In another embodiment of the present invention, a plant cell (which may be hereinafter referred to as the “plant cell of the present invention”) obtained by the production method of the present invention of the above 2. or a plant body comprising the cell is provided. A plant body comprising the plant cell of the present invention can be obtained by regenerating the cell. Since the plant cell of the present invention has a desired mutation in a target region, the phenotype of a plant body regenerated from such a plant cell can vary in association with the mutation. Thus, it is possible to efficiently analyze the function and the like of the target region by using the plant cell of the present invention. The definition of plant cell and a plant body, the type of plants and the like are as described in the above 2.

A transformant clone confirmed to be introduced with a mutation, which is obtained by the production method of the present invention, can be regenerated into a plant body by a regeneration method known per se. In the case of rice, examples of a regeneration method include a method described in Toki S. et al., Plant Physiol. 100(3):1503-1507 (1992), a method described in Christou P. et al., Bio/Technology 9:957-962 (1991), a method described in Hiei Y. et al., Plant J., 6: 271-282, (1994) and the like.

Once a plant body comprising a cell in which a mutation is introduced to a target region is obtained in this manner, it is possible to obtain progeny from the plant body by sexual reproduction or asexual reproduction. Further, it is also possible to obtain a propagation material (e.g., seeds, fruits, cuttings, stubbles, calli, protoplasts or the like) from the plant body or progeny or clones thereof and mass-produce the plant body based on the propagation material. Thus, the present invention includes a plant body comprising the plant cell of the present invention, progeny and clones of the plant body, and a propagation material of the plant body and progeny and clones thereof. When a mutation is introduced to heterozygosity, an R1 plant obtained by self-pollination of a resulting plant body is further subjected to self-pollination to obtain an R2 plant, whereby a plant body in which a mutation is introduced to homozygosity can be obtained.

### 4. A Method for Modifying the Genome of a Plant Cell

In another embodiment, the present invention provides a method for introducing a mutation to a target region of a double-stranded DNA of a plant cell for plant cells introduced with the nucleic acid of the present invention of above 1. The method comprises, for example: (1) the step of selecting plant cells introduced with the nucleic acid of the present invention; and (2) the step of culturing the plant cells selected in step (1) (preferably culturing the plant cells under a condition wherein an inducible promoter is activated), and may optionally comprise (3) the step of selecting a cell in which expression of a marker protein is eliminated from a population comprising the plant cells cultured in step (2). With this method, it is possible to introduce only a necessary mutation without leaving an unnecessary artificial DNA sequence such as an exogenous transposon or a gene encoding a transposase in the genome of the plant cells or while suppressing residual of the sequence. Thus, with the above-described method, only a mutation of interest can be introduced to the genome of a plant cell without introducing any of a plant cell exogenous transposon and a gene encoding a transposase, more preferably, and further a footprint caused by removal of the transposon. In other words, the present invention provides a method for modifying the genome of a plant cell wherein a trace of a transposon is not left in the genome. The above-described steps (1) to (3) can be performed in the same manner as the method described in above 2.

The present invention is described hereinafter based on Examples. However, the present invention is not limited to these Examples.

### EXAMPLES

Experiments were conducted in the following manner in the Examples described below.

### <Plant Materials and Growth Conditions>

Japonica rice (Oryza sativa L.) (cultivar: “Nipponbare” and “Kinmaze”) was used in the Examples. Rice was grown in a green house under long day conditions (16 hours:8 hours, light:dark, 28° C.:22° C.).

### <Construction of a Marker Excision System>

A DNA transposon Ac/Ds (Activator/Dissociation) system of artificially manipulated maize was used for heat-shock inducible marker excision. The DNA sequence (Muller-Neumann M. et al., Mol. Gen. Genet, 198(1):19-24 (1984)) of an Ac element isolated from a wx-m7 allele of maize was referred to.

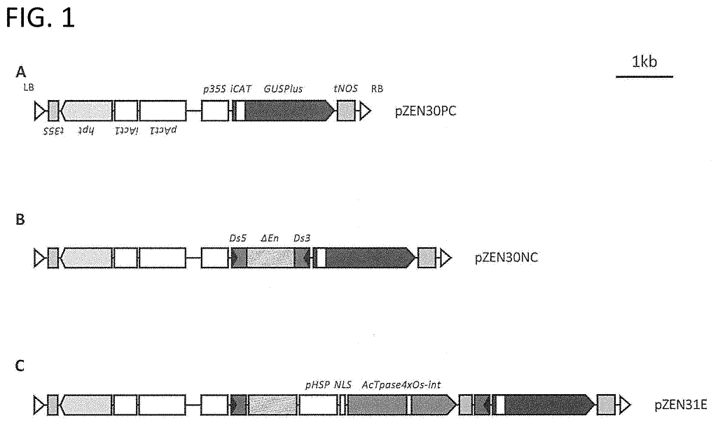

An expression cassette of inducible-type AcTPase was synthesized based on the following design. An AcTPase gene of rice (AcTPase₄ₓOs) comprises a codon-optimized ORF (the sequence set forth in SEQ ID NO: 47) of enhanced AcTPase (Lazarow K. et al., Genetics 191:747-756 (2012)) in which a nuclear localization signal of an SV40 large T antigen and intron 2 of a wild-type AcTPase gene in a corresponding position are fused. A region adjacent to 5′ of Oshsp16.9C (Chang P. L. et al., Botanical Bulletin-Academia Sinica Taipei, 42(2):85-92 (2001), Guan J. C. et al., Plant Mol Biol, 56(5):795-809 (2004), Itoh H. et al., Nat Genet, 42(7):635-638 (2010)) was used as a heat-shock promoter (the sequence set forth in SEQ ID NO: 46). AcTPase₄xOs was placed between the heat-shock promoter of rice and the NOS terminator (the sequence set forth in SEQ ID No: 48) (FIG. 1C).

An artificial Ds element was designed to have ΔEn (Terada R. et al., Nat Biotechnol, 20(10):1030-1034 (2002)) (the sequence set forth in SEQ ID No: 45) that is a transcription terminator in a region within 300 bp from both terminals of the Ac element. This terminal region comprises a terminal inverted repeat (TIR) and an adjacent subterminal region having an AcTPase binding motif essential for making transposition of the Ac/Ds element efficient (Becker H. A. and Kunze R., Mol Gen Genet. 254(3):219-230 (1997)).

Binary vectors for a GUS reporter assay were made based on pZEN11 (Shimatani Z. et al., Mol Genet Genomics. 281(3):329-344 (2009)). In order to construct pZEN30NC, a Ds element was inserted to the downstream of a p35S promoter that prevents expression of GUSPlus (FIG. 1B). In pZEN31E, an inducible AcTPase cassette was inserted adjacently to the ΔEn element of pZEN30NC (FIG. 1C). Further, a vector that imitates the result of Ds excision was constructed as pZEN30PC (FIG. 1A).

### <Evaluation of an Ac/Ds-Mediated Marker Excision System>

Similarly to a prior report (Shimatani Z. et al., Mol Genet Genomics. 281(3):329-344 (2009)), binary vectors, i.e., pZEN30PC, pZEN30NC, and pZEN31E, were introduced to proliferative rice calli derived from a germ disc of “Nipponbare”. After selection for four weeks, hygromycin-resistant callus lines were used for evaluation of an inducible Ac/Ds-mediated marker excision system. Each callus line was divided into two copies, wherein one of them was cultured at normal temperature (31.5° C.) while the other was exposed to high temperature of 42° C. for 90 minutes. After subculture for one week, excision of the artificial Ds element was studied by histochemical GUS reporter assay and PCR analysis in the same manner as the above description (Shimatani Z. et al., Mol Genet Genomics. 281(3):329-344 (2009)). In order to determine a footprint generated by Ds excision, the amplification product of PCR was cloned into pCR-BlutnII-TOPO (Thermo Fisher Scientific) and analyzed by Sanger sequencing.

### <A Gene Targeting Vector Having an Autonomous Marker Excision System>

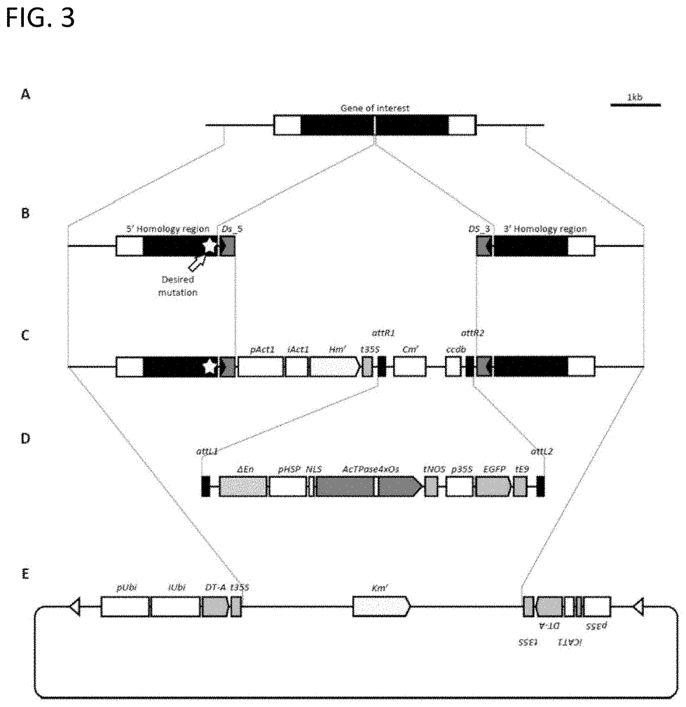

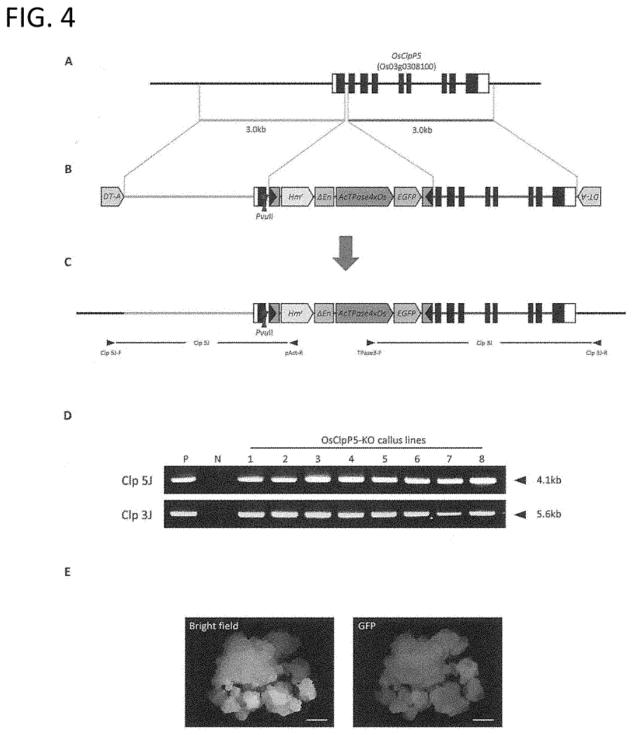

Modules as shown in FIG. 3 and FIG. 4 were combined to construct a gene targeting vector having an autonomous marker removal system. A homology region for homologous recombination (HR) was made by PCR using a Tks Gflex DNA polymerase (Takara Bio) and an appropriate primer listed in Table 1 and Table 2.

In order to construct vectors pOsClpP5KO-AcDs and pOsClpP5KO-Ds, 3 kb (the sequence set forth in SEQ ID NO: 42) of a 5′ homology arm comprising a promoter region of OsClpP5 was prepared from the genome of “Nipponbare” by nested PCR, which was then fused with the 5′ terminal region of a Ds element by overlapping PCR method (FIG. 3B). A PvuII site that results in a KO mutation was introduced with this process (FIG. 4B). Similarly, 3 kb (the sequence set forth in SEQ ID NO: 50) of a 3′ homology arm comprising a sequence encoding OsClpP5 was amplified and fused to the 3′ terminal region of the Ds element. Similarly, 3.55 kb and 3.66 kb fragments were cloned to construct pOsClpP5KO-Con5 and pOsClpP5KO-Con3, respectively, which are positive control vectors. These two vectors were used to make true 5′ and 3′ junction fragments in PCR screening of gene-targeted (GT) candidates. The full length sequence of the made targeting vector pOsClpP5KO-AcDs is indicated as SEQ ID NO: 36. The sequence of pOsClpP5KO-Ds corresponds to a sequence in which the region of position 8626 to position 9394 (the sequence of a heat-shock promoter), the region of position 9411 to position 11626 (the sequence of an AcTPase4x gene comprising an intron and an SV40 NLS), and the region of position 11665 to position 11917 (the sequence of an NOS terminator) are removed from SEQ ID NO: 36.

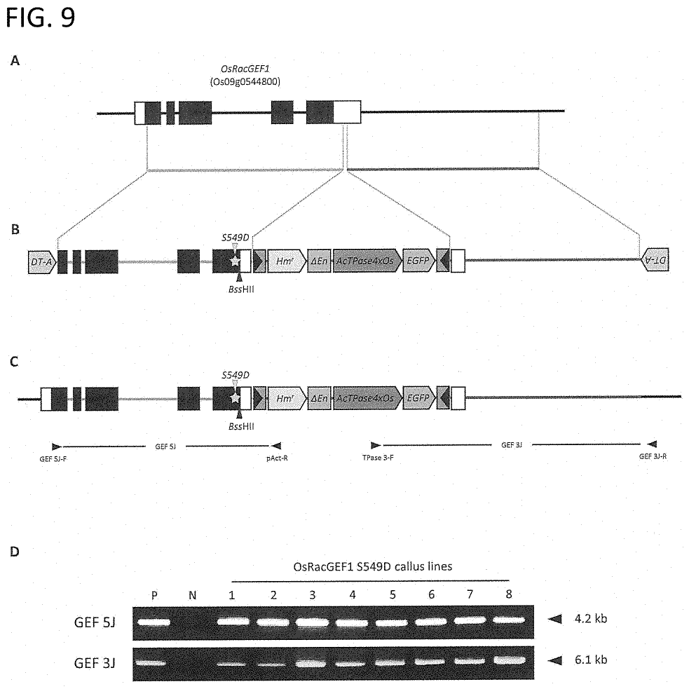

For vectors (pGEF1S549D-AcDs and pGEF1S549D-Ds) to introduce an S549D mutation to an OsRacGEF1 gene, 3 kb and 3.24 kb fragments were amplified from the “Kinmaze” genome for 5′ and 3′ homology arms, respectively. The terminal region of Ds was then fused by overlapping PCR (FIG. 9B). In this step, an S549D mutation and an adjacent BssHII site were introduced to the 5′ homology arm. pGEF1S549D-Con, which is a control vector having true 5′ and 3′ junction fragments, was also constructed. The sequence of the made targeting vector pGEF1S549D-AcDs corresponds to a sequence in which the region of position 1593 to position 4592 (the sequence of a 5′ homology arm) is substituted with the sequence set forth in SEQ ID NO: 155 and the region of position 13915 to position 16912 (the sequence of a 3′ homology arm) is substituted with the sequence set forth in SEQ ID NO: 156 in SEQ ID NO: 36. The sequence of pGEF1S549D-Ds corresponds to a sequence in which the region of position 8626 to position 9394 (the sequence of a heat-shock promoter), the region of position 9411 to position 11626 (the sequence of an AcTPase4x gene comprising an intron and an SV40 NLS), and the region of position 11665 to position 11917 (the sequence of an NOS terminator) are removed from the sequence of pGEF1S549D-AcDs.

A DNA fragment comprising inducible AcTPase4xOs and a gene of a fluorescent protein was incorporated by site-specific recombination using LR clonaseII (Thermo Fisher Scientific) (FIG. 3D).

The DNA sequence of the homology arms was confirmed by Sanger sequencing using primers listed in Tables 1 and 2.

TABLE 1
List of primers for PCR cloning, PCR analysis and DNA sequencing analyses (OsClpPS KO)

| | | | | |
|---|---|---|---|---|
| No. | Designation | Sequences (5′→3′)^{a} | SEQ ID NO | Comments |

5′ Homology arms cloning

| | | | | |
|---|---|---|---|---|
| 1 | ClpP5 5 1st-F | GCTCTCCGCGTTTTGGGTACAGGCAATTCAGACAG | 51 | 1st PCR of 5′ homology region cloning |
| 2 | ClpP5 5 1st-R | acCTGATCGACCAAATCCCGCTCCTCTGAT | 52 | |
| 3 | ClpP5 5-F | AGTCATTTAAATCGTCGACGTGACTGTTTAGTGAAAATAACTCATGAAAT | 53 | 2nd PCR of 5′ homology region |
| | | SwaI: ATTTAAAT, SgrDI: CGTCGACG | | cloning |
| 4 | ClpP5-Ds 5-R | cggaagaggggagtgcagCTGAGGAGCGGCAGAGATCTCGGGGCGGGGTTCG | 54 | |
| 5 | ClpP5-ds 5-F | GCCGCTCCTCAGctgcactcccctcttccgCAGGGATGAAAGTAGGATGGGAAAATCC | 55 | 5′ terminal region of Ds element |
| 6 | Ds 5-R | TGACGCGGCCGCTTCTACATGGGCTGGGCCTCAGTGGTTAT | 56 | |
| | | Noti: GCGGCCGC | | |
| 7 | ClpP5 5Con-F | AGTCATTTAAATCGTCGACGTATTACTGTTATTCCTGCATGCAGTGGAAG | 57 | 2nd PCR of 5′ homology region |
| | | | | cloning for control vector |

3′ Homology arms cloning

| | | | | |
|---|---|---|---|---|
| 8 | ClpP5 3 1st-F | CCCTCGCCCAAAACCCCCACGAGCCGCGGC | 58 | 1st PCR of 3′ homology region cloning |
| 9 | ClpP5 3 lst-R | GCTATACTACCCCATTCGGTGTCTGAGCGG | 59 | |
| 10 | Ds 3-ClpP5 3-F | ACCGACCGTTACCGACCGTTTTCATCCCCTAgctccctcctcactctactctgtcgtggct | 60 | 2nd PCR of 3′ homology region |
| 11 | ClpP5 3-R | TGACGGCGCGCCTTTCATCATATTATAAATCGTTTTAATTTTTTTATAG | 61 | cloning |
| | | AscI: GGCGCGCC | | |
| 12 | Ds 3-F | AGTCCCTCGAGGTGCTCCAGATTTATATGGATTTTATCTATG | 62 | 3′ terminal region of Ds element |
| | | AbsI: CCTCGAGG | | |
| 13 | Ds 3-ClpP5 3-R | agccacgacagagtagagtgaggagggagcTAGGGATGAAAACGGTCGGTAACG | 63 | |
| | | GTCGGT | | |
| 14 | ClpP5 3Con-R | TGACGGCGCGCCTGCAGCTACGCACTACTGTTAATCAAAGTT | 64 | 2nd PCR of 3′ homology region |
| | | | | cloning for control vector |

Screening of GT transformants

| | | | | |
|---|---|---|---|---|
| 15 | Clp 5J-F | AGGTGCTTTACATCTTGCAATGTAAGTACC | 65 | 5′ side junction PCR |
| 16 | pAct-R | GTCCAGTTGGCTGGACTGGGGTGGGTTGGG | 66 | |
| 17 | TPase 3-F | CACCAACCACGAGAACCACATGGACGAGGA | 67 | 3′ side junction PCR |
| 18 | Clp 3J No.2-R | TTCTCCCACAGTCCCACTAATGTGAAATGA | 68 | |

Detection of marker gene

| | | | |
|---|---|---|---|
| 19 | En No2-F | CATGCTAGTCCGCCTCCGGATCAGAGCACG | 69 |
| 20 | pHSP J-R | GAGCCATGGCCAAGGTTCTGCAGGAGCTCG | 70 |

CAPS analysis

| | | | | |
|---|---|---|---|---|
| 21 | Clp Ex F-G | AGTCAAGCTTTGTGTCTGGTGGACGTCCGTGGTGCGCGCG | 71 | PCR for CAPS analysis with PvuII |
| | | HindIII: AAGCTT | | digestion |
| 22 | Clp Ex-R | ATCCGAATTCGCTGGGCAACGATGATGTTCGCCATATCATCCTCC | 72 | |
| | | EcoRI: GAATTC | | |

DNA seguencing analysis

| | | | |
|---|---|---|---|
| 23 | ClpP5 F-1 | GGACAAAACTGGGGATTAATGATCAGCGAG | 73 |
| 24 | ClpP5 F-2 | GTAGATGGTGGGTTGCATGAGTCACCAATT | 74 |
| 25 | ClpP5 F-3 | TTGTCAATGGCCATGACTCTACGCCAGACA | 75 |
| 26 | ClpP5 F-4 | CTGTGGGGGACATGTTCGTCGACCTCCGGT | 76 |
| 27 | ClpP5 F-5 | CTTGACACTTTTGTATTGAGTTTCGCTGTA | 77 |
| 28 | ClpP5 F-6 | TGTGTCTGGTGGACGTCCGTGGTGCGCGCG | 78 |
| 29 | ClpP5 F-7 | TTGGGTGGGGATGCGAGGGAGCTGATGATG | 79 |
| 30 | ClpP5 F-8 | CTTGTTTTAATCGATGATTGTTGTTAATCC | 80 |
| 31 | ClpP5 F-9 | CCAGTTGTATATCCCGCCTAATTCCTTCTC | 81 |
| 32 | ClpP5 F-10 | TGAATGTAACACAGGGAAGCGATACAGCTT | 82 |
| 33 | ClpP5 F-11 | CCCTTCAACCGCTTCCTGCTTCTAGTTAGC | 83 |
| 34 | ClpP5 F-12 | GCAACTTATTTGATACTCCCTCTGTTTCAT | 84 |
| 35 | ClpP5 R-1 | AAATTTGATCAAGTATATAAAAAATATAGC | 85 |
| 36 | ClpP5 R-2 | TTAGCCTTGTGATGCAGCATCTCATTAGCC | 86 |
| 37 | ClpP5 R-3 | CTGGAATACTGATAAGCACTCCTGAATTTG | 87 |
| 38 | ClpP5 R-4 | CACTGATCCTCCAGGAGAATTCACATACAT | 88 |
| 39 | ClpP5 R-5 | AGATATAGCAGCTGGGCAACGATGATGTTC | 89 |
| 40 | ClpP5 R-6 | CCGCGTTCCTGCCCCGACGCATACTCCACA | 90 |
| 41 | ClpP5 R-7 | GAGAATAGTGTGTGTCTTCTTCGAGACTCG | 91 |
| 42 | ClpP5 R-8 | CCCTAAATTTTAAGCGTCACTTTGAATCGC | 92 |
| 43 | ClpP5 R-9 | CTACATCCTAGCACTACTAAACATGGTTAG | 93 |
| 44 | ClpP5 R-10 | CCATAATATATTTGTAGCCGAATCCACCAC | 94 |
| 45 | ClpP5 R-11 | CAATAATTTTCATAATGGTAAGGTACAGTT | 95 |
| 46 | ClpP5 R-12 | TGCTGTCAACAGCATGTGACCTCAATAAGA | 96 |

TABLE 2
List of primers for PCR cloning, PCR analysis and DNA sequencing analyses (OsRacGEF1 S549D)

| | | | | |
|---|---|---|---|---|
| No. | Designation | Sequences (5′→3′)^{a} | SEQ ID NO | Comments |

5′ Homology arms cloning

| | | | | |
|---|---|---|---|---|
| 1 | GEF1 5′ 1st-F | GAGCAGCCGCATCAGTAGTCATCAGTGTGC | 97 | 1st PCR of 5′ homology region cloning |
| 2 | GEF1 5′ 1st-R | TCCCTTACTTGTTGTAAGTAGGGCTAGACG | 98 | |
| 3 | GEF1 5-F | AGTCATTTAAATCGTCGACGTCCGCCAAGCCCGCCGCCGACCTCTCCGg | 99 | 2nd PCR of 5′ homology region |
| | | SwaI: ATTTAAAT, SgrDI: CGTCGACG | | cloning |
| 4 | GEF1-Ds 5-R | AGTCTCTTTCAGGGGCATCTCCTGCGTCTTTGCGCGCGTCAAGGTTCCCG | 100 | |
| | | GCATACGTCC | | |
| | | BssHII: GCGCGC, S549D mutation: GTC | | |
| 5 | GEF1-ds 5-F | GCAGGAGATGCCCCTGAAAGAGACTGAAAGGCAGTGAAGAAAGTTGC | 101 | 5′ terminal region of Ds element |
| | | AGGGATGAAAGTAGGATGGGAAAATCCCG | | |
| 6 | Ds 5-R | TGACGCGGCCGCTTCTACATGGGCTGGGCCTCAGTGGTTAT | 56 | |
| | | Noti: GCGGCCGC | | |
| 7 | GEF1 5Con-F | AGTCATTTAAATCGTCGACGTATTACTGTTATTCCTGCATGCAGTGGAAG | 102 | 2nd PCR of 5′ homology region |
| | | | | cloning for control vector |

3′ Homology arms cloning

| | | | | |
|---|---|---|---|---|
| 8 | GEF1 3′ 1st-F | CTTTCTGCTCCTCCACTCCAGTTACAGGCA | 103 | 1st PCR of 3′ homology region cloning |
| 9 | GEF1 3′ lst-R | GGTGGGACAGCCGTTGAGGTAGGGCCGAGA | 104 | |
| 10 | Ds 3-GEF1F | CCGACCGTTACCGACCGTTTTCATCCCTAaTCAGATCTTTAGACGTGTCTC | 105 | 2nd PCR of 3′ homology region |
| | | AGCAAGGTC | | cloning |
| 11 | GEF1 3-R | TGACGGCGCGCCTTTCATCATATTATAAATCGTTTTAATTTTTTTATAG | 106 | |
| | | AscI: GGCGCGCC | | |
| 12 | Ds 3-F | AGTCCCTCGAGGTGCTCCAGATTTATATGGATTTTATCTATG | 62 | 3′ terminal region of Ds element |
| | | AbsI: CCTCGAGG | | |
| 13 | Ds 3-GEF1-R | ACCTTGCTGAGACACGTCTAAAGATCTGAtTAGGGATGAAAACGGTCGG | 107 | |
| | | TAACGGTCGGT | | |
| 14 | GEF1 3Con-R | TGACGGCGCGCCGCCATAATTTAATATGTGTGTGAGACTTCTCTTTC | 108 | 2nd PCR of 3′ homology region |
| | | | | cloning for control vector |

Screening of GT transformants

| | | | | |
|---|---|---|---|---|
| 15 | GEF 5J-F | CCGCCGCGACTAGCTAGCTAGCTCGGAGGT | 109 | 5′ side junction PCR |
| 16 | pAct-R | GTCCAGTTGGCTGGACTGGGGTGGGTTGGG | 66 | |
| 17 | TPase 3-F | CACCAACCACGAGAACCACATGGACGAGGA | 67 | 3′ side junction PCR |
| 18 | GEF 3J-R | TGCTAGAAACATGGATGTCTAGAGTGCATG | 110 | |

Detection of marker gene

| | | | |
|---|---|---|---|
| 19 | En No2-F | CATGCTAGTCCGCCTCCGGATCAGAGCACG | 69 |
| 20 | pHSP J-R | GAGCCATGGCCAAGGTTCTGCAGGAGCTCG | 70 |

CAPS analysis

| | | | | |
|---|---|---|---|---|
| 21 | GEF Ex F-S | AGTCAAGCTTGACGTTGGGCAGTCCATACTAGAGAGTTAC | 111 | PCR for CAPS analysis with BssHII |
| | | HindIII: AAGCTT | | digestion |
| 22 | GEF Ex-R | ATCCGAATTCTATTGGTGATGGAAATTATAGTCAAGGAGGATCACC | 112 | |
| | | EcoRI: GAATTC | | |

DNA seguencing analysis

| | | | |
|---|---|---|---|
| 23 | GEF1 F-1 | TTTCCAGAGGTGGACATGATGAAGGAGCGC | 113 |
| 24 | GEF1 F-2 | AGGCGGCCATGGCGATTAACAGCGATGTGC | 114 |
| 25 | GEF1 F-3 | CATTCTTACCAATTATCAGTGGAAGCTGAC | 115 |
| 26 | GEF1 F-4 | GAGGCTGCCATCCATGTGTGGAGGTTGAAG | 116 |
| 27 | GEF1 F-5 | GACGTTGGGCAGTCCATACTAGAGAGTTAC | 117 |
| 28 | GEF1 F-6 | AGCCAAGGGTCGTCTAGCCCTACTTACAAC | 118 |
| 29 | GEF1 F-7 | CCTAATGGACAGATCTGACGCTCTTCAGTG | 119 |
| 30 | GEF1 F-8 | CGAAGGCCGCCGGGCCGCAGCTGAAGCTGC | 120 |
| 31 | GEF1 F-9 | GCATCGAGCCCATGTCCACCATCTCCGCCG | 121 |
| 32 | GEF1 F-10 | CTGCTGAAGGCGTCTCTGGAGTCGACGACG | 122 |
| 33 | GEF1 F-11 | TGTAACCCGGTCGAAGCTTCACCCAATGCA | 123 |
| 34 | GEF1 F-12 | CCCTGGATCAAGTAGTCTGGTACACACGAA | 124 |
| 35 | GEF1 R-1 | ATATCTCTCTGAGTTCCACACATAGTAACC | 125 |
| 36 | GEF1 R-2 | TGCACCCACACACAGCTAGAGCACTGACAC | 126 |
| 37 | GEF1 R-3 | ATCAACATATTGTCACCATCTCAGTTCATC | 127 |
| 38 | GEF1 R-4 | AAAATCCACTCCCTCCGGATAGAGAAGGGT | 128 |
| 39 | GEF1 R-5 | AGGAAGAAGGCGTCGTCGAGGGCGCGCGCG | 129 |
| 40 | GEF1 R-6 | GCTAAGCCTAAGCTAATAGCAGAGGCAACC | 130 |
| 41 | GEF1 R-7 | GATTGAAGATGGAAACAGAGTCAGCAGCGG | 131 |
| 42 | GEF1 R-8 | CAACAACCCATCAGCTCTTTGGGACAAAAC | 132 |
| 43 | GEF1 R-9 | GGTTACTATGTGTGGAACTCAGAGAGATAT | 133 |
| 44 | GEF1 R-10 | GTCAGCTTCCACTGATAATTGGTAAGAATG | 134 |
| 45 | GEF1 R-11 | CTTTGGCAGTGACTCCAAGTACACTTCCGG | 135 |
| 46 | GEF1 R-12 | CCAGCCTCCACAGCTCGCCGAACACAGTGG | 136 |
| 47 | GEF1 R-N2 | TGCCTGTAACTGGAGTGGAGGAGCAGAAAG | 137 |

### <Transformation of Rice for Gene Targeting>

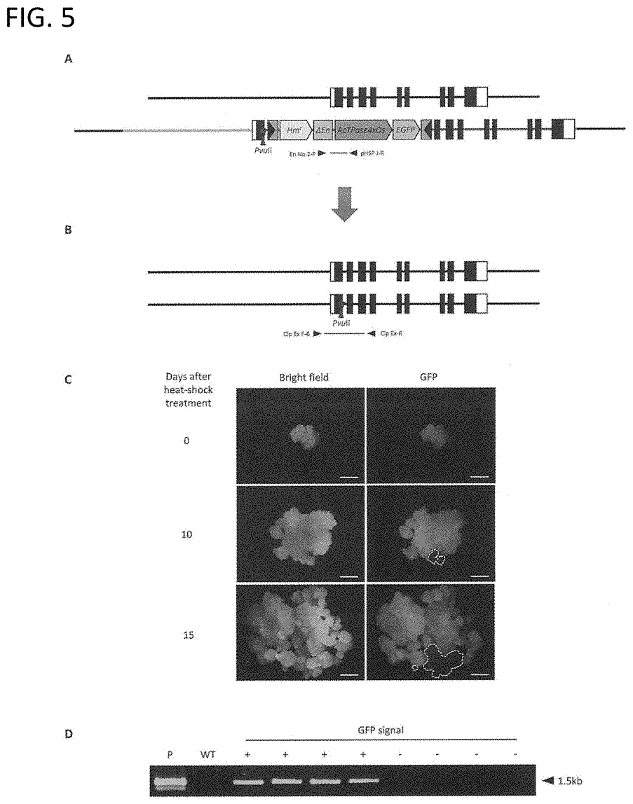

“Nipponbare” and “Kinmaze” were used to knock out (KO) OsClpP5 and to introduce an S549D mutation to OsRacGEF1, respectively. Transformation mediated by large-scale Agrobacterium was basically performed according to prior reports (Terada R. et al., Nat Biotechnol, 20(10):1030-1034 (2002), Terada R. et al., Plant Physiol, 144(2):846-856 (2007)), but the following change was added. Germ disc-derived embryogenic calli were induced from 250 to 500 mature seeds on an N6D medium. In this case, embryogenic calli means calli that maintain a high regeneration potency. After Agrobacterium was removed, rice calli were cultured in an N6DNU medium for ten days to ensure that the Agrobacterium was removed. The rice calli were then transferred to an N6DSE-H40 medium and GT candidates were selected by positive-negative selection. Proliferative callus lines were subjected to PCR screening using an appropriate primer (Tables 1 and 2, FIGS. 5B and 9C) to identify GT callus lines. The selected callus lines were individually subcultured and subjected to heat-shock treatment at 42° C. for 40, 60, or 90 minutes, thereby inducing Ac/Ds-mediated marker excision. As described below, introduction of a desired mutation and excision of a marker in the callus lines were confirmed by PCR and DNA sequencing analysis. The selected callus lines were transferred to an MSRE medium to regenerate T₀ plants. Tᵢ segregants obtained by self-pollination of marker-free T₀ plants were subjected to further analysis. Table 3 shows the general composition of the culture media for plant tissue used in the Examples.

TABLE 3
The plant tissue culture media used in this study

| Medium | N6D*¹⁾ | N6DNU | N6DSE-H40 | MSRE | ½ MS |
|---|---|---|---|---|---|
| Application | Callus | Agrobacterium | Selection of | Regeneration of | Rooting of T₀ |
| | proliferatio [symbol] | elimination | rice calli | T₀ plants | plants and |
| | | | | | seedling |
| | | | | | germination |

| Basal medium | | N6D | N6D | | N6D | | MS*²⁾ | ½ MS |
|---|---|---|---|---|---|---|---|---|
| Antibiotics | Vancomycin | — | 100 | mg/L | 100 | mg/L | — | — |
| | Meropenem | — | 12.5 | mg/L | 12.5 | mg/L | — | — |
| | Hygromycin | — | — | | 40 | mg/L | — | — |
| Gelling | Gelrite | 4 g/L | 4 | g/L | 4 | g/L | 4 g/L | 4 g/L |
| agent | | | | | | | | |

*¹⁾Toki (1997) Rapid and efficint Agrobacterium-mediated transformation of rice. Plant Mol Biol Rep (15) 16-21 doi: 10.1007/BF02772109
*²⁾Murashige and Skoog (1962) A Revised Medium for Rapid Growth and Bio Assays with Tobacco Cultures. Phisiol Plantar (15) 437-497 doi: 10.1111/j.1399-3054.1962.tb08052.x [symbol]
indicates data missing or illegible when filed

### <PCR and DNA Sequencing Analysis of GT Plants>

A nucleic acid manipulation method comprising preparation of genome DNA and RNA, PCR, CAPS, and DNA sequencing analysis was performed in the same manner as a previously described method (Shimatani Z. et al., Mol Genet Genomics. 281(3):329-344 (2009), Shimatani Z. et al., Nat Biotechnol, 35(5):441-443 (2017)). The primers used in the Examples are shown in Tables 1 and 2.

First, GT candidates having an OsClpP5 KO mutation were screened by junction PCR analysis. A 4.1 kb 5′ junction fragment comprising a promoter region was amplified with Clp 5J-F and pAct-R that are primers (FIG. 4C and Table 1) by using a Tks Gflex DNA polymerase (Takara Bio) according to the instructions of the manufacturer. An equimolar mixture of pOsClpP5KO-Con5 and the “Nipponbare” genome DNA that imitates the state of heterozygosis was used as a control DNA sample. Similarly, a 5.6-kb 3′ junction fragment comprising a region encoding OsClpP5 was detected by PCR analysis using TPase 3-F and Clp 3J-R that are primers (FIG. 4C and Table 1). pOsClpP5KO-Con3 was used as a control. Next, the locus of a target was analyzed by CAPS analysis, and the presence of a restriction enzyme site in OsClpP5 introduced by gene targeting was confirmed. A 920 bp DNA fragment was amplified by PCR using Clp Ex F-6 and Clp Ex-R that are primers and digested using PvuII. The above-described DNA fragment was also used for direct DNA sequencing analysis to confirm a desired KO mutation in OsClpP5 and cloning sequencing analysis using Zero Blunt TOPO PCR cloning kit (Thermo Fisher Scientific) to confirm Ac/Ds-mediated positive marker excision.

Similarly, junction PCR analysis was performed to screen the callus lines of GT candidates having an S549D mutation of OsRacGEF1. Primer pairs of GEF 5J-F/pAct-R and TPase 3-F/GEF 3J-R were used to amplify a 4.2 kb 5′ junction fragment and a 6.1 kb 3′ junction fragment, respectively. A 980 bp DNA fragment amplified by PCR using primers GEF EX F-5 and GEF Ex-R was used for DNA sequencing analysis and CAPS analysis using BssHII.

### Example 1: An Inducible-Type Autonomous Marker Excision System Using Artificially Manipulated Maize Ac/Ds

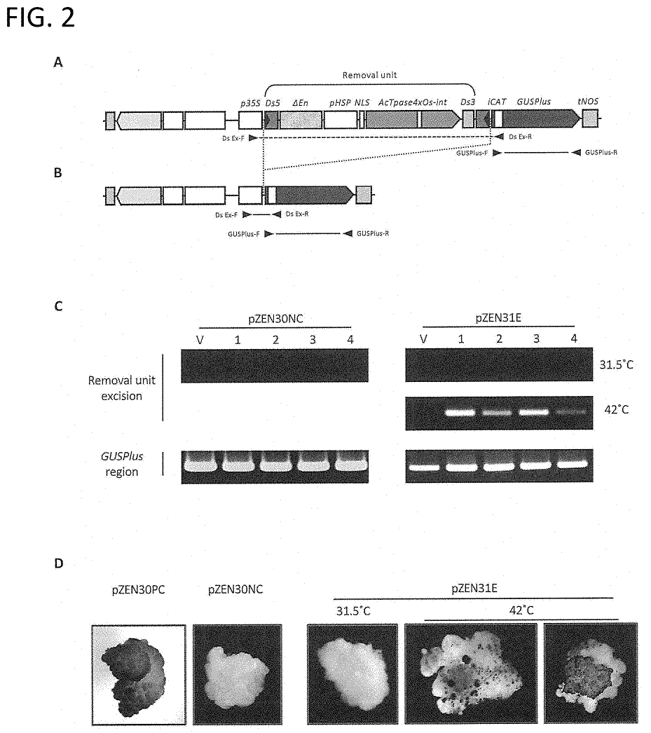

In order to evaluate the function of a heat-shock inducible Ac/Ds-mediated marker excision system, pZEN30NC and pZEN31E, which are plasmid vectors for a GUS reporter assay, were prepared (FIG. 1). These vectors were introduced to rice calli by using transformation mediated by Agrobacterium, thereby making 74 and 196 independent gene recombinant callus lines, respectively (Table 4). Furthermore, about 50 gene recombinant callus lines having pZEN30PC were also made as a control group that constitutively expresses GUSPlus. Subsequently, each callus line having pZEN30NC was analyzed as to whether Ds has been excised. As a result of PCR analysis using primers Ds Ex-F/Ds Ex-R (Table 5), no excision of Ds was detected in all gene recombinant callus lines (Table 4 and FIG. 2C). Similarly, while almost no GUS-positive signal was found in the callus lines introduced with pZEN30NC, almost all of the callus lines introduced with pZEN30PC exhibited a GUS signal (Table 4 and FIG. 2D). This result suggests that a DNA-type transposon system which has a functional compatibility with the Ac/Ds system and is activated by a tissue culture process does not exist in the rice genome.

The callus lines introduced with pZEN31E were divided into two groups. One of the groups was cultured at normal temperature (31.5° C.) as a control while the other group was placed in a heated state at 42° C. for 90 minutes. After nurse culture under normal conditions (31.5° C.) for one week, the genome DNA of each gene recombinant callus line was extracted, and excision of Ds was detected by PCR analysis using primers Ds Ex-F/Ds Ex-R (Table 5). As a result, amplification of a 650 bp fragment exhibiting excision of Ds was observed in 159 (81.1%) and 187 (95.1%) callus lines in the control group and the heat-shock treatment group, respectively (Table 4 and FIG. 2C).

Furthermore, a GUS staining assay was performed to analyze the frequency and temporal and spatial occurrence of Ds excision by AcTPase4xOs under the control of a heat-shock promoter. The result indicates that AcTPase4xOs has a function of removing a synthesized Ds element in a rice callus. Although a Ds element was excised at a low frequency even under the normal conditions, it was revealed that heat-shock treatment increases the frequency of Ds excision mediated by AcTPase4xOs (Table 4 and FIG. 2D).

TABLE 4
Evalualtion of heat-shock inducible Ac/Ds mediated marker excision system

| | | Heat-shock | | | Removal unit | |
|---|---|---|---|---|---|---|
| Vector | Analized | treatment | GUS-positive | (%) | excision | (%) |
| pZEN30NC | 74 | — | 4 | 5.4 | 0 | 0 |
| pZEN31E | 196 | — | 182 | 92.9 | 159 | 81.1 |
| | | 42° C., 90 min | 191 | 97.4 | 187 | 95.1 |

TABLE 5
List of primers for Ac/Ds excision assay

| | | | |
|---|---|---|---|
| No. | Designation | Sequence (5′→3′)^{a} | SEQ ID NO |

Ac/Ds excision assay

| | | | |
|---|---|---|---|
| 1 | Ds Ex-F | AGTCAAGCTTCCGGAAACCTCCTCGGATTCATTGCCCAG | 138 |
| 2 | Ds Ex-R | TGACGAATTCGACGCCATTGAGGTCGAAGACGCCACGGG | 139 |
| 3 | GUSPlus-F | ATGGTAGATCTGAGGGTAAATTTCTAGTTT | 140 |
| 4 | GUSPlus-R | TCACGTGATGGTGATGGTGATGGCTAGC | 141 |
| 5 | hpt GT F-1 | GATATGTCCTGCGGGTAAATAGCTGCGCCG | 142 |
| 6 | t355-R New | GGTACCCTGGATTTTGGTTTTAGGAATTA | 143 |
| 7 | EnSpm No2-R | CGTGCTCTGATCCGGAGGCGGACTAGCATG | 144 |

### Example 2: Design-Based Modification of a Target Gene by Gene Targeting

OsClpP5 is a rice endogenous gene encoding a P5 subunit of an ATP-dependent caseinolytic protease, and disruption of homozygosis causes a chloroplast dysfunction which leads to a leaf color mutation as in pale yellow leaves (Tsugane K. et al., Plant J. 45(1):46-57 (2006)). OsClpP5 was selected as a model to be combined with an autonomous Ac/Ds-mediated marker excision system to technically demonstrate design-based modification by gene targeting.

A gene targeting vector, pOsClpP5KO-AcDs, was designed to be introduced with a mutation in a splice site and a new PvuII site by introducing a nucleotide substitution in the 5′ terminal of intron 1 of OsClpP5 (FIGS. 4A and B). In primary modification, a “removal unit” comprising a transcription terminator (ΔEn), a positive marker (hpt) (the sequence set forth in SEQ ID NO: 44), and a visual marker (EGFP) (the sequence set forth in SEQ ID NO: 49) was inserted to OsClpP5 intron 1 via homologous recombination (FIG. 4C). In the subsequent secondary modification, the removal unit was excised out from a target region by an autonomous Ac/Ds system (FIGS. 5A and B). The obtained transcriptional product of OsClpP5 was expected to comprise intron 1 that has not been subjected to splicing, which results in a premature termination codon. pOsClpP5KO-AcDs was introduced to embryogenic calli derived from a seed of Nipponbare via large-scale Agrobacterium transformation. Callus lines of GT candidates were selected from 67.5 g calli after positive-negative selection for three to four weeks.

In order to screen true GT (TGT) callus lines having OsClpP5 that is properly modified by homologous recombination, primer pairs (Clp 5J-F/pAct-R and TPase 3-F/Clp 3J-R, respectively) were used to perform junction PCR analysis (FIGS. 4C and D). Among 297 GT candidates, 71 callus lines were found as a true gene target. Thus, the frequency of GT in the OsClpP5 locus was estimated to be about 23.9% per live callus (Table 6).

Sequencing analysis of a 5′ junction fragment of each GT callus line revealed that a desired mutation was introduced to the position of a target of OsClpP5 together with a removal unit. Further, fluorescence microscope analysis was performed to confirm 67 GT callus lines (67/71, 94.4%) expressing an EGFP signal (Table 6 and FIG. 4E).

TABLE 6
Summary of GT experiments and target gene modification

| | | | | Plant material for | | | | | | | |
|---|---|---|---|---|---|---|---|---|---|---|---|
| | | Marker | | GT Transformation | | Number of | PCR analysis | | | Sequencing analysis. | |
| Number of | Mutation | excision | Fluorescent | Seeds for | Fresh weight | PN callus | TGT callus | Frequency | EGFP | Desired | |
| experiments | type | system | signal | cellus | of calli (g) | lines | lines | TGT/PN (%) | positive | mutation | WT |

target: OsClpP5 (Os03g0308100) (Cultivar: Nipponbare)

| | | | | | | | | | | | |
|---|---|---|---|---|---|---|---|---|---|---|---|
| 1 | KO | Ac/Ds | EGFP | 250 | 67.5 | 297 | 71 | 23.9 | 67 | ND | ND |

target: OsRacGEF1 (Os09g0544800) (Cultivar: Kinmaze)

| | | | | | | | | | | | |
|---|---|---|---|---|---|---|---|---|---|---|---|
| 3 | S594D | Ac/Ds | EGFP | 1322 | 252.5 | 574 | 59 | 10.3 | ND | ND | ND |
| 1 | S549D | Ds | EGFP | 481 | 92.3 | 113 | 13 | 11.5 | ND | ND | ND |

### Example 3: Heat-Shock Inducible Ac/Ds-Mediated Marker Excision in TGT Callus Lines

The sturdiest 15 TGT callus lines having modified OsClpP5 were used for the following analysis in order to optimize the heat-shock condition for inducing Ac/Ds-mediated marker gene excision.

Each GT callus line was equally divided and treated at 42° C. for 0, 40, 60, or 90 minutes based on the results of the above-described preliminary experiment (FIGS. 2A to D). The treated callus lines were cultured in an N6D medium for four weeks and the frequency of secondary modification was analyzed. It was expected that EGFP expression would function as an indicator of excision of a “removal unit” in a specific cell line. It was considered that when the unit is excised out, expression of EGFP would be removed from corresponding cells and their progeny. Such a cell line, which is a product resulting from the secondary modification, can be distinguished from surrounding EGFP-expressing cells. As expected, all EGFP-positive cells were confirmed to have a “removal unit” by PCR analysis (FIG. 5D). Meanwhile, some EGFP-negative cell lines appeared in 13 or 14 TGT callus lines after treatment with heat-shock for a corresponding exposure time (Table 7 and FIG. 5C). A part of the EGFP-negative calli of each GT line was selected for PCR analysis. As a result, successful removal of a “removal unit” was confirmed in all of the analyzed EGFP-negative calli (Table 7 and FIG. 5D). These results demonstrated the secondary modification of OsClpP5 to remove a positive marker using a heat-shock inducible Ac/Ds system.

The GT callus lines that were treated or not treated with heat shock were transferred to an MSRE medium and cultured for four to six weeks until T₀ plants are regenerated. The regeneration rate of the heat-shock treated group was within the range from 56.3% to 62.5%, while the regeneration rate of the heat-shock untreated control group was 68.8% (Table 7). A sufficient number of T₀ plants were obtained from each group. These results suggest that the heat-shock step is not harmful to plant regeneration.

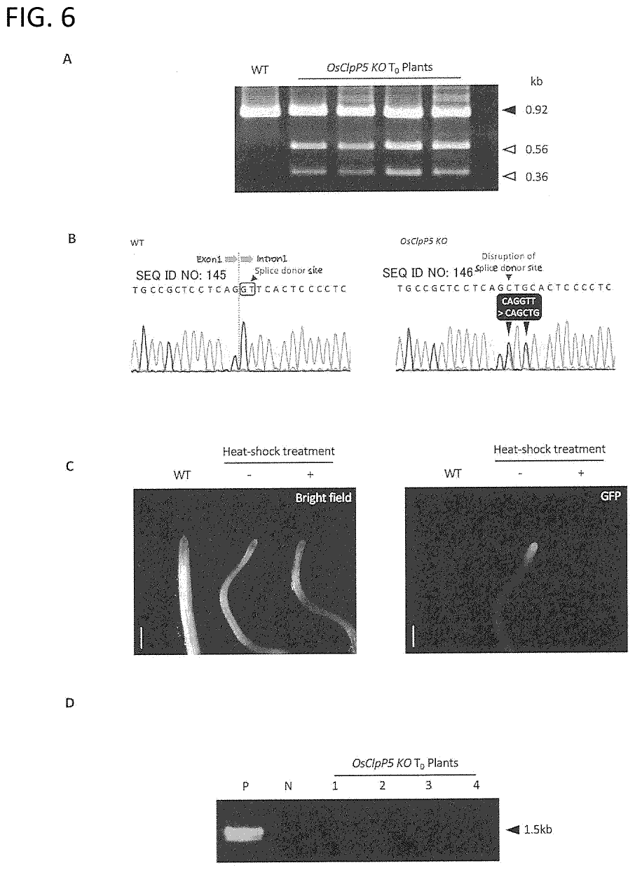

The regenerated T₀ plants were individually proliferated on a ½ MS medium to be subjected to further analysis. CAPS and DNA sequencing analysis indicate that a desired mutation has been introduced to the majority of the T₀ plants derived from the GT callus lines treated with heat shock (FIGS. 6A and B). Furthermore, it was confirmed that almost all of such T₀ plants do not have a “removal unit” by PCR analysis and EGFP expression in root tissue (Table 8, FIGS. 6C and D). Meanwhile, it was confirmed that the frequency of excision of a “removal unit” is clearly low in the T₀ plants derived from the heat-shock untreated group (Table 8).

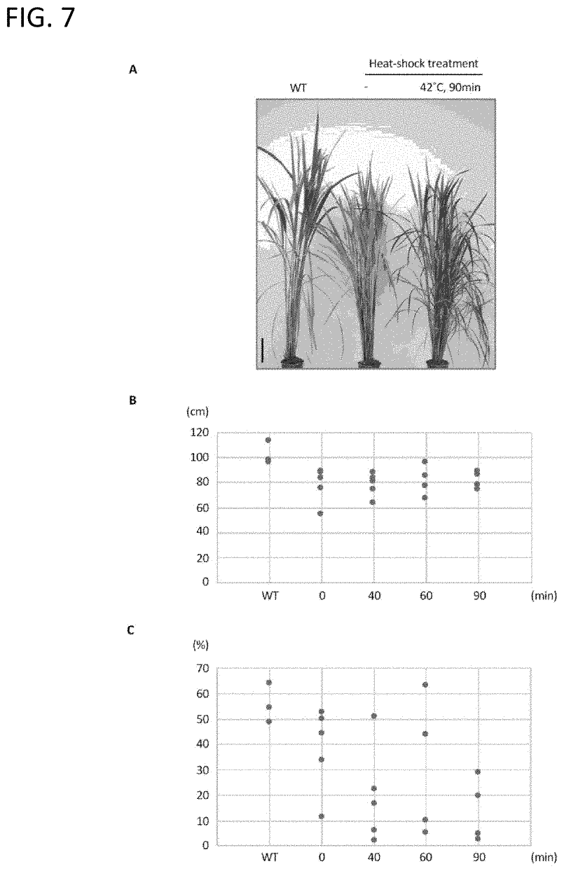

Next, the influence of gene targeting and heat-shock inducible Ac/Ds-mediated marker excision on a rice plant body was studied. In order to evaluate the growth of T₀ plants, the plant height of the plants in the ear emergence period was evaluated. As a result, the plant height of T₀ plants regenerated from marker-free callus lines was confirmed to be relatively low (FIGS. 7A and B). It is considered that this is probably due to a somatic cell mutation that occurred in the gene targeting process, and is barely associated with heat-shock treatment because the plant height of the T₀ plants regenerated from the callus lines was suppressed as compared to WT plants regardless of the presence or absence of the heat-shock treatment (FIG. 7B). Meanwhile, it seems that the plant height of the plants treated with heat shock affects the fertility of the T₀ plants. T₀ plants derived from the GT callus lines after heat-shock treatment at 42° C. for 90 minutes exhibited a relatively low fertility (FIG. 7C). Thus, it was assumed that the heat-shock condition for inducing Ac/Ds-mediated marker excision is 42° C. for 40 minutes that decreases the stress on rice calli as much as possible.

The above-described results demonstrated efficient design-based gene modification in rice calli by gene targeting in combination with an Ac/Ds-mediated autonomous marker excision system. In the next example, progeny of self-pollination of T₀ plants was studied to study the stability and heredity of a desired mutation.

TABLE 7
Heat-shock inducible Ac/Ds - mediated marker excision
from TGT callus lines carrying modified OsClpP5

| Treatment | | Marker gene excision | | | | |
|---|---|---|---|---|---|---|
| time | Analyzed | EGFP-negative | PCR-negative | Frequency (%) | Regenerated | Frequency (%) |
| 0 | 16 | 0 | 0 | 0.0 | 11 | 68.8 |
| 40 | | 13 | 13 | 81.3 | 9 | 56.3 |
| 60 | | 13 | 13 | 81.3 | 10 | 62.5 |
| 90 | | 14 | 14 | 87.5 | 10 | 62.5 |

TABLE 8
Genotyping of Tₒ plants carrying modified OsClpP5

| Treatment time | TGT | | KO mutation | Marker excised | |
|---|---|---|---|---|---|
| (min) | callus lines | Analyzed | CAPS-positive | Marker excised | Frequency (%) |
| 0 | 7 | 73 | 63 | 14 | 28.6 |
| 40 | 5 | 69 | 66 | 66 | 100 |
| 60 | 5 | 39 | 37 | 33 | 89.2 |
| 90 | 5 | 53 | 45 | 44 | 97.8 |

### Example 4: Analysis of Marker-Free T₁ Progeny Having an OsClpP5 KO Mutation

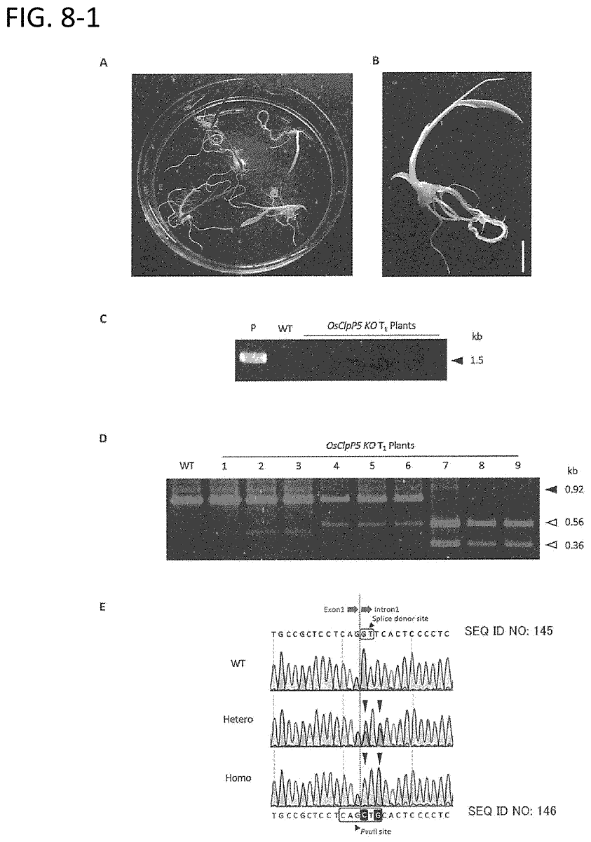

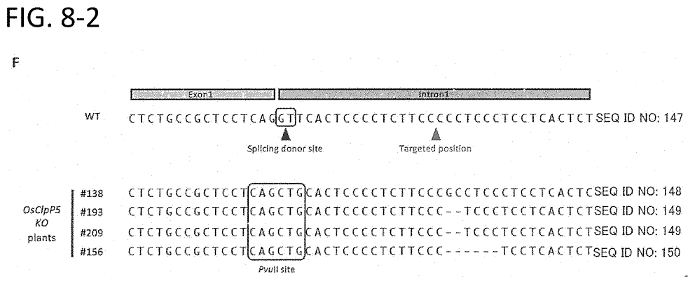

Marker-free plants of the T₀ generation obtained by two-stage mutation has an OsClpP5 KO mutation as a heterozygous recessive mutation. Thus, for the phenotype of their T₁ progeny, it was expected that the normal phenotype and the albino phenotype would be segregated at a ratio of 3:1. ¼ of the T₁ progeny derived from each T₀ plant actually exhibited the albino phenotype (Table 9, FIGS. 8A and B). Furthermore, the result of genotyping by CAPS and DNA sequencing analysis was consistent with the phenotype of each T₁ plant (FIGS. 8D and E). These results demonstrated the accuracy and efficiency of modification of a target gene through gene targeting and Ac/Ds-mediated marker excision.

Table 9
Segregation of modified OsClpP5 in T₁ generation

| | | Normal | | Albino |
|---|---|---|---|---|
| Line | Analyzed | WT | Hetero | null |
| 156-2 | 18 | 5 | 7 | 6 |
| 193-19 | 18 | 3 | 10 | 5 |
| 209-6 | 19 | 7 | 7 | 4 |
| 156-5 | 20 | 8 | 8 | 4 |
| 156-1 | 19 | 7 | 10 | 2 |

### Example 5: A Footprint Sequence after Ac/Ds-Mediated Marker Excision

The genome sequence of albino plants segregated in the T₁ generation was analyzed to confirm whether a target locus was properly edited after two-stage modification by gene targeting after Ac/Ds-mediated marker excision. A footprint sequence generated in association with Ac/Ds excision was observed as 1 bp insertion or 2 to 6 bp deletion (FIG. 8F). It was revealed that the plants derived from the same GT callus line share the same footprint sequence. This result means that a removal unit was excised at the very early stage after heat-shock treatment, and the T₀ plants were regenerated from proliferated calli in the same cell line.

### Example 6: Modification of OsRacGEF1

There are many receptor kinases to recognize pathogenic infection on a cell membrane of a plant. OsRacGEF1 was identified as an important component of “defensome” of rice. OsRacGEF1 is critically involved in the early stage of a chitin-driven immune response and plays an important role in a signaling pathway. In rice, chitin elicitor receptor kinase 1 (OsCERK1) and a chitin elicitor-binding protein (OsCEBiP) form a receptor complex to recognize and activate a downstream signaling pathway. OsRacGEF1 functions as a relay point of the signaling pathway. OsRacGEF1 directly interacts with OsCERK1 and is activated by phosphorylation of S549 at the C terminal thereof, thereby activating OsRac1 which is a main control factor of rice immunity. OsRacGEF1 S549D, which is a mutation that imitates the phosphorylation, was found to increase the resistance to Magnaporthe oryzae via activation of OsRac1 (Akamatsu A. et al., Cell Host Microbe, 13(4):465-476 (2013)).

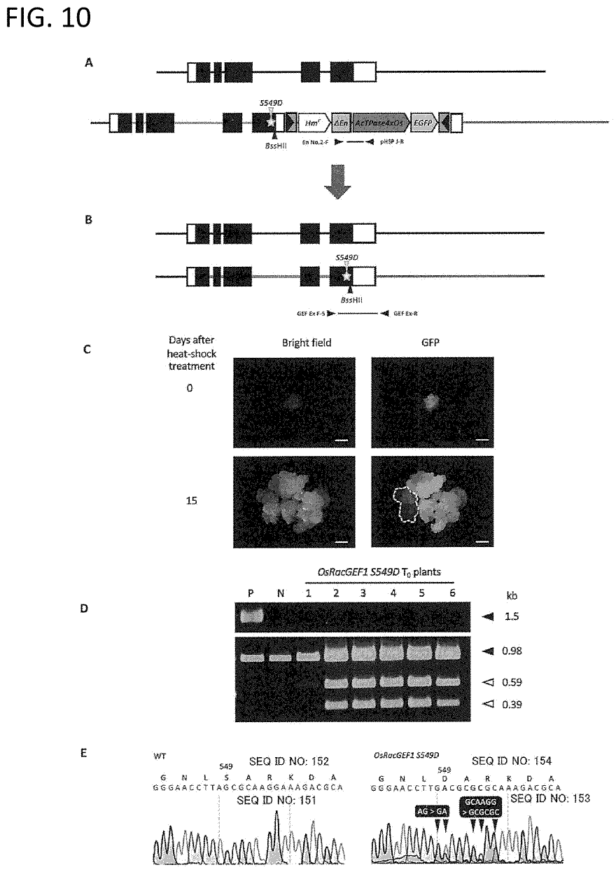

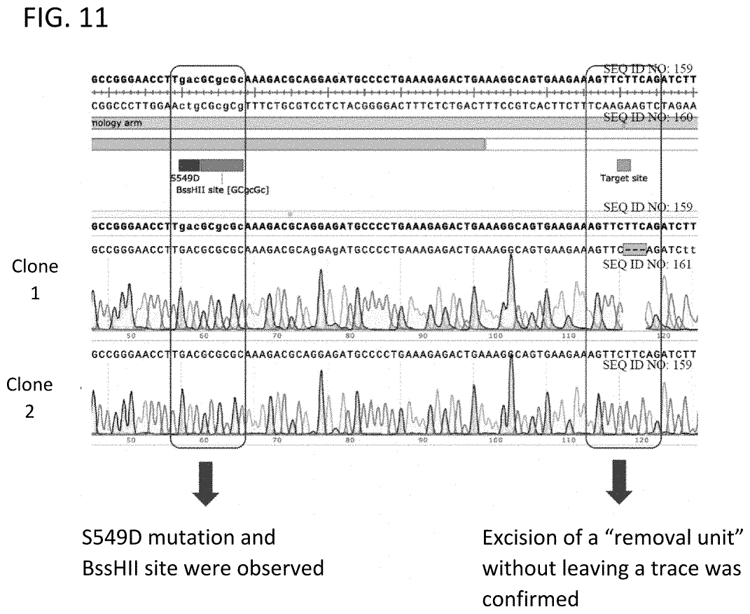

A gene targeting vector of OsRacGEF1 was designed so as to introduce a nucleotide substitution causing a silent mutation that causes an S549D mutation and a new BssHII site (FIGS. 9A and B). In primary modification, a “removal unit” comprising a transcription terminator (ΔEn) and a visual marker (EGFP) was inserted to the 3′UTR region of OsRacGEF1 via homologous recombination (FIG. 9B). In the subsequent secondary modification, the removal unit is excised out from the target region by an autonomous Ac/Ds system. It was confirmed that obtained OsRacGEF1 has an S549D mutation (FIGS. 10A and B, and FIG. 11). Furthermore, deletion of the “removal unit” without leaving a trace was confirmed (FIG. 11).

Large-scale Agrobacterium transformation and positive-negative selection were performed to introduce pGEF1S549D-AcDs to embryogenic calli derived from a seed of “Kinmaze”. After repeating for three times, 1322 GT candidate callus lines were obtained from 252.5 g calli. In order to screen true GT (TGT) callus lines having OsRacGEF1 which is properly modified by HR, primer pairs (GEF 5-J-F/pAct-R and TPase 3-F/GEF 3J-R, respectively) were used to perform 5′- and 3′-junction PCR analysis (FIG. 9C and Table 2). As a result, 59 callus lines were found as a true gene target. Thus, the GT frequency in the OsRacGEF1 locus was estimated to be about 10.3% per live callus (Table 6).

As described above, pGEF1S549D-Ds was also introduced to Kinmaze. 13 callus lines among 113 candidates were confirmed to be TGT by 5′- and 3′-junction PCR analysis. The frequency of GT was 11.5% relative to the positive-negative-selected callus lines (Table 6). A desired mutation and EGFP expression were also observed.

Excision of an Ac/Ds-mediated maker from the modified OsRacGEF1 locus was subsequently performed as described above. 16 TGT callus lines having a desired mutation were equally divided and treated at 42° C. for 40, 60, or 90 minutes. After nurse culture for four weeks, EGFP-negative cell lines were obtained (FIG. 10C). These calli were selected for regeneration of T₀ plants. The regeneration frequency was 37.5 to 62.5% (Table 10). As a result of PCR and CAPS analysis of T₀ plants, successful removal of a removal unit and introduction of a desired mutation to a targeted position in OsRacGEF1 were confirmed (Table 11, FIGS. 10D and E). These T₀ plants were grown to confirm a desired mutation heredity segregation in the T₁ generation through self-pollination.

TABLE 10
Heat-shock inducible Ac/Ds -mediated marker excision
from TGT callus lines carrying modified OsRacGEF1

| Treatment | | Marker gene excision | | | | |
|---|---|---|---|---|---|---|
| time | Analyzed | EGFP-negative | PCR-negative | Frequency (%) | Regenerated | Frequency (%) |
| 0 | 15 | 0 | ND | ND | 3 | 20.0 |
| 40 | | 15 | ND | ND | 10 | 66.7 |
| 60 | | 15 | ND | ND | 10 | 66.7 |
| 90 | | 15 | ND | ND | 6 | 40.0 |

TABLE 11
Genotyping of T₀ plants carrying modified OsRacGEF1

| Treatment time | TGT | | KO mutation | | | Marker excised | |
|---|---|---|---|---|---|---|---|
| (min) | callus lines | Analyzed | CAPS-positive | KO mutation | Frequency (%) | Marker excised | Frequency (%) |
| 0 | 3 | 157 | ND | ND | | 2 | 1.3 |
| 40 | 7 | 56 | 13 | ND | | 13 | 23.2 |
| 60 | 5 | 41 | 20 | ND | | 18 | 43.9 |
| 90 | 3 | 57 | 1 | ND | | 1 | 1.8 |

### Example 7: Analysis of a Transcriptional Product in an OsClpP5 KO Variant

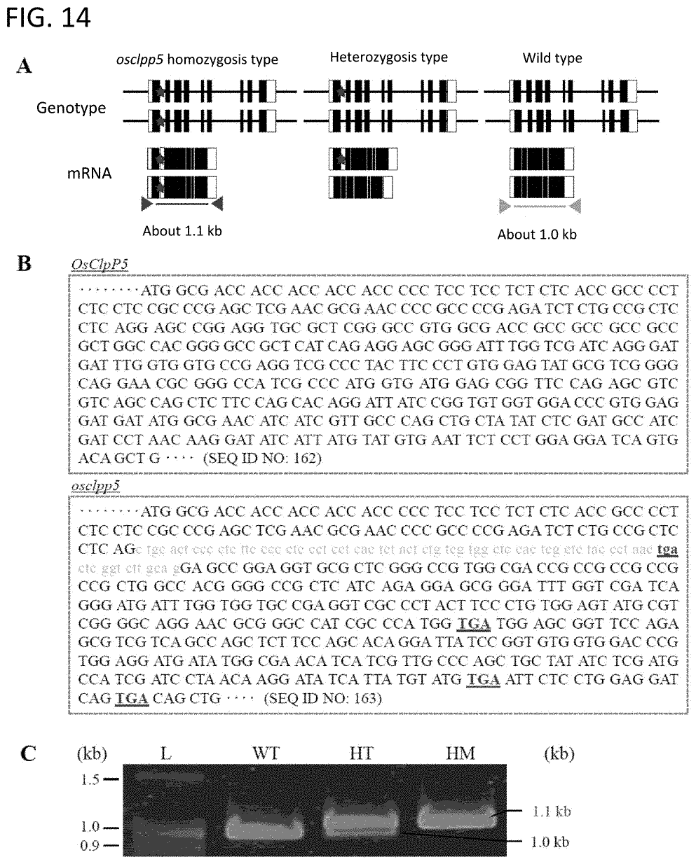

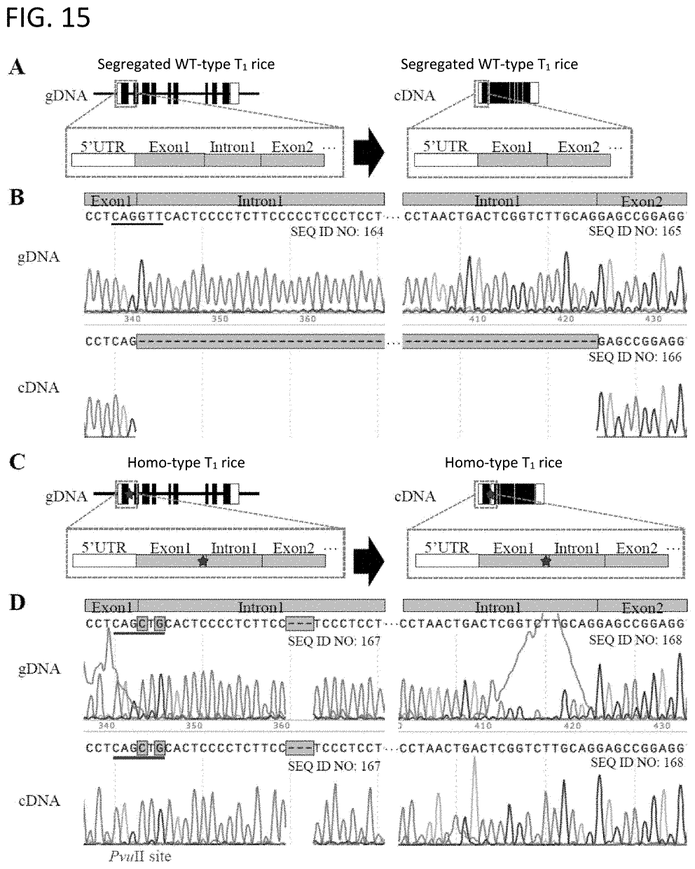

OsClpP5 modification in this example was designed to cause gene function disruption by splicing inhibition via base substitution. Specifically, a splicing donor site positioned on the 5′ side terminal of the OsClpP5 first intron was modified to thereby modify GT to CT. It was expected that an immature termination codon would consequently appear by a frame shift mutation or the like due to the first intron sequence in the mRNA derived from the OsClpP5 gene, and a normal protein would not be translated, which would lead to disruption of the function. Thus, in order to confirm the accurate edition of a target gene and the function modification thereby, T₁ plant bodies were developed for two lines that had experienced successful OsClpP5 modification by the method of Example 2 and classified into a wild type, a heterozygosis type, and an osclpp5 homozygosis type which is an albino mutant based on the phenotype and the genotype of each individual, and the transcriptional products thereof were analyzed (FIGS. 14A to C and FIGS. 15A to D). First, an mRNA was isolated from each plant body, and a cDNA was obtained by a reverse transcription reaction using a primer set (Clp 5UTR-F No1; ACCACCCTCTCCCGGATAAGAGGCGCAACC (SEQ ID NO: 157) and Clp 3UTR-R No1; TGTGGAATCGCAAAACTATCTTGCCAAGCT (SEQ ID NO: 158)) set in the 5′ UTR and 3′ UTR regions of the OsClpP5 gene. Since a cDNA derived from the wild-type OsClpP5 is about 1.0 kb but a cDNA in the albino mutant comprises a 83 bp sequence corresponding to the first intron, the cDNA should be detected as an about 1.1 kb DNA fragment (FIGS. 14A, B, and C). As a result of fractionation of the OsClpP5 cDNA of each plant body by agarose gel electrophoresis, an about 1.0 kb DNA fragment and an about 1.1 kb DNA fragment were detected from the wild-type plant body and the osclpp5 homozygosis-type plant body, respectively as expected, while both DNA fragments were observed in the heterozygosis-type plant body (FIG. 14C). Cloning sequence analysis was subsequently performed for the obtained cDNAs to observe a splicing out of the first intron in the mRNA derived from OsClpP5 in the wild-type individual (FIGS. 15A and B). Meanwhile, it was confirmed that about 80 bases derived from the first intron remain in the mRNA derived from the osclpp5 homozygosis-type plant bodies (FIGS. 15C and D). Further, appearance of an immature termination codon due to the first intron sequence was observed.

The above results demonstrated the modification of a splicing pattern of a target gene by gene targeting and the function disruption thereby.

### INDUSTRIAL APPLICABILITY

The present invention enables minute and quick target gene modification as a plant variety improving technique. Unlike the conventional genome editing techniques, the present invention enables modification of a wide range (to a few kb), and also enables Cis genesis, knock-in modification and the like, wherein an artificial sequence such as a marker gene is autonomously removed after establishment of modification of a target gene. Furthermore, it is also possible to perform “pyramiding” for accumulating useful genes in a short period of time. Accordingly, the present invention can be expected to be applied as a practical breeding technique in variety improvement of higher plants.

The present application is on the basis of Japanese Patent Application No. 2019-030971 filed in Japan (filing date: Feb. 22, 2019), whose entire content is encompassed herein.

1. A nucleic acid comprising:
(1) a transposon; and
(2) a sequence homologous to at least a part of a target region of a double-stranded DNA of a plant cell,
wherein the transposon comprises a marker gene and a gene encoding a transposase operably linked to an inducible promoter.

2. The nucleic acid of claim 1, wherein the transposon is derived from a transposon selected from the group consisting of a Ds element, an Ac element, an Spm-s element, an En1 element, an Spm-I8 (dSpm) element, an Mu element, a Tam1 element, a Tam2 element, a Tam3 element, an nDart element, a Dart element, and a PiggyBac element.

3. The nucleic acid of claim 1, wherein the transposon is derived from a Ds element or an Ac element, and the transposase is AcTPase or a variant thereof.

4. The nucleic acid of claim 1, wherein the marker gene includes a gene encoding a fluorescent protein.

5. The nucleic acid of claim 1, wherein the inducible promoter is a heat-shock inducible promoter.

6. A method for producing plant cells that have a mutation in a target region of a double-stranded DNA and do not have any of an exogenous transposon and a gene encoding a transposase, the method comprising:
(1) the step of selecting plant cells introduced with the nucleic acid of claim 1;
(2) the step of culturing the plant cells selected in step (1) under a condition wherein the inducible promoter is activated; and
(3) the step of selecting a cell in which expression of the marker protein is eliminated from a population comprising the plant cells cultured in step (2).

7. The method of claim 6, wherein the plant cells are cells of a monocotyledon.

8. The method of claim 7, wherein the monocotyledon is a gramineous plant.

9. A plant cell obtained by the method of claim 6 or a plant body comprising the cell.

10. The nucleic acid of claim 2, wherein the transposon is derived from a Ds element or an Ac element, and the transposase is AcTPase or a variant thereof.

11. The nucleic acid of claim 10, wherein the marker gene includes a gene encoding a fluorescent protein.

12. The nucleic acid of claim 11, wherein the inducible promoter is a heat-shock inducible promoter.


SEQUENCE LISTING (168 sequences)

SEQ ID NO: 1 - DNA, 11 bp, Artificial Sequence
  terminal inverted repeat for AcTPase
cagggatgaaa

SEQ ID NO: 2 - DNA, 11 bp, Artificial Sequence
  terminal inverted repeat for AcTPase
tagggatgaaa

SEQ ID NO: 3 - DNA, 11 bp, Artificial Sequence
  terminal inverted repeat for AcTPase
gagggatgaaa

SEQ ID NO: 4 - DNA, 11 bp, Artificial Sequence
  terminal inverted repeat for AcTPase
tagagatgaaa

SEQ ID NO: 5 - DNA, 11 bp, Artificial Sequence
  terminal inverted repeat for AcTPase
gagctatgaaa

SEQ ID NO: 6 - DNA, 11 bp, Artificial Sequence
  terminal inverted repeat for AcTPase
tttcatcccta

SEQ ID NO: 7 - DNA, 11 bp, Artificial Sequence
  terminal inverted repeat for AcTPase
tttcatctgag

SEQ ID NO: 8 - DNA, 11 bp, Artificial Sequence
  terminal inverted repeat for AcTPase
tttcatcccta

SEQ ID NO: 9 - DNA, 11 bp, Artificial Sequence
  terminal inverted repeat for AcTPase
tttcatccctg

SEQ ID NO: 10 - DNA, 11 bp, Artificial Sequence
  terminal inverted repeat for AcTPase
tttcatctcta

SEQ ID NO: 11 - DNA, 13 bp, Artificial Sequence
  terminal inverted repeat for EnSpm/CACTA
cactacaagaaaa

SEQ ID NO: 12 - DNA, 13 bp, Artificial Sequence
  terminal inverted repeat for EnSpm/CACTA
cactacaacaaaa

SEQ ID NO: 13 - DNA, 13 bp, Artificial Sequence
  terminal inverted repeat for EnSpm/CACTA
cactacaaaaaaa

SEQ ID NO: 14 - DNA, 13 bp, Artificial Sequence
  terminal inverted repeat for EnSpm/CACTA
cactataagaaaa

SEQ ID NO: 15 - DNA, 13 bp, Artificial Sequence
  terminal inverted repeat for EnSpm/CACTA
cactacgccaaaa

SEQ ID NO: 16 - DNA, 13 bp, Artificial Sequence
  terminal inverted repeat for EnSpm/CACTA
cactaccggaatt

SEQ ID NO: 17 - DNA, 13 bp, Artificial Sequence
  terminal inverted repeat for EnSpm/CACTA
ttttcttgtagtg

SEQ ID NO: 18 - DNA, 13 bp, Artificial Sequence
  terminal inverted repeat for EnSpm/CACTA
ttttgttgtagtg

SEQ ID NO: 19 - DNA, 13 bp, Artificial Sequence
  terminal inverted repeat for EnSpm/CACTA
tttttttgtagtg

SEQ ID NO: 20 - DNA, 13 bp, Artificial Sequence
  terminal inverted repeat for EnSpm/CACTA
ttttcttatagtg

SEQ ID NO: 21 - DNA, 13 bp, Artificial Sequence
  terminal inverted repeat for EnSpm/CACTA
ttttggcgtagtg

SEQ ID NO: 22 - DNA, 13 bp, Artificial Sequence
  terminal inverted repeat for EnSpm/CACTA
aattccggtagtg

SEQ ID NO: 23 - DNA, 13 bp, Artificial Sequence
  terminal inverted repeat for PiggyBac
ccctagaaagata

SEQ ID NO: 24 - DNA, 35 bp, Artificial Sequence
  terminal inverted repeat for PiggyBac
ccctagaaagatagtctgcgtaaaattgacgcatg

SEQ ID NO: 25 - DNA, 35 bp, Artificial Sequence
  terminal inverted repeat for PiggyBac
catgcgtcaattttacgcagactatctttctaggg

SEQ ID NO: 26 - DNA, 13 bp, Artificial Sequence
  terminal inverted repeat for PiggyBac
tatctttctaggg

SEQ ID NO: 27 - DNA, 35 bp, Artificial Sequence; identical to SEQ ID NO: 25
  terminal inverted repeat for PiggyBac

SEQ ID NO: 28 - DNA, 35 bp, Artificial Sequence; identical to SEQ ID NO: 24
  terminal inverted repeat for PiggyBac

SEQ ID NO: 29 - DNA, 2118 bp, Artificial Sequence
  AcTPase 4x gene optimized for rice
  CDS (1)..(2118)
atggccatcgtgcatgagccacagccacaaccgcaaccacagccagagcctcagcctcaacctcagcctgagccagaggaggaggccccacaaaagcgcgctaagaagtgcacctccgacgtctggcagcacttcaccaagaaggagatcgaggtcgaggttgacggcaagaagtacgtccaggtctggggccactgcaacttcccaaactgcaaggccaagtacagggccgagggccatcatggcacatccggctttaggaaccacctcaggacctcccactctctcgtgaagggccaactctgcctcaagtccgagaaggaccacggcaaggacatcaacctcatcgagccgtacaagtacgacgaggtggtgtccctcaagaagctccacctcgccatcattatgcacgagtacccgttcaacatcgtcgagcacgcctacttcgtcgagttcgtgaagtccctcaggccgcacttcccgatcaagtctagagtgaccgcccgcaagtacatcatggacctctaccttgaggagaaggagaagctctacggcaagctcaaggacgtgcagtccaggttctccaccacgatggacatgtggaccagctgccagaacaagtcctacatgtgcgtgaccatccactggatcgacgacgactggtgcctccagaagaggatcgtcggcttcttccatgtggccggcagacatacaggccagaggctctcccaaaccttcaccgcgatcatggtcaagtggaacatcgagaagaagctgttcgccctctccctcgataacgcctccgctaatgaggtggccgtgcacgacatcatcgaggacctccaggacaccgactccaaccttgtgtgcgacggcgccttctttcatgttcgctgcgcctgccacatcctcaacctggttgctaaggatggcctcgccgtgatcgccggcaccattgagaagatcaaggccatcgtcctcgccgtcaagtcctctccacttcagtgggaggagctgatgaagtgcgcgtccgagtgcgatctcgacaagtccaagggcatcagctacgccgtgtccaccaggtggaatagcacctacctcatgctccgcgacgccctctactacaagccagccctcatcaggctcaagacctccgatccaagacgctacgatgccatctgcccaaaggccgaggagtggaagatggcgctcaccctcttcaagtgcctgaagaagttcttcgacctcaccgagctgctctccggcacccaatactctaccgccaacctcttctacaagggcttctgcgagatcaaggacctgatcgcccaatggtgcgtgcacgagaagttcgtgattcgcagaatggccgtggccatgagcgagaagtttgagaagtactggaaggtgtccaatatcgccctcgcggtggcctgcttcctcgatccaaggtacaagaagatcctgatcgagttctacatgaagaagtttcacggcgactcctacaaggtgcacgtcgacgatttcgtgcgcgtgatccgcaagctctaccagttctactcctcctgctctccatccgccccaaagaccaagaccaccaccaacgacagcatggacgacaccctcatggagaacgaggacgacgagttccagaactacctccacgagctgaaggactacgaccaggtcgagagcaacgagctggacaagtacatgtccgagccgctcttgaagcactccggccagttcgatatcctctcatggtggcgcggcagagtggccgagtacccaatcctcacacagatcgccagagatgtgctcgccatccaggtgtcaacagtggcttctgagtctgccttttccgctggcggcagggtggtggatccatacagaaatagactcggctccgagatcgtcgaggccctcatctgcactaaggactgggtggcagcctctaggaagggcgccacatacttcccgaccatgattggcgaccttgaggtgctcgacagcgttatcgccgctgccaccaaccacgagaaccacatggacgaggatgaggacgccatcgagttcagcaagaacaatgaggacgtggccagcggctcctccccatga

SEQ ID NO: 30 - protein, 705 aa, Artificial Sequence
  Synthetic Construct
MAIVHEPQPQPQPQPEPQPQPQPEPEEEAPQKRAKKCTSDVWQHFTKKEIEVEVDGKKYVQVWGHCNFPNCKAKYRAEGHHGTSGFRNHLRTSHSLVKGQLCLKSEKDHGKDINLIEPYKYDEVVSLKKLHLAIIMHEYPFNIVEHAYFVEFVKSLRPHFPIKSRVTARKYIMDLYLEEKEKLYGKLKDVQSRFSTTMDMWTSCQNKSYMCVTIHWIDDDWCLQKRIVGFFHVAGRHTGQRLSQTFTAIMVKWNIEKKLFALSLDNASANEVAVHDIIEDLQDTDSNLVCDGAFFHVRCACHILNLVAKDGLAVIAGTIEKIKAIVLAVKSSPLQWEELMKCASECDLDKSKGISYAVSTRWNSTYLMLRDALYYKPALIRLKTSDPRRYDAICPKAEEWKMALTLFKCLKKFFDLTELLSGTQYSTANLFYKGFCEIKDLIAQWCVHEKFVIRRMAVAMSEKFEKYWKVSNIALAVACFLDPRYKKILIEFYMKKFHGDSYKVHVDDFVRVIRKLYQFYSSCSPSAPKTKTTTNDSMDDTLMENEDDEFQNYLHELKDYDQVESNELDKYMSEPLLKHSGQFDILSWWRGRVAEYPILTQIARDVLAIQVSTVASESAFSAGGRVVDPYRNRLGSEIVEALICTKDWVAASRKGATYFPTMIGDLEVLDSVIAAATNHENHMDEDEDAIEFSKNNEDVASGSSP

SEQ ID NO: 31 - DNA, 25 bp, Artificial Sequence
  right border sequences of T-DNA
tggcaggatatataccgttgtaatt

SEQ ID NO: 32 - DNA, 25 bp, Artificial Sequence
  left border sequences of T-DNA
cggcaggatatattcaattgtaaat

SEQ ID NO: 33 - DNA, 21 bp, Artificial Sequence
  NLS-coding sequence derived from SV40
ccaaagaagaagagaaaggtc

SEQ ID NO: 34 - DNA, 300 bp, Artificial Sequence
  subterminal region (Ds5)
cagggatgaaagtaggatgggaaaatcccgtaccgaccgttatcgtataaccgattttgttagttttatcccgatcgatttcgaacccgaggtaaaaaacgaaaacggaacggaaacgggatatacaaaacggtaaacggaaacggaaacggtagagctagtttcccgaccgtttcaccgggatcccgtttttaatcgggatgatcccgtttcgttaccgtattttctaattcgggatgactgcaatatggccagctccaactcccatccataaccactgaggcccagcccatgtaagaa

SEQ ID NO: 35 - DNA, 300 bp, Artificial Sequence
  subterminal region (Ds3)
tgctccagatttatatggattttatctatgtttaattaagacttgtgtttacaattttttatatttgtttttaagttttgaatatatgttttcatgtgtgattttaccgaacaaaaataccggttcccgtccgatttcgactttaacccgaccggatcgtatcggttttcgattaccgtatttatcccgttcgttttcgttaccggtatatcccgttttcgtttccgtcccgcaagttaaatatgaaaatgaaaacggtagaggtattttaccgaccgttaccgaccgttttcatcccta

SEQ ID NO: 36 - DNA, 29839 bp, Artificial Sequence
  Vector sequence of pClpP5 AcDsEG-D
  promoter (7)..(536): p35S (CaMV 35S promoter)
  gene (553)..(1284): DT-A gene with intron1 of Caster bean Cat1 gene
  terminator (1338)..(1536): t35S (CaMV 35S terminator)
  misc_feature (1593)..(4592): OsClpP5 5′ side homologous region for HR
  misc_feature (4593)..(4892): 5′ terminal region of Ds element (Ds5)
  promoter (4916)..(5753): pAct1 (promoter region of rice Act1 gene)
  5′UTR (5754)..(6327): 5′UTR region of rice Act1 gene with intron
  gene (6328)..(7353): hpt (Hygromycin phosphotransferase gene)
  terminator (7375)..(7574): t35S (CaMV 35S terminator)
  terminator (7635)..(8611): delta En/Spm
  promoter (8626)..(9394): pHSP (Heat shock promoter)
  gene (9411)..(11626): AcTPase4x gene with intron and SV40 NLS
  terminator (11665)..(11917): tNOS (NOS terminator)
  promoter (11943)..(12472): p35S (CaMV 35S promoter)
  gene (12489)..(13200): EGFP gene
  terminator (13228)..(13526): tE9 (pea rbcS E9 terminator)
  misc_feature (13615)..(13914): 3′ terminal region of Ds element (Ds3)
  misc_feature (13915)..(16912): OsClpP5 3′ side homologous region for HR
  terminator (16962)..(17155): t35S (CaMV 35S terminator) Complementary strand
  gene (17163)..(17750): DT-A gene Complementary strand
  promoter (17751)..(19747): pUbi (Maize Ubiquitin promoter)
  misc_signal (20298)..(20322): T-DNA RB
  promoter (21040)..(21144): AmpR promoter
  gene (21145)..(22006): AmpR gene
  rep_origin (22177)..(22765): Ori
  rep_origin (23435)..(23629): pVS1 oriV
  gene (23695)..(24768): pVS1 RepA
  gene (25197)..(25826): pVS1 StaA
  gene (27730)..(28521): SmR
  misc_signal (29079)..(29103): T-DNA LB
gaattcccatggagtcaaagattcaaatagaggacctaacagaactcgccgtaaagactggcgaacagttcatacagagtctcttacgactcaatgacaagaagaaaatcttcgtcaacatggtggagcacgacacgcttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacagggtacccggggatctatggatcccgacgatgtaaatttctagtttttctccttcattttcttggttaggacccttttctctttttatttttttgagctttgatctttctttaaactgatctattttttaattgattggttatggtgtaaatattacatagctttaactgataatctgattactttatttcgtgtgtctatgatgatgatgatagttacaggttgtcgactcttctaaatcttttgtgatggaaaacttttcttcgtaccacgggactaaacctggttatgtagattccattcaaaaaggtatacaaaagccaaaatctggtacacaaggaaattatgacgatgattggaaagggttttatagtaccgacaataaatacgacgctgcgggatactctgtagataatgaaaacccgctctctggaaaagctggaggcgtggtcaaagtgacgtatccaggactgacgaaggttctcgcactaaaagtggataatgccgaaactattaagaaagagttaggtttaagtctcactgaaccgttgatggagcaagtcggaacggaagagtttatcaaaaggttcggtgatggtgcttcgcgtgtagtgctcagccttcccttcgctgaggggagttctagcgttgaatatattaataactgggaacaggcgaaagcgttaagcgtagaacttgagattaattttgaaacccgtggaaaacgtggccaagatgcgatgtatgagtatatggctcaagcctgtgcaggaaatcgtgtcaggcgatcttagcatgcccgctgaaatcaccagtctctctctacaaatctatctctctctataataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccaggggtacctcgagtaactataacggtcctaaggtagcgattaattaaatttaaatcgtcgacgtgactgtttagtgaaaataactcatgaaatattttgcagatggatccttatctaagaattggtgaagacttgcaactctatgtaaggttgcaatctgatctaggtaactatggttctgatagcgatcaagaaatcgctagatctgtactttctgactgcaggacaaaagtggggattaatgatcagcgagtacttgatgtagttgcttgtgcactgtgtaatttaactgaggtaaaggttgattttgccttctatttgctgttgacactgcacgttgtgttcttattgaggtcacatgctgttgacagcaatggttgagtgcagttatctaatattcttggaattgctattgccagatggacaaggatgtactggtgaaggagctcacagaaatgtttacacctgaagaggtgcccttgtttgggtcaaattcagcatttgactgggccaattttcatgttcaggcattttctgatgaatccctttcttttgatgaggtaggtacattaaaactggtgtctttaatgattcatctggtggaagctcttataaaattctttgatggtgcaatgatccttgtaactgtaccttaccattatgaaaattattgttactttttgtatcttctcagttgtcttctgcattgtaggagtgttcaagaacctcttcagtagatggtgggttgcatgagtcaccaattacaaacaccggtagctcgatatcaaagactacgatgccacaatctgttcctcgtgtcttaggtgttggccagttgcttgaatcggtgagtacttatcagctgcctttgcacatggtttgctagttgctgctactcagctagctctagttgccctaatcttattaattgttaaggtggcatggaaccataacatgttgtgtttgtacccctttctgctgctgtcaaaatagtatttatcacgtggtggattcggctacaaatatattatggagcatttgaatctatgtaacattttctctactgtgaactcatatctttgtaatcaggcgttacatgtagccggccaagttgcgggagcatctgtttcgacctccccgctcccatacggcacaatgaccagtcaatgcgaagccttgggatcaggcactaggaagaagctctcaagctggcttgtcaatggccatgactctacgccagacaatcctgctccaagccttccatctgcgcagcatttcatcattcctaaagtacatgatttgtcctgtctttctcccatgtcattctggaatgttgcctattctgcatctgtgtgtatcatctggccctcacgatcgcaatcgacattcatccaggtaaattcatgcggtttcgagagcagcatccggacgactttggagccttgctcggcagtgaagctcccacccgccagccccttcgacaacttcctgaaggctgcatatcgtgcccagtagcagccatgacaagccttcgagattatgcctaaccatgtttagtagtgctaggatgtagtacctgagcatcagaactagattatcttttgaaagacttggccgatctggcttatcctttattatctgtagcactgttagttgtagtagcttgtgtatcaatgtatggctatgtctgtgtccctccagtaaatgacaatcgtatatgtactatgtagtatgaccatgtagcccactagtaaatgtttgtagggtatgtatgacaatgtatgaccatattgtttttttgagatgtaggattctatttcagactcgagtaatctgtgggggacatgttcgtcgacctccggtgctatttttccttttatcactgcaaggattagatggtttcctgcaatatttgcatgctattcgcgcggatgaaattgtgaagtcatgggtttctcaagtccacaatcattagttcacattattacggcgttcatgaatattacattttggaggatcttatttctatagtttttctatatagctctttgaatcaagaaatgtttttcatgtgatctaatcaaaaggtcatttctatgtttgcaattatctattttacatttacatttctatcaaaatcttttttttgttttttttcctatttatctgttttttttattcctgcgattcaaagtgacgcttaaaatttagggaaaattgcaaaaaccatcctataaatcacttgaaacttgacattccactttataagccattttattgcaaataccacccatctactctgtctaatcgaatcaacatgttttcatgaattatgagctgtcaatgaagcttttaccaaaattaaatatttatatagtacagtcttgacacttttgtattgagtttcgctgtattttaaaaaaaattaattaaacatgaagaataaatctgatacaaaagcacttggacaagattgactcgactagttttaacctgtggtatccctaccccagcgataatgggttgattcggttaaacacgttcgcactagaatggtttttacaaaaaaaaagaactgagtaggggtggacatttacaataaaataacttatgagatgtaatgtgaaatttcaagtgatttatagggttgtttttttttaaattccctagaatttaacgggtgtgtgtctggtggacgtccgtggtgcgcgcgcgcgcgcgagggaatgatcaaaaaccttttcggggcgtcacgtaagcccatgacccgacgtccaaaggcctaccaacaccgtcggcatggcccgcgtcttctcctcgtcttcctctctctcgagtctcgaagaagacacacactattctctcttcttcttaccaccctctcccggataagaggcgcaaccaaagcccccaccctcgcccaaaacccccacgagccgcggccatggcgaccaccaccaccaccccctcctcctctctcaccgcccctctcctccgcccgagctcgaacgcgaaccccgccccgagatctctgccgctcctcagctgcactcccctcttccgcagggatgaaagtaggatgggaaaatcccgtaccgaccgttatcgtataaccgattttgttagttttatcccgatcgatttcgaacccgaggtaaaaaacgaaaacggaacggaaacgggatatacaaaacggtaaacggaaacggaaacggtagagctagtttcccgaccgtttcaccgggatcccgtttttaatcgggatgatcccgtttcgttaccgtattttctaattcgggatgactgcaatatggccagctccaactcccatccataaccactgaggcccagcccatgtaagaagcggccgcactagcatactcgaggtcattcatatgcttgagaagagagtcgggatagtccaaaataaaacaaaggtaagattacctggtcaaaagtgaaaacatcagttaaaaggtggtataaagtaaaatatcggtaataaaaggtggcccaaagtgaaatttactcttttctactattataaaaattgaggatgtttttgtcggtactttgatacgtcatttttgtatgaattggtttttaagtttattcgcttttggaaatgcatatctgtatttgagtcgggttttaagttcgtttgcttttgtaaatacagagggatttgtataagaaatatctttaaaaaaacccatatgctaatttgacataatttttgagaaaaatatatattcaggcgaattctcacaatgaacaataataagattaaaatagctttcccccgttgcagcgcatgggtattttttctagtaaaaataaaagataaacttagactcaaaacatttacaaaaacaacccctaaagttcctaaagcccaaagtgctatccacgatccatagcaagcccagcccaacccaacccaacccaacccaccccagtccagccaactggacaatagtctccacacccccccactatcaccgtgagttgtccgcacgcaccgcacgtctcgcagccaaaaaaaaaaaaagaaagaaaaaaaagaaaaagaaaaaacagcaggtgggtccgggtcgtgggggccggaaacgcgaggaggatcgcgagccagcgacgaggccggccctccctccgcttccaaagaaacgccccccatcgccactatatacatacccccccctctcctcccatccccccaaccctaccaccaccaccaccaccacctccacctcctcccccctcgctgccggacgacgagctcctcccccctccccctccgccgccgccgcgccggtaaccaccccgcccctctcctctttctttctccgttttttttttccgtctcggtctcgatctttggccttggtagtttgggtgggcgagaggcggcttcgtgcgcgcccagatcggtgcgcgggaggggcgggatctcgcggctggggctctcgccggcgtggatccggcccggatctcgcggggaatggggctctcggatgtagatctgcgatccgccgttgttgggggagatgatggggggtttaaaatttccgccatgctaaacaagatcaggaagaggggaaaagggcactatggtttatatttttatatatttctgctgcttcgtcaggcttagatgtgctagatctttctttcttctttttgtgggtagaatttgaatccctcagcattgttcatcggtagtttttcttttcatgatttgtgacaaatgcagcctcgtgcggagcttttttgtaggtagaccggggggcaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccggccgtctggaccgatggctgtgtagaagtactcgccgatagtggaaaccgacgccccagcactcgtccgggatcctctagagtcgacctgcaggcatgcccgctgaaatcaccagtctctctctacaaatctatctctctctataataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccaggggtacccctagtctctcttaaggtagcatcacaagtttgtacaaaaaagcaggcttccctcgagggcaacttctgtccaagaccaattgatgccattgggtgtgataggagggcaaatgatgccgtgggcacctcgccagccaggcatttggccaccgatgcaaacacagatgccaccgccgatgccgtggggatttcctcctcgtgggcagtcacaatcaccaggattgccctcacactcaccaggatcagtacgttaagttgatatcctttgcatctctatttgcttcgttgtttaagcagttactagaaaacatgcatgtatatgttgcagtctatgtatatgtttaattagttactcggtaaactaacaaatgtttgtttcttttaaagggttcaggctcacatcatgctagtccgcctccggatcagagcacgtttatggacttattgatgaacacaagtggcggcggctccaatgacccaccaacagaatgaattaatatggaggcttgtgtggaacttactatgattgcgttttgtatggactttaacttgttttagatggatttgaacttctttcgtatggacttgaacttgtatgaatattgaatatggtgcttgtgttatgttatgttgaatatggtgcttgtgttgtgatatattgaatgttgtgcttatattgtgctgttatggaggcttcccatccggggagggagaaaaataaaattggatattaaaaaaaattattcactaagagtgtcggcccccacactcttatatgcgcccaggtagcttactgatgtgcgcgcagtaagagtgacggccacggtactggccgacacttttaacataagagtgtcggttgcttgttgaaccgacacttttaacataagagcgtcggtccccacacttctatacgaataagagcgtccattttagagtgacggctaagagtgtcggtcaaccgacactcttatacttagagtgtcggcttatttcagtaagagtgtggggttttggccgaagctgggcccgctagcgtttaaatgctagcccagtgaaagcagtgaattgaagcattcccgaaacccactggaatgatctagtactcactctacgatgtacagtgaagtaatacttcaaaactggtgtaatttggtatgccaaaaggactccatagtttcacgacatatttccaaacggttcaggatcagtactgcccatctgcctggggcccacactagcgggcaattggttctcgtagtttctcgttctcaatcaatcattccatactcgctatcccctccatcacagaataaatgcaacaatgagtttccgtgtacaaatttaatcgttcgtcttatttaaaatattttttaaaaaactaaaaaacaaaagtcacgcataaagtactattcatgttttataatctaataacagtataaatactaatcataaaaaaaaattcaaataagatggacgattaaagttgaacactgaaattcatggctgcttttgttttgagactgagggagtacacgataagatttgatcgcaatcaaagtaacctacatcaaagaagcaagatatgtgggggaaaaatgaatactctagagcaaattaaggtgagccccgctttgtagaggctgatggagtactggagcgacggaagcgaagcagatcgagtgtgctgtaaagcgaaacgagcaagaaccagagaagtccagagatttcaggacagattagttgtgaacctataaatatcctgcctcattccccaacctccatccatcgagccaagactgaagcatttgatcgagctcctgcagaaccttggccatggctccaaagaagaagagaaaggtcatggccatcgtgcatgagccacagccacaaccgcaaccacagccagagcctcagcctcaacctcagcctgagccagaggaggaggccccacaaaagcgcgctaagaagtgcacctccgacgtctggcagcacttcaccaagaaggagatcgaggtcgaggttgacggcaagaagtacgtccaggtctggggccactgcaacttcccaaactgcaaggccaagtacagggccgagggccatcatggcacatccggctttaggaaccacctcaggacctcccactctctcgtgaagggccaactctgcctcaagtccgagaaggaccacggcaaggacatcaacctcatcgagccgtacaagtacgacgaggtggtgtccctcaagaagctccacctcgccatcattatgcacgagtacccgttcaacatcgtcgagcacgcctacttcgtcgagttcgtgaagtccctcaggccgcacttcccgatcaagtctagagtgaccgcccgcaagtacatcatggacctctaccttgaggagaaggagaagctctacggcaagctcaaggacgtgcagtccaggttctccaccacgatggacatgtggaccagctgccagaacaagtcctacatgtgcgtgaccatccactggatcgacgacgactggtgcctccagaagaggatcgtcggcttcttccatgtggccggcagacatacaggccagaggctctcccaaaccttcaccgcgatcatggtcaagtggaacatcgagaagaagctgttcgccctctccctcgataacgcctccgctaatgaggtggccgtgcacgacatcatcgaggacctccaggacaccgactccaaccttgtgtgcgacggcgccttctttcatgttcgctgcgcctgccacatcctcaacctggttgctaaggatggcctcgccgtgatcgccggcaccattgagaagatcaaggccatcgtcctcgccgtcaagtcctctccacttcagtgggaggagctgatgaagtgcgcgtccgagtgcgatctcgacaagtccaagggcatcagctacgccgtgtccaccaggtggaatagcacctacctcatgctccgcgacgccctctactacaagccagccctcatcaggctcaagacctccgatccaagacggtatgtttgtctcaattgttgtacatgtcatcattataaattctcaattaatcaaatgtcaattattgtagctacgatgccatctgcccaaaggccgaggagtggaagatggcgctcaccctcttcaagtgcctgaagaagttcttcgacctcaccgagctgctctccggcacccaatactctaccgccaacctcttctacaagggcttctgcgagatcaaggacctgatcgcccaatggtgcgtgcacgagaagttcgtgattcgcagaatggccgtggccatgagcgagaagtttgagaagtactggaaggtgtccaatatcgccctcgcggtggcctgcttcctcgatccaaggtacaagaagatcctgatcgagttctacatgaagaagtttcacggcgactcctacaaggtgcacgtcgacgatttcgtgcgcgtgatccgcaagctctaccagttctactcctcctgctctccatccgccccaaagaccaagaccaccaccaacgacagcatggacgacaccctcatggagaacgaggacgacgagttccagaactacctccacgagctgaaggactacgaccaggtcgagagcaacgagctggacaagtacatgtccgagccgctcttgaagcactccggccagttcgatatcctctcatggtggcgcggcagagtggccgagtacccaatcctcacacagatcgccagagatgtgctcgccatccaggtgtcaacagtggcttctgagtctgccttttccgctggcggcagggtggtggatccatacagaaatagactcggctccgagatcgtcgaggccctcatctgcactaaggactgggtggcagcctctaggaagggcgccacatacttcccgaccatgattggcgaccttgaggtgctcgacagcgttatcgccgctgccaccaaccacgagaaccacatggacgaggatgaggacgccatcgagttcagcaagaacaatgaggacgtggccagcggctcctccccatgaattggtgaccagctcggagatctcacgtgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggggcggccgcactagtcctgcaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacagggtacctagggatctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatctagagttaacctagcttgttcgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacactagatccccaaacggtaccgtttaaacactagtgtcaaagggcgaattcgacccagctttcttgtacaaagtggtgatgttaacctcgaggtgctccagatttatatggattttatctatgtttaattaagacttgtgtttacaattttttatatttgtttttaagttttgaatatatgttttcatgtgtgattttaccgaacaaaaataccggttcccgtccgatttcgactttaacccgaccggatcgtatcggttttcgattaccgtatttatcccgttcgttttcgttaccggtatatcccgttttcgtttccgtcccgcaagttaaatatgaaaatgaaaacggtagaggtattttaccgaccgttaccgaccgttttcatccctagctccctcctcacttactctgtcgtggctccactcgctctaccctaactgactcggtcttgcaggagccggaggtgcgctcgggccgtggcgaccgccgccgccgccgctggccacggggccgctcatcagaggagcgggatttggtcgatcaggtgggggggggggttgattttgtgacgaaccatttgtagctctgcatcgtctggttgggggagggttgggtggggatgcgagggagctgatgatgctgtggttgttgtgtgtttcgtagggatgatttggtggtgccgaggtcgccctacttccctgtggagtatgcgtcggggcaggaacgcgggccatcgcccatggtgatggagcggttccagagcgtcgtcagccagctcttccagcacgtacgggagcactagattgttgcacaagtttatttgtggagggttctgtgtgctgtctgactctgtgtgattgtggaacagaggattatccggtgtggtggacccgtggaggatgatatggcgaacatcatcgttgcccagctgctatatctcgatgccatcgatcctaacaaggtgaaaatgatgaataagacgctatccgacatccaattcctatttcctataccaatagctgccaaagctttgttacgaactgcttactctaaaaatactgaggcgatgaagggagtggtgctcactgtatttacaattttacatgaactatatggtgtcttgttttaatggatgattgttgttaatccccagataaaatggtccttttatctgtcaatactcaataccagacactaaagtaccattgctactcactttgggttatatatgagtccttgttagtcctagcttaggctgtgattcagttatgtagttgtgacctcaaaccttatggtgaggcttgtattgcgatttcatctaccaggacctgattccatggcaccatatattcatgatttcgtacttacattcagtactcgattcttgacttcctctcaacattcctgtaggatatcattatgtatgtgaattctcctggaggatcagtgacagctggtaataactcatccattttttttcatagctccatctcactcttggaacatttgtttcacaccaaactgcattttgtgtttagggatggccatattcgacacgatgaagcatatcagacctgatgtttccacagtttgtattggacttgctgcaaggtatttagtactgaaaaactgatatccttaattggcaattgatttggatttgtgacgctctgcaatttatgcatcttataatgtttaattctttagcattctgttcaattattcaataagggtttcagttgctatattctggtcaacatgtaaacatgcacctgtttccagttgtatatcccgcctaattccttctcctttcataatgtgggaagcacaccaaatcattgattgtatcaggcatgttctattcagtataatcagcctcaatctaggattttgggttgactgagatcaaattttgtgctgcgcatgttagcaaagaagctttgtaccctcatacctccttaatcaaaatataatttttcaaactttatgtttgtgagaatccatatggatgttgataacaggagagtttcttcatttcatgtactttatcaatgtttagccaacaatgcaaattcaggagtgcttatcagtattccaggcacacagtttattagtaaaacatgataactgttcagctgcaaatctgtaaccatttctttctcattgtacttttggaatgctctcttttagctatccatcttgccttaagttgcattgatttcggttatgcactcattttcactgttttgtttccctttttggcagtatgggagcttttctgcttagtgctgggacaaaaggtaattgagtgattgttaatgcaagtactacatttattctacaactagactgatctgaatccttgaatactgtacttgtgaatgtaacacagggaagcgatacagcttacctaactcaagaataatgatccatcaacctctcggaggagcccaaggacaagagactgatcttgagatccaggtaaatccacaaagttaaattgtgtttttatcaagatcaggacgacatctcaatagcaaataattaaccatatcattgtttctctcttgccggtatatggttcgatctacatgaaatacatttatttccacaagcgaatagcaactccgctggctttctattgttacttgtatcaccagaagatttttctgtcactgattttccattatttcttttaggctaatgagatgctgcatcacaaggctaacctgaatggatacctagcataccacactgggcagcccctagataagatcaacgtagatactgaccgtgattacttcatgagcgcgaaggaggcaaaggagtatggtctaattgatggagttatcatgaatccccttaaagcccttcaaccgcttcctgcttctagttagccatggagtgctcaatctccacggagcattttttggttatcttttagaactgttattgcatccactgtttttattagcttggcaagatagttttgcgattccacaagcaaccacatcctgaggcttcaaagtttgtacaatacagatgtactactaggaggatatcttctgcgatgaatattgcaacttatttgatgtactattaggaggatatcttctgcgatgaatattgcaacttatttgatactccctctgtttcatgccaagtcgatttgattttttttccttagacaattttctgtaggtttgactaaatttatcaaaaaattagcataatctacaacactaaattagttctattaaaataacattgaatatattttgataatatgtttgttgtattaaaatgttgctatattttttatatacttgatcaaattttaaaaaagattatctataaaaaaattaaaacgatttataatatgatgaaaggcgcgcctagggataacagggtaatctcgagaattcgagctcggtaccgctgaaatcaccagtctctctctacaaatctatctctctctataataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagggggcatgctaagatcgcctgacacgatttcctgcacaggcttgagccatatactcatacatcgcatcttggccacgttttccacgggtttcaaaattaatctcaagttctacgcttaacgctttcgcctgttcccagttattaatatattcaacgctagaactcccctcagcgaagggaaggctgagcactacacgcgaagcaccatcaccgaaccttttgataaactcttccgttccgacttgctccatcaacggttcagtgagacttaaacctaactctttcttaatagtttcggcattatccacttttagtgcgagaaccttcgtcagtcctggatacgtcactttgaccacgcctccagcttttccagagagcgggttttcattatctacagagtatcccgcagcgtcgtatttattgtcggtactataaaaccctttccaatcatcgtcataatttccttgtgtaccagattttggcttttgtatacctttttgaatggaatctacataaccaggtttagtcccgtggtacgaagaaaagttttccatcacaaaagatttagaagaatcaacaacatcatcaggatccatctgcagaagtaacaccaaacaacagggtgagcatcgacaaaagaaacagtaccaagcaaataaatagcgtatgaaggcagggctaaaaaaatccacatatagctgctgcatatgccatcatccaagtatatcaagatcaaaataattataaaacatacttgtttattataatagataggtactcaaggttagagcatatgaatagatgctgcatatgccatcatgtatatgcatcagtaaaacccacatcaacatgtatacctatcctagatcgatatttccatccatcttaaactcgtaactatgaagatgtatgacacacacatacagttccaaaattaataaatacaccaggtagtttgaaacagtattctactccgatctagaacgaatgaacgaccgcccaaccacaccacatcatcacaaccaagcgaacaaaaagcatctctgtatatgcatcagtaaaacccgcatcaacatgtatacctatcctagatcgatatttccatccatcatcttcaattcgtaactatgaatatgtatggcacacacatacagatccaaaattaataaatccaccaggtagtttgaaacagaattaattctactccgatctagaacgaccgcccaaccagaccacatcatcacaaccaagacaaaaaaaagcatgaaaagatgacccgacaaacaagtgcacggcatatattgaaataaaggaaaagggcaaaccaaaccctatgcaacgaaacaaaaaaaatcatgaaatcgatcccgtctgcggaacggctagagccatcccaggattccccaaagagaaacactggcaagttagcaatcagaacgtgtctgacgtacaggtcgcatccgtgtacgaacgctagcagcacggatctaacacaaacacggatctaacacaaacatgaacagaagtagaactaccgggccctaaccatggaccggaacgccgatctagagaaggtagagagggggggggggggaggacgagcggcgtaccttgaagcggaggtgccgacgggtggatttgggggagatctggttgtgtgtgtgtgcgctccgaacaacacgaggttggggaaagagggtgtggagggggtgtctatttattacggcgggcgaggaagggaaagcgaaggagcggtgggaaaggaatcccccgtagctgccggtgccgtgagaggaggaggaggccgcctgccgtgccggctcacgtctgccgctccgccacgcaatttctggatgccgacagcggagcaagtccaacggtggagcggaactctccagaggggtccagaggcagcgacagagatgccgtgccgtctgcttcgcttggcccgacgcgacgctgctggttcgctggttggtgtccgttagactcgtcgacggcgtttaacaggctggcattatctactcgaaacaagaaaaatgtttccttagtttttttaatttcttaaagggtatttgtttaatttttagtcactttattttattctattttatatctaaattattaaataaaaaaactaaaatagagttttagttttcttaatttagaggctaaaatagaataaaatagatgtactaaaaaaattagtctataaaaaccattaaccctaaaccctaaatggatgtactaataaaatggatgaagtattatataggtgaagctatttgcaaaaaaaaaggagaacacatgcacactaaaaagataaaactgtagagtcctgttgtcaaaatactcaattgtcctttagaccatgtctaactgttcatttatatgattctctaaaacactgatattattgtagtactatagattatattattcgtagagtaaagtttaaatatatgtataaagatagataaactgcacttcaaacaagtgtgacaaaaaaaatatgtggtaattttttataacttagacatgcaatgctcattatctctagagaggggcacgaccgggtcacgctgcactgcagtctagagggggtaccgtcgacaagctgggatcccagctgggatcccagcttggctgcaggtcgacggatcccccgatgagctaagctagctatatcatcaatttatgtattacacataatatcgcactcagtctttcatctacggcaatgtaccagctgatataatcagttattgaaatatttctgaatttaaacttgcatcaataaatttatgtttttgcttggactataatacctgacttgttattttatcaataaatatttaaactatatttctttcaagatgggaattaacatctacaaattgccttttcttatcgaccatgtacgtaagcgcttacgtttttggtggacccttgaggaaactggtagctgttgtgggcctgtggtctcaagatggatcattaatttccaccttcacctacgatggggggcatcgcaccggtgagtaatattgtacggctaagagcgaatttggcctgtagacctcaattgcgagctttctaatttcaaactattcgggcctaacttttggtgtgatgatgctgactggcaggatatataccgttgtaatttgagctcgtgtgaataagtcgctgtgtatgtttgtttgattgtttctgttggagtgcagcccatttcaccggacaagtcggctagattgatttagccctgatgaactgccgaggggaagccatcttgagcgcggaatgggaatggatttcgttgtacaacgagacgacagaacacccacgggaccgagcttcgaattattccggatgagcattcatcaggcgggcaagaatgtgaataaaggccggataaaacttgtgcttatttttctttacggtctttaaaaaggccgtaatatccaggaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacacacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcgatcctgctcgcgcaggctgggtgccaagctctcgggtaacatcaaggcccgatccttggagcccttgccctcccgcacgatgatcgtgccgtgatcgaaatccagatccttgacccgcagttgcaaaccctcactgatccgcatgcccgttccatacagaagctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctaacaattcgttcaagccgacgccgcttcgcggcgcggcttaactcaagcgttagatgcactaagcacataattgctcacagccaaactatcaggtcaagtctgcttttattatttttaagcgtgcataataagccctacacaaattgggagatatatcatgaaaggctggctttttcttgttatcgcaatagttggcgaagtaatcgcaacatccgcattaaaatctagcgagggctttactaagctagcttgcttggtcgttccggtaccgtgaacgtcggctcgattgtacctgcgttcaaatactttgcgatcgtgttgcgcgcctgcccggtgcgtcggctgatctcacggatcgactgcttctctcgcaacgccatccgacggatgatgtttaaaagtcccatgtggatcactccgttgccccgtcgctcaccgtgttggggggaaggtgcacatggctcagttctcaatggaaattatctgcctaaccggctcagttctgcgtagaaaccaacatgcaagctccaccgggtgcaaagcggcagcggcggcaggatatattcaattgtaaatggcttcatgtccgggaaatctacatggatcagcaatgagtatgatggtcaatatggagaaaaagaaagagtaattaccaattttttttcaattcaaaaatgtagatgtccgcagcgttattataaaatgaaagtacattttgataaaacgacaaattacgatccgtcgtatttataggcgaaagcaataaacaaattattctaattcggaaatctttatttcgacgtgtctacattcacgtccaaatgggggcttagatgagaaacttcacgatcgatgccttgatttcgccattcccagatacccatttcatcttcagattggtctgagattatgcgaaaatatacactcatatacataaatactgacagtttgagctaccaattcagtgtagcccattaccttacataattcactcaaatgctaggcagtctgtcaactcggcgtcaatttgtcggccactatacgatagttgcgcaaattttcaaagtcctggcctaacatcacacctctgtcggcggcgggtcccatttgtgataaatccaccatcacaatagatagtctaatggacgaaaaaggcgaatatttcgatgctgagattcgacgcaattaattcgagaaaaatcccgtgattgatgctgttgagttaccaataatatgggcagcgaaggccatttaattataagatctggtaccaagctgggatcccagcttgcatgcccatcgatgggtcgac

SEQ ID NO: 37 - DNA, 530 bp, Cauliflower mosaic virus
ccatggagtcaaagattcaaatagaggacctaacagaactcgccgtaaagactggcgaacagttcatacagagtctcttacgactcaatgacaagaagaaaatcttcgtcaacatggtggagcacgacacgcttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggaca

SEQ ID NO: 38 - DNA, 530 bp, Cauliflower mosaic virus
ccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggaca

SEQ ID NO: 39 - DNA, 732 bp, Artificial Sequence
  DT-A gene with intron1 of Caster bean Cat1 gene
atggatcccgacgatgtaaatttctagtttttctccttcattttcttggttaggacccttttctctttttatttttttgagctttgatctttctttaaactgatctattttttaattgattggttatggtgtaaatattacatagctttaactgataatctgattactttatttcgtgtgtctatgatgatgatgatagttacaggttgtcgactcttctaaatcttttgtgatggaaaacttttcttcgtaccacgggactaaacctggttatgtagattccattcaaaaaggtatacaaaagccaaaatctggtacacaaggaaattatgacgatgattggaaagggttttatagtaccgacaataaatacgacgctgcgggatactctgtagataatgaaaacccgctctctggaaaagctggaggcgtggtcaaagtgacgtatccaggactgacgaaggttctcgcactaaaagtggataatgccgaaactattaagaaagagttaggtttaagtctcactgaaccgttgatggagcaagtcggaacggaagagtttatcaaaaggttcggtgatggtgcttcgcgtgtagtgctcagccttcccttcgctgaggggagttctagcgttgaatatattaataactgggaacaggcgaaagcgttaagcgtagaacttgagattaattttgaaacccgtggaaaacgtggccaagatgcgatgtatga

SEQ ID NO: 40 - DNA, 199 bp, Cauliflower mosaic virus
gctgaaatcaccagtctctctctacaaatctatctctctctataataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccaggggtac

SEQ ID NO: 41 - DNA, 200 bp, Cauliflower mosaic virus
gctgaaatcaccagtctctctctacaaatctatctctctctataataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccaggggtacc

SEQ ID NO: 42 - DNA, 3000 bp, Oryza sativa
tgactgtttagtgaaaataactcatgaaatattttgcagatggatccttatctaagaattggtgaagacttgcaactctatgtaaggttgcaatctgatctaggtaactatggttctgatagcgatcaagaaatcgctagatctgtactttctgactgcaggacaaaagtggggattaatgatcagcgagtacttgatgtagttgcttgtgcactgtgtaatttaactgaggtaaaggttgattttgccttctatttgctgttgacactgcacgttgtgttcttattgaggtcacatgctgttgacagcaatggttgagtgcagttatctaatattcttggaattgctattgccagatggacaaggatgtactggtgaaggagctcacagaaatgtttacacctgaagaggtgcccttgtttgggtcaaattcagcatttgactgggccaattttcatgttcaggcattttctgatgaatccctttcttttgatgaggtaggtacattaaaactggtgtctttaatgattcatctggtggaagctcttataaaattctttgatggtgcaatgatccttgtaactgtaccttaccattatgaaaattattgttactttttgtatcttctcagttgtcttctgcattgtaggagtgttcaagaacctcttcagtagatggtgggttgcatgagtcaccaattacaaacaccggtagctcgatatcaaagactacgatgccacaatctgttcctcgtgtcttaggtgttggccagttgcttgaatcggtgagtacttatcagctgcctttgcacatggtttgctagttgctgctactcagctagctctagttgccctaatcttattaattgttaaggtggcatggaaccataacatgttgtgtttgtacccctttctgctgctgtcaaaatagtatttatcacgtggtggattcggctacaaatatattatggagcatttgaatctatgtaacattttctctactgtgaactcatatctttgtaatcaggcgttacatgtagccggccaagttgcgggagcatctgtttcgacctccccgctcccatacggcacaatgaccagtcaatgcgaagccttgggatcaggcactaggaagaagctctcaagctggcttgtcaatggccatgactctacgccagacaatcctgctccaagccttccatctgcgcagcatttcatcattcctaaagtacatgatttgtcctgtctttctcccatgtcattctggaatgttgcctattctgcatctgtgtgtatcatctggccctcacgatcgcaatcgacattcatccaggtaaattcatgcggtttcgagagcagcatccggacgactttggagccttgctcggcagtgaagctcccacccgccagccccttcgacaacttcctgaaggctgcatatcgtgcccagtagcagccatgacaagccttcgagattatgcctaaccatgtttagtagtgctaggatgtagtacctgagcatcagaactagattatcttttgaaagacttggccgatctggcttatcctttattatctgtagcactgttagttgtagtagcttgtgtatcaatgtatggctatgtctgtgtccctccagtaaatgacaatcgtatatgtactatgtagtatgaccatgtagcccactagtaaatgtttgtagggtatgtatgacaatgtatgaccatattgtttttttgagatgtaggattctatttcagactcgagtaatctgtgggggacatgttcgtcgacctccggtgctatttttccttttatcactgcaaggattagatggtttcctgcaatatttgcatgctattcgcgcggatgaaattgtgaagtcatgggtttctcaagtccacaatcattagttcacattattacggcgttcatgaatattacattttggaggatcttatttctatagtttttctatatagctctttgaatcaagaaatgtttttcatgtgatctaatcaaaaggtcatttctatgtttgcaattatctattttacatttacatttctatcaaaatcttttttttgttttttttcctatttatctgttttttttattcctgcgattcaaagtgacgcttaaaatttagggaaaattgcaaaaaccatcctataaatcacttgaaacttgacattccactttataagccattttattgcaaataccacccatctactctgtctaatcgaatcaacatgttttcatgaattatgagctgtcaatgaagcttttaccaaaattaaatatttatatagtacagtcttgacacttttgtattgagtttcgctgtattttaaaaaaaattaattaaacatgaagaataaatctgatacaaaagcacttggacaagattgactcgactagttttaacctgtggtatccctaccccagcgataatgggttgattcggttaaacacgttcgcactagaatggtttttacaaaaaaaaagaactgagtaggggtggacatttacaataaaataacttatgagatgtaatgtgaaatttcaagtgatttatagggttgtttttttttaaattccctagaatttaacgggtgtgtgtctggtggacgtccgtggtgcgcgcgcgcgcgcgagggaatgatcaaaaaccttttcggggcgtcacgtaagcccatgacccgacgtccaaaggcctaccaacaccgtcggcatggcccgcgtcttctcctcgtcttcctctctctcgagtctcgaagaagacacacactattctctcttcttcttaccaccctctcccggataagaggcgcaaccaaagcccccaccctcgcccaaaacccccacgagccgcggccatggcgaccaccaccaccaccccctcctcctctctcaccgcccctctcctccgcccgagctcgaacgcgaaccccgccccgagatctctgccgctcctcagctgcactcccctcttccg

SEQ ID NO: 43 - DNA, 838 bp, Oryza sativa
gtcattcatatgcttgagaagagagtcgggatagtccaaaataaaacaaaggtaagattacctggtcaaaagtgaaaacatcagttaaaaggtggtataaagtaaaatatcggtaataaaaggtggcccaaagtgaaatttactcttttctactattataaaaattgaggatgtttttgtcggtactttgatacgtcatttttgtatgaattggtttttaagtttattcgcttttggaaatgcatatctgtatttgagtcgggttttaagttcgtttgcttttgtaaatacagagggatttgtataagaaatatctttaaaaaaacccatatgctaatttgacataatttttgagaaaaatatatattcaggcgaattctcacaatgaacaataataagattaaaatagctttcccccgttgcagcgcatgggtattttttctagtaaaaataaaagataaacttagactcaaaacatttacaaaaacaacccctaaagttcctaaagcccaaagtgctatccacgatccatagcaagcccagcccaacccaacccaacccaacccaccccagtccagccaactggacaatagtctccacacccccccactatcaccgtgagttgtccgcacgcaccgcacgtctcgcagccaaaaaaaaaaaaagaaagaaaaaaaagaaaaagaaaaaacagcaggtgggtccgggtcgtgggggccggaaacgcgaggaggatcgcgagccagcgacgaggccggccctccctccgcttccaaagaaacgccccccatcgccactatatacatacccccccctctcctcccatccccccaaccct

SEQ ID NO: 44 - DNA, 1026 bp, Artificial Sequence
  hpt (Hygromycin phosphotransferase gene)
atgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccggccgtctggaccgatggctgtgtagaagtactcgccgatagtggaaaccgacgccccagcactcgtccgggatcctctag

SEQ ID NO: 45 - DNA, 977 bp, Artificial Sequence
  delta En/Spm
caacttctgtccaagaccaattgatgccattgggtgtgataggagggcaaatgatgccgtgggcacctcgccagccaggcatttggccaccgatgcaaacacagatgccaccgccgatgccgtggggatttcctcctcgtgggcagtcacaatcaccaggattgccctcacactcaccaggatcagtacgttaagttgatatcctttgcatctctatttgcttcgttgtttaagcagttactagaaaacatgcatgtatatgttgcagtctatgtatatgtttaattagttactcggtaaactaacaaatgtttgtttcttttaaagggttcaggctcacatcatgctagtccgcctccggatcagagcacgtttatggacttattgatgaacacaagtggcggcggctccaatgacccaccaacagaatgaattaatatggaggcttgtgtggaacttactatgattgcgttttgtatggactttaacttgttttagatggatttgaacttctttcgtatggacttgaacttgtatgaatattgaatatggtgcttgtgttatgttatgttgaatatggtgcttgtgttgtgatatattgaatgttgtgcttatattgtgctgttatggaggcttcccatccggggagggagaaaaataaaattggatattaaaaaaaattattcactaagagtgtcggcccccacactcttatatgcgcccaggtagcttactgatgtgcgcgcagtaagagtgacggccacggtactggccgacacttttaacataagagtgtcggttgcttgttgaaccgacacttttaacataagagcgtcggtccccacacttctatacgaataagagcgtccattttagagtgacggctaagagtgtcggtcaaccgacactcttatacttagagtgtcggcttatttcagtaagagtgtggggttttggccgaagctgggcccgctagc

SEQ ID NO: 46 - DNA, 769 bp, Oryza sativa
ccagtgaaagcagtgaattgaagcattcccgaaacccactggaatgatctagtactcactctacgatgtacagtgaagtaatacttcaaaactggtgtaatttggtatgccaaaaggactccatagtttcacgacatatttccaaacggttcaggatcagtactgcccatctgcctggggcccacactagcgggcaattggttctcgtagtttctcgttctcaatcaatcattccatactcgctatcccctccatcacagaataaatgcaacaatgagtttccgtgtacaaatttaatcgttcgtcttatttaaaatattttttaaaaaactaaaaaacaaaagtcacgcataaagtactattcatgttttataatctaataacagtataaatactaatcataaaaaaaaattcaaataagatggacgattaaagttgaacactgaaattcatggctgcttttgttttgagactgagggagtacacgataagatttgatcgcaatcaaagtaacctacatcaaagaagcaagatatgtgggggaaaaatgaatactctagagcaaattaaggtgagccccgctttgtagaggctgatggagtactggagcgacggaagcgaagcagatcgagtgtgctgtaaagcgaaacgagcaagaaccagagaagtccagagatttcaggacagattagttgtgaacctataaatatcctgcctcattccccaacctccatccatcgagccaagactgaagcatttgatcgagctc

SEQ ID NO: 47 - DNA, 2216 bp, Artificial Sequence
  AcTPase4x gene with intron and SV40 NLS
atggctccaaagaagaagagaaaggtcatggccatcgtgcatgagccacagccacaaccgcaaccacagccagagcctcagcctcaacctcagcctgagccagaggaggaggccccacaaaagcgcgctaagaagtgcacctccgacgtctggcagcacttcaccaagaaggagatcgaggtcgaggttgacggcaagaagtacgtccaggtctggggccactgcaacttcccaaactgcaaggccaagtacagggccgagggccatcatggcacatccggctttaggaaccacctcaggacctcccactctctcgtgaagggccaactctgcctcaagtccgagaaggaccacggcaaggacatcaacctcatcgagccgtacaagtacgacgaggtggtgtccctcaagaagctccacctcgccatcattatgcacgagtacccgttcaacatcgtcgagcacgcctacttcgtcgagttcgtgaagtccctcaggccgcacttcccgatcaagtctagagtgaccgcccgcaagtacatcatggacctctaccttgaggagaaggagaagctctacggcaagctcaaggacgtgcagtccaggttctccaccacgatggacatgtggaccagctgccagaacaagtcctacatgtgcgtgaccatccactggatcgacgacgactggtgcctccagaagaggatcgtcggcttcttccatgtggccggcagacatacaggccagaggctctcccaaaccttcaccgcgatcatggtcaagtggaacatcgagaagaagctgttcgccctctccctcgataacgcctccgctaatgaggtggccgtgcacgacatcatcgaggacctccaggacaccgactccaaccttgtgtgcgacggcgccttctttcatgttcgctgcgcctgccacatcctcaacctggttgctaaggatggcctcgccgtgatcgccggcaccattgagaagatcaaggccatcgtcctcgccgtcaagtcctctccacttcagtgggaggagctgatgaagtgcgcgtccgagtgcgatctcgacaagtccaagggcatcagctacgccgtgtccaccaggtggaatagcacctacctcatgctccgcgacgccctctactacaagccagccctcatcaggctcaagacctccgatccaagacggtatgtttgtctcaattgttgtacatgtcatcattataaattctcaattaatcaaatgtcaattattgtagctacgatgccatctgcccaaaggccgaggagtggaagatggcgctcaccctcttcaagtgcctgaagaagttcttcgacctcaccgagctgctctccggcacccaatactctaccgccaacctcttctacaagggcttctgcgagatcaaggacctgatcgcccaatggtgcgtgcacgagaagttcgtgattcgcagaatggccgtggccatgagcgagaagtttgagaagtactggaaggtgtccaatatcgccctcgcggtggcctgcttcctcgatccaaggtacaagaagatcctgatcgagttctacatgaagaagtttcacggcgactcctacaaggtgcacgtcgacgatttcgtgcgcgtgatccgcaagctctaccagttctactcctcctgctctccatccgccccaaagaccaagaccaccaccaacgacagcatggacgacaccctcatggagaacgaggacgacgagttccagaactacctccacgagctgaaggactacgaccaggtcgagagcaacgagctggacaagtacatgtccgagccgctcttgaagcactccggccagttcgatatcctctcatggtggcgcggcagagtggccgagtacccaatcctcacacagatcgccagagatgtgctcgccatccaggtgtcaacagtggcttctgagtctgccttttccgctggcggcagggtggtggatccatacagaaatagactcggctccgagatcgtcgaggccctcatctgcactaaggactgggtggcagcctctaggaagggcgccacatacttcccgaccatgattggcgaccttgaggtgctcgacagcgttatcgccgctgccaccaaccacgagaaccacatggacgaggatgaggacgccatcgagttcagcaagaacaatgaggacgtggccagcggctcctccccatga

SEQ ID NO: 48 - DNA, 253 bp, Artificial Sequence
  tNOS (NOS terminator)
gatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 49 - DNA, 712 bp, Artificial Sequence
  EGFP gene
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgt

SEQ ID NO: 50 - DNA, 2998 bp, Oryza sativa
gctccctcctcacttactctgtcgtggctccactcgctctaccctaactgactcggtcttgcaggagccggaggtgcgctcgggccgtggcgaccgccgccgccgccgctggccacggggccgctcatcagaggagcgggatttggtcgatcaggtgggggggggggttgattttgtgacgaaccatttgtagctctgcatcgtctggttgggggagggttgggtggggatgcgagggagctgatgatgctgtggttgttgtgtgtttcgtagggatgatttggtggtgccgaggtcgccctacttccctgtggagtatgcgtcggggcaggaacgcgggccatcgcccatggtgatggagcggttccagagcgtcgtcagccagctcttccagcacgtacgggagcactagattgttgcacaagtttatttgtggagggttctgtgtgctgtctgactctgtgtgattgtggaacagaggattatccggtgtggtggacccgtggaggatgatatggcgaacatcatcgttgcccagctgctatatctcgatgccatcgatcctaacaaggtgaaaatgatgaataagacgctatccgacatccaattcctatttcctataccaatagctgccaaagctttgttacgaactgcttactctaaaaatactgaggcgatgaagggagtggtgctcactgtatttacaattttacatgaactatatggtgtcttgttttaatggatgattgttgttaatccccagataaaatggtccttttatctgtcaatactcaataccagacactaaagtaccattgctactcactttgggttatatatgagtccttgttagtcctagcttaggctgtgattcagttatgtagttgtgacctcaaaccttatggtgaggcttgtattgcgatttcatctaccaggacctgattccatggcaccatatattcatgatttcgtacttacattcagtactcgattcttgacttcctctcaacattcctgtaggatatcattatgtatgtgaattctcctggaggatcagtgacagctggtaataactcatccattttttttcatagctccatctcactcttggaacatttgtttcacaccaaactgcattttgtgtttagggatggccatattcgacacgatgaagcatatcagacctgatgtttccacagtttgtattggacttgctgcaaggtatttagtactgaaaaactgatatccttaattggcaattgatttggatttgtgacgctctgcaatttatgcatcttataatgtttaattctttagcattctgttcaattattcaataagggtttcagttgctatattctggtcaacatgtaaacatgcacctgtttccagttgtatatcccgcctaattccttctcctttcataatgtgggaagcacaccaaatcattgattgtatcaggcatgttctattcagtataatcagcctcaatctaggattttgggttgactgagatcaaattttgtgctgcgcatgttagcaaagaagctttgtaccctcatacctccttaatcaaaatataatttttcaaactttatgtttgtgagaatccatatggatgttgataacaggagagtttcttcatttcatgtactttatcaatgtttagccaacaatgcaaattcaggagtgcttatcagtattccaggcacacagtttattagtaaaacatgataactgttcagctgcaaatctgtaaccatttctttctcattgtacttttggaatgctctcttttagctatccatcttgccttaagttgcattgatttcggttatgcactcattttcactgttttgtttccctttttggcagtatgggagcttttctgcttagtgctgggacaaaaggtaattgagtgattgttaatgcaagtactacatttattctacaactagactgatctgaatccttgaatactgtacttgtgaatgtaacacagggaagcgatacagcttacctaactcaagaataatgatccatcaacctctcggaggagcccaaggacaagagactgatcttgagatccaggtaaatccacaaagttaaattgtgtttttatcaagatcaggacgacatctcaatagcaaataattaaccatatcattgtttctctcttgccggtatatggttcgatctacatgaaatacatttatttccacaagcgaatagcaactccgctggctttctattgttacttgtatcaccagaagatttttctgtcactgattttccattatttcttttaggctaatgagatgctgcatcacaaggctaacctgaatggatacctagcataccacactgggcagcccctagataagatcaacgtagatactgaccgtgattacttcatgagcgcgaaggaggcaaaggagtatggtctaattgatggagttatcatgaatccccttaaagcccttcaaccgcttcctgcttctagttagccatggagtgctcaatctccacggagcattttttggttatcttttagaactgttattgcatccactgtttttattagcttggcaagatagttttgcgattccacaagcaaccacatcctgaggcttcaaagtttgtacaatacagatgtactactaggaggatatcttctgcgatgaatattgcaacttatttgatgtactattaggaggatatcttctgcgatgaatattgcaacttatttgatactccctctgtttcatgccaagtcgatttgattttttttccttagacaattttctgtaggtttgactaaatttatcaaaaaattagcataatctacaacactaaattagttctattaaaataacattgaatatattttgataatatgtttgttgtattaaaatgttgctatattttttatatacttgatcaaattttaaaaaagattatctataaaaaaattaaaacgatttataatatgatgaaa

SEQ ID NO: 51 - DNA, 35 bp, Artificial Sequence
  PCR Primer
gctctccgcgttttgggtacaggcaattcagacag

SEQ ID NO: 52 - DNA, 30 bp, Artificial Sequence
  PCR Primer
acctgatcgaccaaatcccgctcctctgat

SEQ ID NO: 53 - DNA, 50 bp, Artificial Sequence
  PCR Primer
agtcatttaaatcgtcgacgtgactgtttagtgaaaataactcatgaaat

SEQ ID NO: 54 - DNA, 52 bp, Artificial Sequence
  PCR Primer
cggaagaggggagtgcagctgaggagcggcagagatctcggggcggggttcg

SEQ ID NO: 55 - DNA, 60 bp, Artificial Sequence
  PCR Primer
gccgctcctcagctgcactcccctcttccgcagggatgaaagtaggatgggaaaatcccg

SEQ ID NO: 56 - DNA, 42 bp, Artificial Sequence
  PCR Primer
tgacgcggccgcttcttacatgggctgggcctcagtggttat

SEQ ID NO: 57 - DNA, 50 bp, Artificial Sequence
  PCR Primer
agtcatttaaatcgtcgacgtattactgttattcctgcatgcagtggaag

SEQ ID NO: 58 - DNA, 30 bp, Artificial Sequence
  PCR Primer
ccctcgcccaaaacccccacgagccgcggc

SEQ ID NO: 59 - DNA, 30 bp, Artificial Sequence
  PCR Primer
gctatactaccccattcggtgtctgagcgg

SEQ ID NO: 60 - DNA, 60 bp, Artificial Sequence
  PCR Primer
accgaccgttaccgaccgttttcatccctagctccctcctcactctactctgtcgtggct

SEQ ID NO: 61 - DNA, 49 bp, Artificial Sequence
  PCR Primer
tgacggcgcgcctttcatcatattataaatcgttttaatttttttatag

SEQ ID NO: 62 - DNA, 42 bp, Artificial Sequence
  PCR Primer
agtccctcgaggtgctccagatttatatggattttatctatg

SEQ ID NO: 63 - DNA, 60 bp, Artificial Sequence
  PCR Primer
agccacgacagagtagagtgaggagggagctagggatgaaaacggtcggtaacggtcggt

SEQ ID NO: 64 - DNA, 42 bp, Artificial Sequence
  PCR Primer
tgacggcgcgcctgcagctacgcactactgttaatcaaagtt

SEQ ID NO: 65 - DNA, 30 bp, Artificial Sequence
  PCR Primer
aggtgctttacatcttgcaatgtaagtacc

SEQ ID NO: 66 - DNA, 30 bp, Artificial Sequence
  PCR Primer
gtccagttggctggactggggtgggttggg

SEQ ID NO: 67 - DNA, 30 bp, Artificial Sequence
  PCR Primer
caccaaccacgagaaccacatggacgagga

SEQ ID NO: 68 - DNA, 30 bp, Artificial Sequence
  PCR Primer
ttctcccacagtcccactaatgtgaaatga

SEQ ID NO: 69 - DNA, 30 bp, Artificial Sequence
  PCR Primer
catgctagtccgcctccggatcagagcacg

SEQ ID NO: 70 - DNA, 30 bp, Artificial Sequence
  PCR Primer
gagccatggccaaggttctgcaggagctcg

SEQ ID NO: 71 - DNA, 40 bp, Artificial Sequence
  PCR Primer
agtcaagctttgtgtctggtggacgtccgtggtgcgcgcg

SEQ ID NO: 72 - DNA, 45 bp, Artificial Sequence
  PCR Primer
atccgaattcgctgggcaacgatgatgttcgccatatcatcctcc

SEQ ID NO: 73 - DNA, 30 bp, Artificial Sequence
  PCR Primer
ggacaaaagtggggattaatgatcagcgag

SEQ ID NO: 74 - DNA, 30 bp, Artificial Sequence
  PCR Primer
gtagatggtgggttgcatgagtcaccaatt

SEQ ID NO: 75 - DNA, 30 bp, Artificial Sequence
  PCR Primer
ttgtcaatggccatgactctacgccagaca

SEQ ID NO: 76 - DNA, 30 bp, Artificial Sequence
  PCR Primer
ctgtgggggacatgttcgtcgacctccggt

SEQ ID NO: 77 - DNA, 30 bp, Artificial Sequence
  PCR Primer
cttgacacttttgtattgagtttcgctgta

SEQ ID NO: 78 - DNA, 30 bp, Artificial Sequence
  PCR Primer
tgtgtctggtggacgtccgtggtgcgcgcg

SEQ ID NO: 79 - DNA, 30 bp, Artificial Sequence
  PCR Primer
ttgggtggggatgcgagggagctgatgatg

SEQ ID NO: 80 - DNA, 30 bp, Artificial Sequence
  PCR Primer
cttgttttaatggatgattgttgttaatcc

SEQ ID NO: 81 - DNA, 30 bp, Artificial Sequence
  PCR Primer
ccagttgtatatcccgcctaattccttctc

SEQ ID NO: 82 - DNA, 30 bp, Artificial Sequence
  PCR Primer
tgaatgtaacacagggaagcgatacagctt

SEQ ID NO: 83 - DNA, 30 bp, Artificial Sequence
  PCR Primer
cccttcaaccgcttcctgcttctagttagc

SEQ ID NO: 84 - DNA, 30 bp, Artificial Sequence
  PCR Primer
gcaacttatttgatactccctctgtttcat

SEQ ID NO: 85 - DNA, 30 bp, Artificial Sequence
  PCR Primer
aaatttgatcaagtatataaaaaatatagc

SEQ ID NO: 86 - DNA, 30 bp, Artificial Sequence
  PCR Primer
ttagccttgtgatgcagcatctcattagcc

SEQ ID NO: 87 - DNA, 30 bp, Artificial Sequence
  PCR Primer
ctggaatactgataagcactcctgaatttg

SEQ ID NO: 88 - DNA, 30 bp, Artificial Sequence
  PCR Primer
cactgatcctccaggagaattcacatacat

SEQ ID NO: 89 - DNA, 30 bp, Artificial Sequence
  PCR Primer
agatatagcagctgggcaacgatgatgttc

SEQ ID NO: 90 - DNA, 30 bp, Artificial Sequence
  PCR Primer
ccgcgttcctgccccgacgcatactccaca

SEQ ID NO: 91 - DNA, 30 bp, Artificial Sequence
  PCR Primer
gagaatagtgtgtgtcttcttcgagactcg

SEQ ID NO: 92 - DNA, 30 bp, Artificial Sequence
  PCR Primer
ccctaaattttaagcgtcactttgaatcgc

SEQ ID NO: 93 - DNA, 30 bp, Artificial Sequence
  PCR Primer
ctacatcctagcactactaaacatggttag

SEQ ID NO: 94 - DNA, 30 bp, Artificial Sequence
  PCR Primer
ccataatatatttgtagccgaatccaccac

SEQ ID NO: 95 - DNA, 30 bp, Artificial Sequence
  PCR Primer
caataattttcataatggtaaggtacagtt

SEQ ID NO: 96 - DNA, 30 bp, Artificial Sequence
  PCR Primer
tgctgtcaacagcatgtgacctcaataaga

SEQ ID NO: 97 - DNA, 30 bp, Artificial Sequence
  PCR Primer
gagcagccgcatcagtagtcatcagtgtgc

SEQ ID NO: 98 - DNA, 30 bp, Artificial Sequence
  PCR Primer
tcccttacttgttgtaagtagggctagacg

SEQ ID NO: 99 - DNA, 50 bp, Artificial Sequence
  PCR Primer
agtcatttaaatcgtcgacgctccgccaagcccgccgccgacctctccgg

SEQ ID NO: 100 - DNA, 60 bp, Artificial Sequence
  PCR Primer
agtctctttcaggggcatctcctgcgtctttgcgcgcgtcaaggttcccggcatacgtcc

SEQ ID NO: 101 - DNA, 76 bp, Artificial Sequence
  PCR Primer
gcaggagatgcccctgaaagagactgaaaggcagtgaagaaagttgcagggatgaaagtaggatgggaaaatcccg

SEQ ID NO: 102 - DNA, 50 bp, Artificial Sequence
  PCR Primer
agtcatttaaatcgtcgacgatcagtgtgcttgctctctctcccatcgct

SEQ ID NO: 103 - DNA, 30 bp, Artificial Sequence
  PCR Primer
ctttctgctcctccactccagttacaggca

SEQ ID NO: 104 - DNA, 30 bp, Artificial Sequence
  PCR Primer
ggtgggacagccgttgaggtagggccgaga

SEQ ID NO: 105 - DNA, 60 bp, Artificial Sequence
  PCR Primer
ccgaccgttaccgaccgttttcatccctaatcagatctttagacgtgtctcagcaaggtc

SEQ ID NO: 106 - DNA, 42 bp, Artificial Sequence
  PCR Primer
tgacggcgcgccgggagatgaagcttcggaggatccctatgc

SEQ ID NO: 107 - DNA, 60 bp, Artificial Sequence
  PCR Primer
accttgctgagacacgtctaaagatctgattagggatgaaaacggtcggtaacggtcggt

SEQ ID NO: 108 - DNA, 47 bp, Artificial Sequence
  PCR Primer
tgacggcgcgccgccataatttaatatgtgtgtgagacttctctttc

SEQ ID NO: 109 - DNA, 30 bp, Artificial Sequence
  PCR Primer
ccgccgcgactagctagctagctcggaggt

SEQ ID NO: 110 - DNA, 30 bp, Artificial Sequence
  PCR Primer
tgctagaaacatggatgtctagagtgcatg

SEQ ID NO: 111 - DNA, 40 bp, Artificial Sequence
  PCR Primer
agtcaagcttgacgttgggcagtccatactagagagttac

SEQ ID NO: 112 - DNA, 45 bp, Artificial Sequence
  PCR Primer
atccgaattctattggtgatggaaattatagtcaaggaggatcac

SEQ ID NO: 113 - DNA, 30 bp, Artificial Sequence
  PCR Primer
tttccagaggtggacatgatgaaggagcgc

SEQ ID NO: 114 - DNA, 30 bp, Artificial Sequence
  PCR Primer
aggcggccatggcgattaacagcgatgtgc

SEQ ID NO: 115 - DNA, 30 bp, Artificial Sequence
  PCR Primer
cattcttaccaattatcagtggaagctgac

SEQ ID NO: 116 - DNA, 30 bp, Artificial Sequence
  PCR Primer
gaggctgccatccatgtgtggaggttgaag

SEQ ID NO: 117 - DNA, 30 bp, Artificial Sequence
  PCR Primer
gacgttgggcagtccatactagagagttac

SEQ ID NO: 118 - DNA, 30 bp, Artificial Sequence
  PCR Primer
agccaagggtcgtctagccctacttacaac

SEQ ID NO: 119 - DNA, 30 bp, Artificial Sequence
  PCR Primer
cctaatggacagatctgacgctcttcagtg

SEQ ID NO: 120 - DNA, 30 bp, Artificial Sequence
  PCR Primer
cgaaggccgccgggccgcagctgaagctgc

SEQ ID NO: 121 - DNA, 30 bp, Artificial Sequence
  PCR Primer
gcatcgagcccatgtccaccatctccgccg

SEQ ID NO: 122 - DNA, 30 bp, Artificial Sequence
  PCR Primer
ctgctgaaggcgtctctggagtcgacgacg

SEQ ID NO: 123 - DNA, 30 bp, Artificial Sequence
  PCR Primer
tgtaacccggtcgaagcttcacccaatgca

SEQ ID NO: 124 - DNA, 30 bp, Artificial Sequence
  PCR Primer
ccctggatcaagtagtctggtacacacgaa

SEQ ID NO: 125 - DNA, 30 bp, Artificial Sequence
  PCR Primer
atatctctctgagttccacacatagtaacc

SEQ ID NO: 126 - DNA, 30 bp, Artificial Sequence
  PCR Primer
tgcacccacacacagctagagcactgacac

SEQ ID NO: 127 - DNA, 30 bp, Artificial Sequence
  PCR Primer
atcaacatattgtcaccatctcagttcatc

SEQ ID NO: 128 - DNA, 30 bp, Artificial Sequence
  PCR Primer
aaaatccactccctccggatagagaagggt

SEQ ID NO: 129 - DNA, 30 bp, Artificial Sequence
  PCR Primer
aggaagaaggcgtcgtcgagggcgcgcgcg

SEQ ID NO: 130 - DNA, 30 bp, Artificial Sequence
  PCR Primer
gctaagcctaagctaatagcagaggcaacc

SEQ ID NO: 131 - DNA, 30 bp, Artificial Sequence
  PCR Primer
gattgaagatggaaacagagtcagcagcgg

SEQ ID NO: 132 - DNA, 30 bp, Artificial Sequence
  PCR Primer
caacaacccatcagctctttgggacaaaac

SEQ ID NO: 133 - DNA, 30 bp, Artificial Sequence
  PCR Primer
ggttactatgtgtggaactcagagagatat

SEQ ID NO: 134 - DNA, 30 bp, Artificial Sequence
  PCR Primer
gtcagcttccactgataattggtaagaatg

SEQ ID NO: 135 - DNA, 30 bp, Artificial Sequence
  PCR Primer
ctttggcagtgactccaagtacacttccgg

SEQ ID NO: 136 - DNA, 30 bp, Artificial Sequence
  PCR Primer
ccagcctccacagctcgccgaacacagtgg

SEQ ID NO: 137 - DNA, 30 bp, Artificial Sequence
  PCR Primer
tgcctgtaactggagtggaggagcagaaag

SEQ ID NO: 138 - DNA, 40 bp, Artificial Sequence
  PCR Primer
agtcaagcttccggaaacctcctcggattccattgcccag

SEQ ID NO: 139 - DNA, 39 bp, Artificial Sequence
  PCR Primer
tgacgaattcgacgccattgaggtcgaagacgccacggg

SEQ ID NO: 140 - DNA, 30 bp, Artificial Sequence
  PCR Primer
atggtagatctgagggtaaatttctagttt

SEQ ID NO: 141 - DNA, 30 bp, Artificial Sequence
  PCR Primer
tcacacgtgatggtgatggtgatggctagc

SEQ ID NO: 142 - DNA, 30 bp, Artificial Sequence
  PCR Primer
gatatgtcctgcgggtaaatagctgcgccg

SEQ ID NO: 143 - DNA, 30 bp, Artificial Sequence
  PCR Primer
ggtacccctggattttggttttaggaatta

SEQ ID NO: 144 - DNA, 30 bp, Artificial Sequence
  PCR Primer
cgtgctctgatccggaggcggactagcatg

SEQ ID NO: 145 - DNA, 26 bp, Oryza sativa
tgccgctcctcaggttcactcccctc

SEQ ID NO: 146 - DNA, 26 bp, Oryza sativa
tgccgctcctcagctgcactcccctc

SEQ ID NO: 147 - DNA, 50 bp, Oryza sativa
ctctgccgctcctcaggttcactcccctcttccccctccctcctcactct

SEQ ID NO: 148 - DNA, 50 bp, Oryza sativa
ctctgccgctcctcagctgcactcccctcttcccgcctccctcctcactc

SEQ ID NO: 149 - DNA, 48 bp, Oryza sativa
ctctgccgctcctcagctgcactcccctcttccctccctcctcactct

SEQ ID NO: 150 - DNA, 44 bp, Oryza sativa
ctctgccgctcctcagctgcactcccctcttccctcctcactct

SEQ ID NO: 151 - DNA, 27 bp, Oryza sativa
  CDS (1)..(27)
gggaaccttagcgcaaggaaagacgca

SEQ ID NO: 152 - protein, 9 aa, Oryza sativa
GNLSARKDA

SEQ ID NO: 153 - DNA, 27 bp, Oryza sativa
  CDS (1)..(27)
gggaaccttgacgcgcgcaaagacgca

SEQ ID NO: 154 - protein, 9 aa, Oryza sativa
GNLDARKDA

SEQ ID NO: 155 - DNA, 3000 bp, Artificial Sequence
  OsRacGEF1 5′ side homologous region for HR
ctccgccaagcccgccgccgacctctccggtttgttcttcctaccaaacaaactctcctctctctgctgtttccttgttgcgtagatctcactccacccctcacgagatgcttcggtttggggcttttgctttccagaggtggacatgatgaaggagcgcttcgccaagctgctgctcggcgaggacatgtccgggagcggcaagggcgtctgcaccgcgctcgccatctccaacgccatcaccaacctctccggtacggtcatgaacccccacattgccgtgatttgcgacgaggagagtggtgaataatgttgctgctgctgttgttgttgtttccgaatccgcagccactgtgttcggcgagctgtggaggctggagccgatggcttcggcgaggaaggcgatgtggacgagggagatggactggctgctctccgtggccgactccatcgtcgagctcaccccgtcaatccaggagctccccgacggcgggggacagttcgaggtcatggtgccgcgcccccgcagcgacctctacatgaacctcccggcgctcaagaagctcgatgcgatgctcctcgccatgattgacgggttcaaggagaccgagttctggtatgtggatagggggatcgtggttgatgacagcggcgggccgttctcgtcgtcgtcatcctcctgcgggcggccgtcggtgcggcaggaggagaaatggtggctgccatgcccgcgggtgccgcccaaggggttgtcagaggatgcgaggaggaagctgcaacaggacagggattgtgcgaaccagatactgaaggcggccatggcgattaacagcgatgtgctcgcggagatggagatcccggaagtgtacttggagtcactgccaaaggtaaacaatcatttcagattatctactcattggtactactagttactaataactggtgctctatactagaaaaaacaccactggtgctggtaaaaatgggccaatgtgttaaattgcagtgaacaggacttcttctcaccatacatgcttgtaattgtagctcaactggtgaaagtgggagtgcctttatggcaatgtggtccgttcgcttgagctagctgtgttgctatcacactatcatctgtcttaaatccaatttatttccattcttgtttgtacagcctgccatttttttcacaaggaatcaattcatgtgtagtagagttaaagtcactgtttttccttgttacagaaataagctaattaatgtctttccagtgcaattctcatgtattttgtgagggggtattagcatattgcacactgtcttggtttacttcttgctgccttatatggcaagatgattttagcatctgatctggaatcttgcaaccagtattccattttaatgaaaagttgaaaacattcttaccaattatcagtggaagctgacagttgatggcatttagaatgcaaagaacttcatcatataaattagcttatgatggagagtattgtaaacattaaacagatacctgggcgaagctcaaattcggcttctttcttgtctgtgcttgtacaccatcacgcgcaaaaataagctagtgatgcatagtattctagttaaggcattaatttcccaaatgtaattcatgtataggttgctcaacctggtcatgaatatggcatctaggcgttgacttttaggcttgttgtgtaatgtgctttagaagacgcaatattgaaattccatggcatctttttaacttttgttctgtaattgaatataaattatttgaactgtatctttaaatgaatattcaagtcgcttctgatatcctttactgtgtgtttatgtgcttaatgagtaccttcctaaaagagttgttcttaactggtaatgatatctctctgagttccacacatagtaacctcttcttttttttttctgcagagtggtaaatcttgcttgggtgagataatttaccgatatataacagctgaacaattctcaccggagtgcctcctcgattgcttggatctgtcatcagaacaccacacacttgaagtagctaacagaatagaggctgccatccatgtgtggaggttgaagggccaaaagaaatccacgcctcaagcaaaatcaaagaaatcttggggtggaaaagtgaagggacttgttggagatacagaaaagagtcatgttttgtcccaaagagctgatgggttgttgcaaagcttaagattgcgataccctggtttgccacaaacctctctcgacatgaacaagatccagtataacaaggttattcctcacattttatcttagttgaatatgtcaaacaagttgggcacttgggcttagagttagcattgtatggttcattcaccttctatttcatcaaaaccgagcatgctagcatgccatactaagatctgaattatatgaatagttacattagaacctttctgtttttaatatcacttaacttattctcttcaaaaaaaatcagtttactgtcaacataatcacaatattcatctttttttcttgtgtttcttgtccaggacgttgggcagtccatactagagagttactcaagggttctggagagcttggcatttaacataattgccagaattgatgatgtaatttatgtagatgatgcaacaaagaagtctgccgctgctgactctgtttccatcttcaatcgtggcattggcgtaccagttcagaagaggatctccccaagcccgttctcaattcagcatacaccatatgcctctccatttgctacacccactttctgctcctccactccagttacaggcagccctggaagagtacagcctccattgaacaaagacaacttaccgacaaaacaagaggttaaagttgagaagctgttttctggtgatatcgagaaggtctggacgtatgccgggaaccttgacgcgcgcaaagacgcaggagatgcccctgaaagagactgaaaggcagtgaagaaagttg

SEQ ID NO: 156 - DNA, 3900 bp, Artificial Sequence
  OsRacGEF1 3′ side homologous region for HR
atcagatctttagacgtgtctcagcaaggtcaacaggtttcaacacatcctgttctggtctctagctagattttccctgtacagaagcaacatgattcatttttttgtgctttcttagcgagccaagggtcgtctagccctacttacaacaagtaagggaatgactctttgagtcttgatcgcatctgaacaagtcatagtttcacgtagctgcctctgtcggacatgtactcttcttgttcatgtttatactagctggtttagttagcagcaagtgattgaacttcagatgctgcagttctttttttttttttttgtccctggaggcatcggttccctaacaatcaactgcacttgcagtgcaatgtatcatgattttctgatgctgttgtatatggcacctaatggacagatctgacgctcttcagtgactctgaagactttcttatatcaactgtgaatctgtgatcgatcacaactgctgaaggcttcacagttttgttggtgatcctccttgactataatttccatcaccaatattttcttctctgcagctgcaacttgcaagcaggttgcctctgctattagcttaggcttagctccatggaagaagcaaagaataccttgttccagaatcacacggcgacattgccagattgccaactaaaccctcaggtgaaagtaacttcatgtgcaaatataccatgcctaatttgatcatcaagtccatggcaacatcttgcttgattacttctccaatcttgcaatcaaatggctactacaatataagctgtgcattgcaatgctgttcgagacgtgtgctttaactgttcgacagtgacctgagaactgagaagagaagactagagttgtagccacacgaccagaccggcatggccgccgccgccgcggcgaacgacacgacgccggcgaaggccgccgggccgcagctgaagctgctggtggacaagcggtcgcggcgcgtgctgtacgcggaggcgcgcaaggacgcggtggacttcctcatcggcctcctccgcgtgcccgcggggctcgccgcgcgcgtcctcgccagccacggcgtcgccgcgccgggctccctcgccacgctctacgccgccgcgcgcgccctcgacgacgccttcttcctcgcgtcgccgccctccccgggcccagaccgccgcgacgcggtgctcgcccccgccgttctcccctccgccgcgcttcccctgctcggcgagcgccccccgccgccgccgccgccgccgccgaagcggtactaccggtgcaacgcgtacgcgatgccgtgccggtcgaacccgctgaacgtgacggacacggcgggcctcgcgtgcccggggtgccggcagccgatgacggtggagatgaagtgggcggccggcggcggcagcaagccggcggaggaggaggaggcggcggccggcggcgagggcgggtacgtgaaggaggtggtgacgtacctggtgatggacgacctgagcatcgagcccatgtccaccatctccgccgtgatgctgctcaagaagttcgacgtcaaggattgctccgccctcgacgagatgaccgtcgacctgggccccaaagaggtaaacttttgcaacccttctctatccggagggagtggattttgatcgttattcgcatttcactcaaataattatgaaataaaattaataagatatattaatatgtgatacgtcactccacaaatatataagttcaaattcaacttctacatatcgtataaaaataaaacaaatttgactgtgaatatacattaactagctgtagtttaactcaataagattaattatggtatgctcatttggctatggttcgtacatattagacaagttagtgcatcatgtttaatatgtttttatagtaggttgtacccccatgttaagttcttctatcactgatataatttattcatttatttattactcattgtgtaccatgatcatgtgtgttgtgcagtgtgtgaagctgctgaaggcgtctctggagtcgacgacggcattgacggatgtgttcagcggcggcgtctccattgataggcttgagtgagcaggcgaggtcgcggatgcacacgtgtgcgagctggctgatgaactgagatggtgacaatatgttgatatgcaatggagaatccaattcatgttttcaccatgaaaccatggtacatgtatgcaaatgttattctttgcacaaaaagtaacttaatgatcacaaagaaaacaggggttaaagttcaaaagatcttctctctctcaacaaaaaatattgcaaaagatctctctcgctaccaatgattgaagggtcgtgtggacccccgtttttataacaagaatcaatcgtgtaatcagtaccattttcctggatcaagtggtctggtacacacgaacaacaaaagagcaacaaaaggcgatcgatccaatgctgccattagccaatactctacacatatgtaacccggtcgaagcttcacccaatgcaatattattaatcatatagttgtgttattatattttgagatacgaataaatatactgtattgtgtcagtgctctagctgtgtgtgggtgcatgcatgatctgatgatatgcttatttcccgtggaaaagtctgcttatatttgtatataattaaggaaccatgcaaatgacgtatataattgcatcaaggtccacaatagacagtatataccaatggtcactagatcgattcctgattcttgagtagtcaacaagaaaaatacaaagttgtgttcttacggggaaagaatatggcaggtcagtagtcgcctaatctcccggtctctagctagcacacaacacgttctctactactcaatcaacacaggttgatgatgggttagtgtggaccccgtttttataacaggaatcaatcgtgtaaacagtaccatttccctggatcaagtagtctggtacacacgaactcatagctgaaatatcaagtgcacatacacgagagtctagtacgaattattacatcataagcccgcaggcacagtcttaaatcatcggaccgagcggtccggaatacattccaaaaacagaagtaggtaaggcagcggagttaaggcagcggtatgccattgccacaggcaacgaccgtactaagacctactgagcgccatcgtcttcatctccctcctggtagtaggcatagggatcctccgaagcttcatctcccgcttctgattatatttgcaagggtgagtaccaaccgtactcagcaagccaccacagcaatgatgcacatgacagaggaattcaaaggatggctatggttcttttgcgcaaagcaagttttgtaattcttttcacaagcctaagacctagcatagactgatcaagttttaataccagtgttcatattaaacaatgatggttctgtccaccatccattgtgatcccaaggatagcttcccgccattgagtcgttatggttttctgaggacgtccatattcccgcctttcaggaagtggctccatcagcataaaaatcatcatgcaatatcccatcccacacaagttaaagaatttagagtctagccaagtgtaatacatgtcccggtgctcaataaccgcgagcacggctattcgaatagatttggtttactcacactgcagtggatgtacactttacccgcactccgcgactgcccaacacatgagcctcgtcccaacacatgagacgcgccacggtaaagccttttgataacctcgcattggcagtacccgctccatgaacttaaatcctcatgcactctagacatccatgtttctagcagtgagaggagttctggcgctcccgggaaagagaagtctcacacacatattaaattatggc

SEQ ID NO: 157 - DNA, 30 bp, Artificial Sequence
  PCR Primer
accaccctctcccggataagaggcgcaacc

SEQ ID NO: 158 - DNA, 30 bp, Artificial Sequence
  PCR Primer
tgtggaatcgcaaaactatcttgccaagct

SEQ ID NO: 159 - DNA, 83 bp, Oryza sativa
gccgggaaccttgacgcgcgcaaagacgcaggagatgcccctgaaagagactgaaaggcagtgaagaaagttcttcagatctt

SEQ ID NO: 160 - DNA, 83 bp, Oryza sativa
aagatctgaagaactttcttcactgcctttcagtctctttcaggggcatctcctgcgtctttgcgcgcgtcaaggttcccggc

SEQ ID NO: 161 - DNA, 80 bp, Oryza sativa
gccgggaaccttgacgcgcgcaaagacgcaggagatgcccctgaaagagactgaaaggcagtgaagaaagttcagatctt

SEQ ID NO: 162 - DNA, 454 bp, Oryza sativa
atggcgaccaccaccaccaccccctcctcctctctcaccgcccctctcctccgcccgagctcgaacgcgaaccccgccccgagatctctgccgctcctcaggagccggaggtgcgctcgggccgtggcgaccgccgccgccgccgctggccacggggccgctcatcagaggagcgggatttggtcgatcagggatgatttggtggtgccgaggtcgccctacttccctgtggagtatgcgtcggggcaggaacgcgggccatcgcccatggtgatggagcggttccagagcgtcgtcagccagctcttccagcacaggattatccggtgtggtggacccgtggaggatgatatggcgaacatcatcgttgcccagctgctatatctcgatgccatcgatcctaacaaggatatcattatgtatgtgaattctcctggaggatcagtgacagctg

SEQ ID NO: 163 - DNA, 537 bp, Oryza sativa
atggcgaccaccaccaccaccccctcctcctctctcaccgcccctctcctccgcccgagctcgaacgcgaaccccgccccgagatctctgccgctcctcagctgcactcccctcttccccctccctcctcactctactctgtcgtggctccactcgctctaccctaactgactcggtcttgcaggagccggaggtgcgctcgggccgtggcgaccgccgccgccgccgctggccacggggccgctcatcagaggagcgggatttggtcgatcagggatgatttggtggtgccgaggtcgccctacttccctgtggagtatgcgtcggggcaggaacgcgggccatcgcccatggtgatggagcggttccagagcgtcgtcagccagctcttccagcacaggattatccggtgtggtggacccgtggaggatgatatggcgaacatcatcgttgcccagctgctatatctcgatgccatcgatcctaacaaggatatcattatgtatgtgaattctcctggaggatcagtgacagctg

SEQ ID NO: 164 - DNA, 34 bp, Oryza sativa
cctcaggttcactcccctcttccccctccctcct

SEQ ID NO: 165 - DNA, 32 bp, Oryza sativa
cctaactgactcggtcttgcaggagccggagg

SEQ ID NO: 166 - DNA, 10 bp, Oryza sativa
gagccggagg

SEQ ID NO: 167 - DNA, 31 bp, Oryza sativa
cctcagctgcactcccctcttcctccctcct

SEQ ID NO: 168 - DNA, 32 bp, Oryza sativa; identical to SEQ ID NO: 165